(19) United States
(12) Patent Application Publication
(10) Pub. No.: US 2026/0206762 A1
(43) Pub. Date: Jul. 23, 2026

(54) PYRIMIDINONE DERIVATIVES AS PESTICIDAL COMPOUNDS

(71) Applicant: BASF SE, Ludwigshafen am Rhein (DE)

(72) Inventors: Rizwan Shabbir Shaikh, Navi Mumbai (IN); Pulakesh Maity, Navi Mumbai (IN); Ashokkumar Adisechan, Navi Mumbai (IN)

(21) Appl. No.: 19/137,325

(22) PCT Filed: Dec. 4, 2023
(86) PCT No.: PCT/EP2023/084039

§ 371 Date: Jun. 10, 2025

(30) Foreign Application Priority Data
Dec. 14, 2022 (EP) 22213537.8
Dec. 14, 2022 (EP) 22213538.6

(51) Int. Cl. A01N 43/90; A01P 17/00; C07D 487/04

(52) U.S. Cl. CPC A01N 43/90; A01P 17/00; C07D 487/04

(57) ABSTRACT

The invention relates to a compound of formula (I) G(I), wherein the variables are defined in the specification. It also relates to a pesticidal mixture comprising the compound of formula (I); the use of compounds of formula (I) as an agrochemical pesticide; a method for combating or controlling invertebrate pests, a method for protecting growing plants from attack or infestation by invertebrate pests, seed comprising a compound of the formula (I); the use of a compound of the formula (I) for protecting growing plants from attack or infestation by invertebrate pests; and a method for treating or protecting an animal from infestation or infection by invertebrate pests.

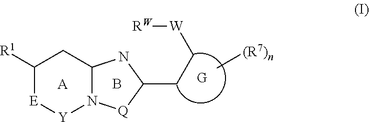

The invention relates to compounds of formula (I) or an agrochemically or verterinarily acceptable salt, stereoisomer, tautomer, or N-oxide thereof

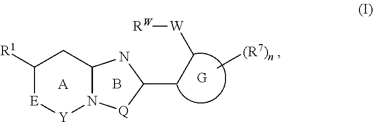

wherein the variables are as defined below. The invention also relates to the use of compounds of formula (I) as an agrochemical pesticide; to pesticidal compositions comprising a compound of formula (I) and another pesticidal ingredient; to a method for combating or controlling invertebrate pests, which method comprises contacting said pest or its food supply, habitat or breeding grounds with a pesticidally effective amount of at least one compound of the formula (I) the pesticidal mixture; to a method for protecting growing plants from attack or infestation by invertebrate pests, which method comprises contacting a plant, or soil or water in which the plant is growing, with a pesticidally effective amount of at least one compound of the formula (I) or the pesticidal mixture; and to seeds comprising a compound of the formula (I) or the pesticidal composition in an amount of from 0.1 g to 10 kg per 100 kg of seeds; to a use of a compound of the formula (I) or of the pesticidal compositions, for protecting growing plants from attack or infestation by invertebrate pests; and to a method for treating or protecting an animal from infestation or infection by invertebrate pests which comprises bringing the animal in contact with a pesticidally effective amount of a compound of the formula (I).

Invertebrate pests and in particular insects, arachnids and nematodes destroy growing and harvested crops and attack wooden dwelling and commercial structures, thereby causing large economic loss to the food supply and to property. Accordingly, there is an ongoing need for new agents for combating invertebrate pests.

WO2017/167832 and WO2018/206479 disclose bicyclic pyrimidone compounds and their pesticidal activity. The invention differs from theses disclosures in that residue R¹ is a halogen. WO2021/204577 and WO2022128524 disclose bicyclic pyrimidone compounds and their pesticidal activity, wherein residue R¹ is among others selected from halogen, and wherein R⁷ is among others selected from “—C(CN)R⁷R⁸ and C₃-C₆-cycloalkyl, which is substituted with CN and which either does not have any further substituents, or which is further substituted with one or more, same or different substituents R⁹”. WO'77 does not disclose the specific combination of R¹ being halogen and R⁷ as presently claimed.

Due to the ability of target pests to develop resistance to pesticidally-active agents, there is an ongoing need to identify further compounds, which are suitable for combating invertebrate pests such as insects, arachnids and nematodes. Furthermore, there is a need for new compounds having a high pesticidal activity and showing a broad activity spectrum against a large number of different invertebrate pests, especially against difficult to control insects, arachnids and nematodes. Furthermore, there is a need for compounds that are environmentally friendly and display a favorable toxicological profile.

It is therefore an object of the invention to identify and provide compounds, which exhibit a high pesticidal activity, have a broad activity spectrum against invertebrate pests, and show environmentally and toxicologically advantageous characteristics.

It has been found that these objects can be achieved by substituted bicyclic compounds of formula (I), as depicted and defined below, including their stereoisomers, their salts, in particular their agriculturally or veterinarily acceptable salts, their tautomers and their N-oxides.

In a first aspect, the invention relates to a compound of formula (I),

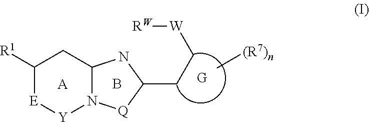

wherein
the rings A and B are fully unsaturated.
Y is C═X, wherein X is O;
E is N(R³);
Q is N, N(R⁵) or CH;
G is phenyl or pyridyl;
W is S, S(O), S(O)₂, S(O)(NR^{W}), S(O)(NH);
R¹ is halogen;
R³, R⁵ are independently C₁-C₆-alkyl, C₁-C₆-alkoxy, C₂-C₆-alkenyl, C₂-C₆-alkynyl, C₃-C₆-cycloalkyl, C₃-C₆-cycloalkyl-C₁-C₄-alkyl, which are unsubstituted or halogenated;
each R⁷ is independently H, halogen, OH, CN;
   C₁-C₆-alkyl, C₃-C₆-cycloalkyl, C₂-C₆-alkenyl, C₂-C₆-alkynyl, which groups are unsubstituted, or halogenated;
   a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more R^{H};
   phenyl, which is unsubstituted, or substituted with one or more R^{J};
   OR^{K};
   —C(CN)(CH₃)₂, or 1-cyanocyclopropyl.
each R^{H} is independently halogen, CN; C₁-C₃-alkyl, C₁-C₃-haloalkyl;
each R^{J} is independently halogen, CN; C₁-C₆-alkyl, C₁-C₆-alkoxy, which groups are unsubstituted, or halogenated; or in case of two adjacent substituents R^{J} which are C₁-C₆-alkoxy, the substituents form an alkyl bridge between the two oxygens, which is unsubstituted or halogenated;
each R^{K} is C₁-C₆-alkyl, C₂-C₆-alkenyl, C₂-C₆-alkynyl, C₃-C₆-cycloalkyl, which groups are unsubstituted or substituted with one or more, same or different substituents selected from halogen, CN;
each R^{W} is independently C₁-C₄-alkyl, or cyclopropyl;
the index n is 1, 2, or 3, or 4, if G is phenyl, or 1, 2, or 3 if G is pyridyl;
and the N-oxides, stereoisomers, tautomers and agriculturally or veterinarily acceptable salts thereof.

In another aspect, the invention relates to a compound of formula (I),

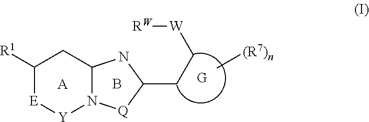

wherein
the rings A and B are fully unsaturated.
Y is C═X, wherein X is O;
E is N(R³);
Q is N, N(R⁵) or CH;
G is phenyl or pyridyl;
W is S, S(O), S(O)₂, S(O)(NR^{W});
R¹ is halogen;
R³, R⁵ are independently C₁-C₆-alkyl, C₁-C₆-alkoxy, C₂-C₆-alkenyl, C₂-C₆-alkynyl, C₃-C₆-cycloalkyl, C₃-C₆-cycloalkyl-C₁-C₄-alkyl, which are unsubstituted or halogenated;
each R⁷ is independently H, halogen, OH, CN;
   C₁-C₆-alkyl, C₃-C₆-cycloalkyl, C₂-C₆-alkenyl, C₂-C₆-alkynyl, which groups are unsubstituted, or halogenated;
   a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more R^{H};
   phenyl, which is unsubstituted, or substituted with one or more R^{J};
   OR^{K};
   —C(CN)(CH₃)₂, or 1-cyanocyclopropyl.
each R^{H} is independently halogen, CN; C₁-C₃-alkyl, C₁-C₃-haloalkyl;
each R^{J} is independently halogen, CN; C₁-C₆-alkyl, C₁-C₆-alkoxy, which groups are unsubstituted, or halogenated;
each R^{K} is C₁-C₆-alkyl, C₂-C₆-alkenyl, C₂-C₆-alkynyl, C₃-C₆-cycloalkyl, which groups are unsubstituted or substituted with one or more, same or different substituents selected from halogen, CN;
each R^{W} is independently C₁-C₄-alkyl, cycloalkyl, or cyclopropyl;
the index n is 1, 2, or 3, or 4, if G is phenyl, or 1, 2, or 3 if G is pyridyl;
and the N-oxides, stereoisomers, tautomers and agriculturally or veterinarily acceptable salts thereof.

The compounds of the formula (I), and their agriculturally acceptable salts are highly active against animal pest, i.e. harmful arthropodes and nematodes, especially against insects and acaridae which are difficult to control by other means.

Moreover, the invention relates to and includes the following embodiments:
compositions comprising at least one compound of formula (I) as defined above and a liquid or solid carrier;
agricultural and veterinary compositions comprising an amount of at least one compound of formula (I) or an enantiomer, diasteromer or salt thereof as defined above;
methods for combating invertebrate pests, infestation, or infection by invertebrate pests, which method comprises contacting said pest or its food supply, habitat or breeding grounds with a pesticidally effective amount of at least one compound of formula (I) as defined above or a composition thereof;
methods for controlling invertebrate pests, infestation, or infection by invertebrate pests, which method comprises contacting said pest or its food supply, habitat or breeding grounds with a pesticidally effective amount of at least one compound of formula (I) as defined above or a composition comprising at least one compound of formula (I);
methods for preventing or protecting against invertebrate pests comprising contacting the invertebrate pests, or their food supply, habitat or breeding grounds with substituted imidazolium compounds of the general formula (I) as defined above or a composition comprising at least one compound of formula (I) as defined above or a composition comprising at least one compound of formula (I);
methods for protecting crops, plants, plant propagation material and/or growing plants from attack or infestation by invertebrate pests comprising contacting or treating the crops, plants, plant propagation material and growing plants, or soil, material, surface, space, area or water in which the crops, plants, plant propagation material is stored or the plant is growing, with a pesticidally effective amount of at least one compound of formula (I) as defined above or a composition comprising at least one compound of formula (I);
non-therapeutic methods for treating animals infested or infected by parasites or preventing animals of getting infected or infested by parasites or protecting animals against infestation or infection by parasites which comprises orally, topically or parenterally administering or applying to the animals a parasiticidally effective amount of a compound of formula (I) as defined above or a composition comprising at least one compound of formula (I);
methods for treating, controlling, preventing or protecting animals against infestation or infection by parasites by administering or applying orally, topically or parenterally to the animals a compound of the general formula (I) as defined above or a composition comprising at least one compound of formula (I);
seed comprising a compound of formula (I) as defined above, in an amount of from 0.1 g to 10 kg per 100 kg of seed;
the use of the compounds of formula (I) as defined above for protecting growing plants or plant propagation material from attack or infestation by invertebrate pests;
the use of compounds of formula (I) or the enantiomers, diastereomers or veterinary acceptable salts thereof for combating parasites in and on animals;
a process for the preparation of a veterinary composition for treating, controlling, preventing or protecting animals against infestation or infection by parasites which comprises adding a parasiticidally effective amount of a compound of formula (I) or the enantiomers, diastereomers and/or veterinary acceptable salt thereof to a carrier composition suitable for veterinary use;
the use of a compound of formula (I) or the enantiomers, diastereomers and/or veterinary acceptable salt thereof for the preparation of a medicament for treating, controlling, preventing or protecting animals against infestation or infection by parasites.

All the compounds of formula (I) and, if applicable, their stereoisomers, their tautomers, their salts or their N-oxides as well as compositions thereof are particularly useful for controlling invertebrate pests, in particular for controlling arthropods and nematodes and especially insects. Therefore, the invention relates to the use of a compound of formula (I) as an agrochemical pesticide, preferably for combating or controlling invertebrate pests, in particular invertebrate pests of the group of insects, arachnids or nematodes.

The term “compound(s) according to the invention” or “compound(s) of formula (I)” as used in the invention refers to and comprises the compound(s) as defined herein and/or stereoisomer(s), salt(s), tautomer(s) or N-oxide(s) thereof. The term “compound(s) of the invention” is to be understood as equivalent to the term “compound(s) according to the invention”, therefore also comprising stereoisomer(s), salt(s), tautomer(s) or N-oxide(s) of compounds of formula (I). As used herein, the term “compound(s) of the invention” or “compound(s) according to the invention” refers to the compound(s) of formula (I) as defined above, which are also referred to as “compound(s) of formula I” or “compound(s) I” or “formula I compound(s)”, and includes their salt(s), tautomer(s), stereoisomer(s), and N-oxide(s).

The term “composition(s) according to the invention” or “composition(s) of the invention” encompasses composition(s) comprising at least one compound of formula (I) according to the invention as defined above, therefore also including a stereoisomer, an agriculturally or veterinary acceptable salt, tautomer or an N-oxide of the compounds of formula (I).

The compounds of the invention may be amorphous or may exist in one or more different crystalline states (polymorphs) or modifications which may have a different macroscopic properties such as stability or show different biological properties such as activities. The invention includes both amorphous and crystalline compounds of the formula (I), mixtures of different crystalline states or modifications of the respective compound I, as well as amorphous or crystalline salts thereof.

The compounds of the formula (I) may have one or, depending on the substitution pattern, more centers of chirality, in which case they are present as mixtures of enantiomers or diastereomers. The invention provides both the single pure enantiomers or pure diastereomers of the compounds of formula (I), and their mixtures and the use according to the invention of the pure enantiomers or pure diastereomers of the compound of formula (I) or its mixtures. Suitable compounds of the formula (I) also include all possible geometrical stereoisomers (cis/trans isomers) and mixtures thereof. Cis/trans isomers may be present with respect to an alkene, carbon-nitrogen double-bond or amide group. The term “stereoisomer(s)” encompasses both optical isomers, such as enantiomers or diastereomers, the latter existing due to more than one center of chirality in the molecule, as well as geometrical isomers (cis/trans isomers). The invention relates to every possible stereoisomer of the compounds of formula (I), i.e. to single enantiomers or diastereomers, as well as to mixtures thereof.

Depending on the substitution pattern, the compounds of the formula (I) may be present in the form of their tautomers. Hence the invention also relates to the tautomers of the formula (I) and the stereoisomers, salts, tautomers and N-oxides of said tautomers.

Salts of the compounds of the formula (I) are preferably agriculturally and/or veterinary acceptable salts. They can be formed in a customary method, e.g. by reacting the compound with an acid of the anion in question if the compound of formula (I) has a basic functionality or by reacting an acidic compound of formula (I) with a suitable base.

Suitable agriculturally or veterinary useful salts are especially the salts of those cations or the acid addition salts of those acids whose cations and anions, respectively, do not have any adverse effect on the action of the compounds according to the invention. Suitable cations are in particular the ions of the alkali metals, preferably lithium, sodium and potassium, of the alkaline earth metals, preferably calcium, magnesium, and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formulae I with an acid of the corresponding anion, preferably of hydrochloric acid, hydrobromic acid, sulfuric acid, phosphoric acid or nitric acid.

The term “N-oxide” includes any compound of the invention which has at least one tertiary nitrogen atom that is oxidized to an N-oxide moiety.

The organic moieties groups mentioned in the above definitions of the variables are—like the term halogen—collective terms for individual listings of the individual group members. The prefix Cₓ-C_{y} indicates in each case the possible number of carbon atoms in the group. “Halogen” will be taken to mean F, Cl, Br, and I, preferably F.

The terms compound(s) of formula (x) and compound(s) (x), wherein x is a roman or arab number are used herein interchangeably and refer to a single or several compounds as defined by the respective formula (x).

The term “substituted with”, e.g. as used in “partially, or fully substituted with” means that one or more, e.g. 1, 2, 3, 4 or 5 or all of the hydrogen atoms of a given radical have been replaced by one or more, same or different substituents as subsequently defined. Accordingly, for substituted cyclic moieties, e.g. 1-cyanocyclopropyl, one or more of the hydrogen atoms of the cyclic moiety may be replaced by one or more, same or different substituents.

The term “halogen” means fluoro, chloro, bromo, iodo, preferably fluoro, chloro, bromo. Other radical groups may be substituted with halogen, i.e. halogenated, which is understood be fully or partly substituted with one or more halogen atoms, unless indicated otherwise.

The term “Cₓ-C_{y}-alkyl” as used herein (and also in Cₓ-C_{y}-alkylamino, di-Cₓ-C_{y}-alkylamino, Cₓ-C_{y}-alkylaminocarbonyl, di-(Cₓ-C_{y}-alkylamino)carbonyl, Cₓ-C_{y}-alkylthio, Cₓ-C_{y}-alkylsulfinyl and Cₓ-C_{y}-alkylsulfonyl) refers to a branched or unbranched saturated hydrocarbon group having n to m, e.g. 1 to 10 carbon atoms, preferably 1 to 6 carbon atoms, e.g. methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-di-methylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. C₁-C₄-alkyl means for example methyl, ethyl, propyl, 1-methylethyl, butyl, 1-methylpropyl, 2-methylpropyl or 1,1-dimethylethyl.

The term “Cₓ-C_{y}-haloalkyl” as used herein (and also in Cₓ-C_{y}-haloalkylsulfinyl and Cₓ-C_{y}-halo-alkylsulfonyl) refers to a straight-chain or branched alkyl group having n to m carbon atoms, e.g. 1 to 10 in particular 1 to 6 carbon atoms (as mentioned above), where some or all of the hydrogen atoms in these groups may be replaced by halogen atoms as mentioned above, e.g. C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like. The term C₁-C₁₀-haloalkyl in particular comprises C₁-C₂-fluoroalkyl, which is synonym with methyl or ethyl, wherein 1, 2, 3, 4 or 5 hydrogen atoms are substituted with fluorine atoms, such as fluoromethyl, difluoromethyl, trifluoromethyl, 1-fluoroethyl, 2-fluoroethyl, 2,2-difluoroethyl, 2,2,2-trifluoroethyl and pentafluoromethyl.

Similarly, “Cₓ-C_{y}-alkoxy” and “Cₓ-C_{y}-alkylthio” (or Cₓ-C_{y}-alkylsulfenyl, respectively) refer to straight-chain or branched alkyl groups having n to m carbon atoms, e.g. 1 to 10, in particular 1 to 6 or 1 to 4 carbon atoms (as mentioned above) bonded through oxygen (or sulfur linkages, respectively) at any bond in the alkyl group. Examples include C₁-C₄-alkoxy such as methoxy, ethoxy, propoxy, isopropoxy, butoxy, sec-butoxy, isobutoxy and tert-butoxy, further C₁-C₄-alkylthio such as methylthio, ethylthio, propylthio, isopropylthio, and n-butylthio.

Accordingly, the terms “Cₓ-C_{y}-haloalkoxy” and “Cₓ-C_{y}-haloalkylthio” (or Cₓ-C_{y}-haloalkylsulfenyl, resp.) refer to straight-chain or branched alkyl groups having n to m carbon atoms, e.g. 1 to 10, in particular 1 to 6 or 1 to 4 carbon atoms (as mentioned above) bonded through oxygen or sulfur linkages, resp., at any bond in the alkyl group, where some or all of the hydrogen atoms in these groups may be replaced by halogen atoms as mentioned above, e.g. C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoro-methoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy and pentafluoroethoxy, further C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like. Similarly, the terms C₁-C₂-fluoroalkoxy and C₁-C₂-fluoroalkylthio refer to C₁-C₂-fluoroalkyl which is bound to the remainder of the molecule via an oxygen atom or a sulfur atom, respectively.

The term “C₂-C_{y}-alkenyl” as used herein intends a branched or unbranched unsaturated hydrocarbon group having 2 to m, e.g. 2 to 10 or 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “C₂-C_{y}-alkynyl” as used herein refers to a branched or unbranched unsaturated hydrocarbon group having 2 to m, e.g. 2 to 10 or 2 to 6 carbon atoms and containing at least one triple bond, such as ethynyl, propynyl, 1-butynyl, 2-butynyl, and the like.

The term “Cₓ-C_{y}-alkoxy-Cₓ-C_{y}-alkyl” as used herein refers to alkyl having n to m carbon atoms, e.g. like specific examples mentioned above, wherein one hydrogen atom of the alkyl radical is replaced by an Cₓ-C_{y}-alkoxy group; wherein the value of x and y of the alkoxy group are independently chosen from that of the alkyl group.

Accordingly, the term “Cₓ-C_{y}-alkoxy-Cₓ-C_{y}-alkoxy” as used herein refers to alkoxy group having n to m carbon atoms, e.g. like methoxy or ethoxy, wherein one hydrogen atom of the alkoxyl radical is replaced by an Cₓ-C_{y}-alkoxy group; wherein the value of x and y of the alkoxy group are independently chosen from that of the alkoxyl group.

The suffix “-carbonyl” in a group or “C(═O)” denotes in each case that the group is bound to the remainder of the molecule via a carbonyl C═O group. This is the case e.g. in alkylcarbonyl, haloalkylcarbonyl, aminocarbonyl, alkylaminocarbonyl, dialkylaminocarbonyl, alkoxycarbonyl, haloalkoxycarbonyl, alkylcarbonylamino, and hydroxycarbonyl. For example, a hydroxycarbonyl group would refer to a carbonic acid group —C(═O)OH, and an aminocyrbonyl group would refer to an amide group —C(═O)NH₂, both of which are bound to the remainder of the molecule via the carbonyl “C═O” group.

The term “aryl” as used herein refers to a mono-, bi- or tricyclic aromatic hydrocarbon radical such as phenyl or naphthyl, in particular phenyl (also referred as to C₆H₅ as substituent).

The term “C₃-C_{y}-cycloalkyl” as used herein refers to a monocyclic ring of 3- to y-membered saturated cycloaliphatic radicals, e.g. cyclopropyl (cC₃H₅), cyclobutyl (cC₄H,), cyclopentyl (cC₅Hg), cyclohexyl (cC₆H₁₁), cycloheptyl, cyclooctyl and cyclodecyl.

Accordingly, the term “C₃-C_{y}-cycloalkoxy” as used herein refers to a “C₃-C_{y}”-cycloalkyl moiety which is bonded to the rest of the molecule via an oxygen atom, such as in cyclopropoxy, cyclobutoxy, cyclopentoxy and cyclohexoxy.

The term “C₃-C_{y}-cycloalkoxy-Cₓ-C_{y}-alkyl” refers to a Cₓ-C_{y}-alkyl moiety, wherein one hydrogen atom is substituted by a C₃-C_{y}-alkoxy group and wherein the indices x and y are independently chosen according to the indicated meaning.

The term “cycloalkylalkyl” denotes as well as the term “alkyl which may be substituted with cycloalkyl” an alkyl group which is substituted with a cycloalkyl ring, wherein alkyl and cycloakyl are as herein defined.

The term “C₃-C_{y}-cycloalkenyl” as used herein refers to a monocyclic ring of 3- to y-membered partially unsaturated cycloaliphatic radicals.

The term “cycloalkylcycloalkyl” denotes as well as the term “cycloalkyl which may be substituted with cycloalkyl” a cycloalkyl substitution on another cycloalkyl ring, wherein each cycloalkyl ring independently has from 3 to 7 carbon atom ring members and the cycloalkyls are linked through one single bond or have one common carbon atom. Examples of cycloalkylcycloalkyl include cyclopropylcyclopropyl (e.g. 1,1′-bicyclopropyl-2-yl), cyclohexylcyclohexyl wherein the two rings are linked through one single common carbon atom (e.g. 1,1′-bicyclo-hexyl-2-yl), cyclohexylcyclopentyl wherein the two rings are linked through one single bond (e.g. 4-cyclopentylcyclohexyl) and their different stereoisomers such as (1R,2S)-1, 1′-bicyclopropyl-2-yl and (1R,2R)-1,1′-bicyclopropyl-2-yl. The term “carbocycle” or “carbocyclyl” includes, unless otherwise indicated, in general a 3- to 12-membered, preferably a 3- to 8-membered or a 5- to 8-membered, more preferably a 5- or 6-membered mono-cyclic, ring comprising 3 to 12, preferably 3 to 8 or 5 to 8, more preferably 5 or 6 carbon atoms.

The carbocyclic radicals may be saturated, partially unsaturated, or fully unsaturated. Preferably, the term “carbocycle” covers cycloalkyl and cycloalkenyl groups as defined above, for example cyclopropane, cyclobutane, cyclopentane and cyclohexane rings. When it is referred to “fully unsaturated” carbocycles, this term also includes “aromatic” carbocycles. In certain preferred embodiments, a fully unsaturated carbocycle is an aromatic carbocycle as defined below, preferably a 6-membered aromatic carbocycle.

The term “hetaryl” or “aromatic heterocycle” or “aromatic heterocyclic ring” includes mono-cyclic 5- or 6-membered heteroaromatic radicals comprising as ring members 1, 2, 3, or 4 heteroatoms selected from N, O, and S. Examples of 5- or 6-membered heteroaromatic radicals include pyridyl, i.e. 2-, 3-, or 4-pyridyl, pyrimidinyl, i.e. 2-, 4- or 5-pyrimidinyl, pyrazinyl, pyridazinyl, i.e. 3- or 4-pyridazinyl, thienyl, i.e. 2- or 3-thienyl, furyl, i.e. 2- or 3-furyl, pyrrolyl, i.e. 2- or 3-pyrrolyl, oxazolyl, i.e. 2-, 3- or 5-oxazolyl, isoxazolyl, i.e. 3-, 4- or 5-isoxazolyl, thiazolyl, i.e. 2-, 3- or 5-thiazolyl, isothiazolyl, i.e. 3-, 4- or 5-isothiazolyl, pyrazolyl, i.e. 1-, 3-, 4- or 5-pyrazolyl, i.e. 1-, 2-, 4- or 5-imidazolyl, oxadiazolyl, e.g. 2- or 5-[1,3,4]oxadiazolyl, 4- or 5-(1,2,3-oxadiazol)yl, 3- or 5-(1,2,4-oxadiazol)yl, 2- or 5-(1,3,4-thiadiazol)yl, thiadiazolyl, e.g. 2- or 5-(1,3,4-thiadiazol)yl, 4- or 5-(1,2,3-thiadiazol)yl, 3- or 5-(1,2,4-thiadiazol)yl, triazolyl, e.g. 1H-, 2H- or 3H-1,2,3-triazol-4-yl, 2H-triazol-3-yl, 1H-, 2H-, or 4H-1,2,4-triazolyl and tetrazolyl, i.e. 1H- or 2H-tetrazolyl.

The terms “heterocycle”, “heterocyclyl” or “heterocyclic ring” includes, unless otherwise indicated, in general 3- to 12-membered, preferably 3- to 8-membered, 3- to 7-membered, or 5- to 8-membered, more preferably 5- or 6-membered, in particular 6-membered monocyclic heterocyclic radicals. The heterocyclic radicals may be saturated, partially unsaturated, or fully unsaturated. As used in this context, the term “fully unsaturated” also includes “aromatic”. In a preferred embodiment, a fully unsaturated heterocycle is thus an aromatic heterocycle, preferably a 5- or 6-membered aromatic heterocycle comprising one or more, e.g. 1, 2, 3, or 4, preferably 1, 2, or 3 heteroatoms selected from N, O and S as ring members. Examples of aromatic heterocycles are provided above in connection with the definition of “hetaryl”. Unless otherwise indicated, “hetaryls” are thus covered by the term “heterocycles”. The heterocyclic non-aromatic radicals usually comprise 1, 2, 3, 4 or 5, preferably 1, 2 or 3 heteroatoms selected from N, O, and S as ring members, where S-atoms as ring members may be present as S, SO or SO₂, and N-atoms may be oxidized, or non-oxidized. Examples of 5- or 6-membered heterocyclic radicals comprise saturated or unsaturated, non-aromatic heterocyclic rings, such as oxiranyl, oxetanyl, thietanyl, thietanyl-S-oxid (S-oxothietanyl), thietanyl-S-dioxid (S-dioxothiethanyl), pyrrolidinyl, pyrrolinyl, pyrazolinyl, tetrahydrofuranyl, dihydrofuranyl, 1,3-dioxolanyl, thiolanyl, S-oxothiolanyl, S-dioxothiolanyl, dihydrothienyl, S-oxodihydrothienyl, S-dioxodihydrothienyl, oxazolidinyl, oxazolinyl, thiazolinyl, oxathiolanyl, piperidinyl, piperazinyl, pyranyl, dihydropyranyl, tetrahydropyranyl, 1,3- and 1,4-dioxanyl, thiopyranyl, S.oxothiopyranyl, S-dioxothiopyranyl, dihydrothiopyranyl, S-oxodihydrothiopyranyl, S-dioxodihydrothiopyranyl, tetrahydrothiopyranyl, S-oxotetrahydrothiopyranyl, S-dioxotetrahydrothiopyranyl, morpholinyl, thiomorpholinyl, S-oxothiomorpholinyl, S-dioxothiomorpholinyl, thiazinyl and the like. Examples for heterocyclic ring also comprising 1 or 2 carbonyl groups as ring members comprise pyrrolidin-2-onyl, pyrrolidin-2,5-dionyl, imidazolidin-2-onyl, oxazolidin-2-onyl, thiazolidin-2-onyl and the like.

The erms “alkylene”, “alkenylene”, and “alkynylene” refer to alkyl, alkenyl, and alkynyl as defined above, respectively, which are bonded to the remainder of the molecule, via two atoms, preferably via two carbon atoms, of the respective group, so that they represent a linker between two moieties of the molecule. In particular, the term “alkylene” may refer to alkyl chains such as CH₂CH₂, —CH(CH₃)—, CH₂CH₂CH₂, CH(CH₃)CH₂, CH₂CH(CH₃), CH₂CH₂CH₂CH₂, CH₂CH₂CH₂CH₂CH₂, CH₂CH₂CH₂CH₂CH₂CH₂, and CH₂CH₂CH₂CH₂CH₂CH₂CH₂. Similarly, “alkenylene” and “alkynylene” may refer to alkenyl and alkynyl chains, respectively.

The term “5- to 6-membered carbocyclic ring” as used herein refers to cyclopentane and cyclohexane rings.

Examples of 5- or 6-membered saturated heterocyclic rings include: 2-tetrahydrofuranyl, 3-tetrahydrofuranyl, 2-tetrahydrothienyl, 3-tetrahydrothienyl, 2-pyrrolidinyl, 3-pyrrolidinyl, 3-pyrazolidinyl, 4-pyrazolidinyl, 5-pyrazolidinyl, 2-imidazolidinyl, 4-imidazolidinyl, 2-oxazolidinyl, 4-oxazolidinyl, 5-oxazolidinyl, 3-isoxazolidinyl, 4-isoxazolidinyl, 5-isoxazolidinyl, 2-thiazolidinyl, 4-thiazolidinyl, 5-thiazolidinyl, 3-isothiazolidinyl, 4-isothiazolidinyl, 5-isothiazolidinyl, 1,2,4-oxadiazolidin-3-yl, 1,2,4-oxadiazolidin 5 yl, 1,2,4-thiadiazolidin-3-yl, 1,2,4-thiadiazolidin-5-yl, 1,2,4-triazolidin-3-yl,-1,3,4-oxadiazolidin-2-yl, 1,3,4-thiadiazolidin-2-yl, 1,3,4-triazolidin-2-yl, 2-tetrahydropyranyl, 4-tetrahydropyranyl, 1,3-dioxan-5-yl, 1,4-dioxan-2-yl, 2-piperidinyl, 3-piperidinyl, 4-piperidinyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl, 2-morpholinyl, 3-morpholinyl, 2-thiomorpholinyl, 3-thiomorpholinyl, 1-oxothiomorpholin-2-yl, 1-oxothiomorpholin-3-yl, 1,1-dioxothiomorpholin-2-yl, 1,1-dioxothiomorpholin-3-yl.

Examples of 5- or 6-membered partially unsaturated heterocyclyl or heterocyclic rings include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin 3 yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3 dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl.

Examples of 5- or 6-membered fully unsaturated heterocyclic (hetaryl) or heteroaromatic rings are: 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

A “C₂-C_{y}-alkylene” is divalent branched or preferably unbranched saturated aliphatic chain having 2 to m, e.g. 2 to 7 carbon atoms, for example CH₂CH₂, —CH(CH₃)—, CH₂CH₂CH₂, CH(CH₃)CH₂, CH₂CH(CH₃), CH₂CH₂CH₂CH₂, CH₂CH₂CH₂CH₂CH₂, CH₂CH₂CH₂CH₂CH₂CH₂, and CH₂CH₂CH₂CH₂CH₂CH₂CH₂.

The term “alkylamino” as used herein refers to a straight-chain or branched saturated alkyl group having 1 to 10 carbon atoms, preferably 1 to 4 carbon atoms, more preferably 1 to 3 carbon atoms, which is bonded via a nitrogen atom, e.g. an —NH— group.

The term “dialkylamino” as used herein refers to a straight-chain or branched saturated alkyl group having 1 to 10 carbon atoms, preferably 1 to 4 carbon atoms, more preferably 1 to 3 carbon atoms, which is bonded via a nitrogen atom, which is substituted by another straight-chain or branched saturated alkyl group having 1 to 10 carbon atoms, preferably 1 to 4 carbon atoms, more preferably 1 to 3 carbon atoms, e.g. a methylamino or ethylamino group.

The term “alkylthio “(alkylsulfanyl: alkyl-S—)” as used herein refers to a straight-chain or branched saturated alkyl group having 1 to 10 carbon atoms, preferably 1 to 4 carbon atoms (═C₁-C₄-alkylthio), more preferably 1 to 3 carbon atoms, which is attached via a sulfur atom. Examples include methylthio, ethylthio, propylthio, isopropylthio, and n-butylthio.

The term “haloalkylthio” as used herein refers to an alkylthio group as mentioned above wherein the hydrogen atoms are partially or fully substituted by fluorine, chlorine, bromine and/or iodine. Examples include chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like.

The term “alkylsulfinyl” (alkylsulfoxyl: C₁-C₆-alkyl-S(═O)—), as used herein refers to a straight-chain or branched saturated alkyl group (as mentioned above) having 1 to 10 carbon atoms, preferably 1 to 4 carbon atoms (═C₁-C₄-alkylsulfinyl), more preferably 1 to 3 carbon atoms bonded through the sulfur atom of the sulfinyl group at any position in the alkyl group.

The term “(halo)alkylsulfonyl” (alkyl-S(═O)₂—) as used herein refers to a straight-chain or branched saturated (and halogenated) alkyl group having 1 to 10 carbon atoms, preferably 1 to 4 carbon atoms (═C₁-C₄-(halo)alkylsulfonyl), preferably 1 to 3 carbon atoms, which is bonded via the sulfur atom of the sulfonyl group at any position in the (halo)alkyl group.

The term “(halo)alkylsulfanyl” (alkyl-S—) as used herein refers to a straight-chain or branched saturated (and halogenated) alkyl group having 1 to 10 carbon atoms, preferably 1 to 4 carbon atoms (═C₁-C₄-(halo)alkylsulfanyl), preferably 1 to 3 carbon atoms, which is bonded via the sulfur atom of the sulfanyl group at any position in the (halo)alkyl group.

The term “(halo)alkylsulfinyl” (alkyl-S(═O)—) as used herein refers to a straight-chain or branched saturated (and halogenated) alkyl group having 1 to 10 carbon atoms, preferably 1 to 4 carbon atoms (═C₁-C₄-(halo)alkylsulfinyl), preferably 1 to 3 carbon atoms, which is bonded via the sulfur atom of the sulfinyl group at any position in the (halo)alkyl group.

Accordingly, the terms (halo)alkylsulfanylalkyl, (halo)alkylsulfinylalkyl, and (halo)sulfonylalkyl as used for example in “C₁-C₄-alkylsulfanyl-C₁-C₄-alkyl”, “C₁-C₄-alkylsulfinyl-C₁-C₄-alkyl”, and “C₁-C₄-alkylsulfonyl-C₁-C₄-alkyl” relate to (halo)alkylsulfanyl-, (halo)alkylsulfinyl-, or (halo)alkylsulfonyl-groups that are bonded via the sulfur atom of the sulfanyl, the sulfinyl, or the sulfonyl group, respectively, to the alkyl group, which is in turn bonded to the rest of the molecule.

The term “alkylcarbonyl” (C₁-C₆—C(═O)—) refers to a straight-chain or branched alkyl group as defined above, which is bonded via the carbon atom of a carbonyl group (C═0) to the remainder of the molecule.

The term “alkoxycarbonyl” refers to an alkoxygroup group as defined above, which is bonded via the carbon atom of a carbonyl group (C═O) to the remainder of the molecule.

The term “alkylaminocarbonyl” (C₁-C₆—NH—C(═O)—) refers to a straight-chain or branched alkylamino group as defined above, which is bonded via the carbon atom of a carbonyl group (C═O) to the remainder of the molecule. Similarly, the term “dialkylaminocarbonyl” refers to a straight-chain or branched saturated alkyl group as defined above, which is bonded to a nitrogen atom, which is substituted with another straight-chain or branched saturated alkyl group as defined above, which nitrogen atom in turn is bonded via a carbonyl group (C═0) to the remainder of the molecule.

The term “S(O)(NR^{W})” refers to a group

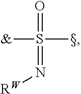

wherein R^{W} is as defined for formula (I) and wherein the symbols “&” and “§” mean the link to the remainder of the molecule. Accordingly, a group R^{W}—W as attached to ring “G” may have the meaning R^{W}—S(O)(NR^{W}), which would refer to a group

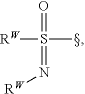

wherein each R^{W} is independently as defined for formula (I) and wherein the symbol “§” means the link to the remainder of the molecule, i.e. ring “G”.

The compounds of formula (I) can be prepared by standard methods of organic chemistry. If certain derivatives cannot be prepared by the processes outlined below, they can be obtained by derivatization of other compounds of formula (I) that are accessible by these methods.

Preparation methods that are generally useful for the preparation of compounds of formula (I) have been disclosed in WO2017/167832, especially p. 4-6 and in the experimental section, WO2021/204577, especially p. 15-26 and in the experimental section, WO2021/099240 and WO2022128524. In the following depicted Processes and Schemes, variables of formulae have a meaning as defined for formula (I) if not described otherwise. The variable “LG” refers to a leaving group, such as Cl, Br, I, triflate, tosylate etc. The variables R⁷¹ and R⁷² each, independently, have a meaning as defined for R⁷ in formula (I)—if not otherwise indicated. The variable A is N or CH.

Compounds of formula (II), corresponding to compounds of formula (I), wherein Q is CH, may be prepared by reaction of compounds of formula (III) with compounds of formula (IV) as displayed under Process 1.

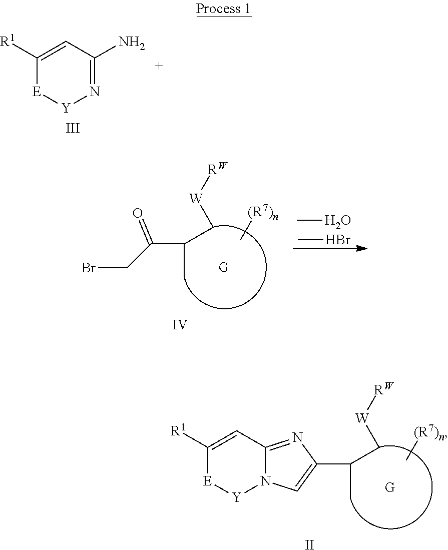

All variables in formulae (Ill), (IV) and (II) have a meaning as defined for formula (I). Reactions of this type have been described in EP3257853A1 and WO 2018206479. The reaction is typically carried out under elevated temperatures of from 50-160° C. in an inert solvent. Suitable solvents are aliphatic hydrocarbons, such as pentane, hexane, cyclohexane, or petrol ether; aromatic hydrocarbons, such as benzene, toluene, o-, m-, and p-xylene; halogenated hydrocarbons, or halogenated aromatic C₆-C₁₀-hydrocarbons, such as CH₂Cl₂, CHCl₃, CCl₄, CH₂ClCH₂Cl, CCl₃CH₃, CHCl₂CH₂C₁, CCl₂CCl₂, or chlorobenzene; ethers, such as CH₃CH₂OCH₂CH₃, (CH₃)₂CHOCH(CH₃)₂, CH₃OC(CH₃)₃ (MTBE), CH₃OCH₃ (DME), CH₃OCH₂CH₂OCH₃, CH₃OC(CH₃)₂CH₂CH₃, dioxane, anisole, 2-methyltetrahydrofuran, tetrahydrofurane (THF), and diethylene glycol; nitriles, such as CH₃CN, and CH₃CH₂CN; alcohols, such as CH₃OH, CH₃CH₂OH, CH₃CH₂CH₂OH, CH₃CH(OH)CH₃, CH₃(CH₂)₃OH, and C(CH₃)₃OH, CH₂(OH)CH₂(OH), CH₃CH(OH)CH₂OH; amides and urea derivatives, such as dimethyl formamide (DMF), N-methyl-2-pyrrolidone (NMP), dimethyl acetamide (DMA), 1,3-dimethyl-2-imidazolidinone (DMI), 1,3-dimethyl-3,4,5,6-tetrahydro-2(1H)-pyrimidinone (DMPU), hexamethylphosphamide (HMPA); moreover dimethyl sulfoxide (DMSO), sulfolane, and water. Mixtures of the above solvents are also possible.

The reaction may be carried out in the presence of a catalyst, such as an acid or a base, preferably a base. Suitable bases are, in general, inorganic bases, such as LiOH, NaOH, KOH, and Ca(OH)₂; alkali metal and alkaline earth metal oxides, such as Li₂O, Na₂O, CaO, and MgO; alkali metal and alkaline earth metal hydrides, such as LiH, NaH, KH and CaH₂; alkali metal and alkaline earth metal carbonates, such as Li₂CO₃, K₂CO₃ and CaCO₃; alkali metal bicarbonates, such as NaHCO₃; organic bases, such as pyrrolidine; tertiary amines, such as diisopropylethylamine, trimethylamine, triethylamine, triisopropylamine and N-methylpiperidine, imidazol, pyridine; substituted pyridines, such as collidine, lutidine and 4-dimethylaminopyridine, and polycyclic amides and amidines, such as 1,8-diazabicycloundec-7-ene (DBU), 1,4-Diazabicyclo[2.2.2]octane (DABCO); alkali metal salts of secondary amines, such as alkali diisopropylamide, alkali bis(trimethylsilyl)amide, alkali tetramethylpiperidene; alcoholates, such as alkali methanolate, alkali ethanolate, alkali isopropanolate, alkali tert-butanolate; alkali metal—alkyl, and alkali metal—aryl salts, such as n-butyl lithium, tert-butyl lithium, phenyl lithium. Mixtures of the aforementioned bases are also possible. The bases are generally employed in catalytic amounts; however, they can also be used in equimolar amounts, in excess or, if appropriate, as solvent. Compounds of formula (III) and compounds of formula (IV) are typically reacted with one another in equimolar amounts. In terms of yield, it may be advantageous to employ an excess of compounds of formula (IV).

Compounds of formula (IVa), falling under the definition of formula (IV), wherein n is 1, W is S(O)₂ and R⁷ is in the para-position, may for example be prepared as depicted under Scheme 1.

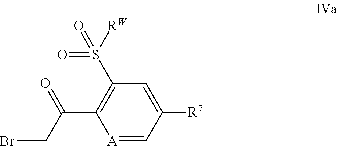

The variable A in Scheme 1 is either N or CH, and R⁷¹ and R⁷², independently, fall under the definition of R⁷.

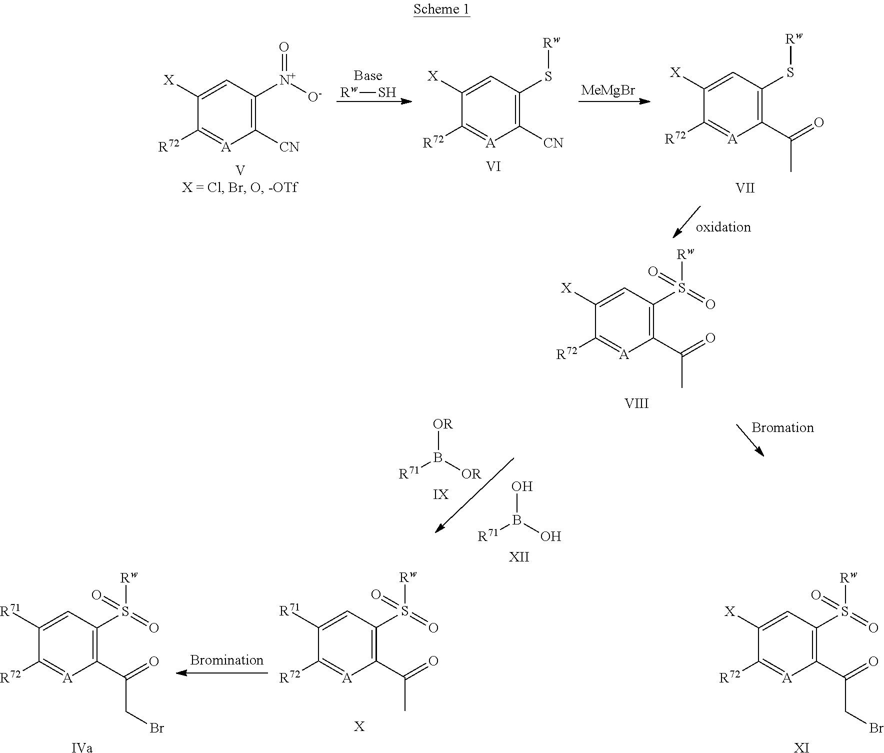

Compound VI can be obtained by the treatment of compound V wherein X is Cl, Br, I, —OTf (triflate), or leaving group by displacement reaction the nitro group (which may also be any other suitable leaving group such as Cl, Br, I, F, —OTf, —OTs (tosylate) etc.) with HS—R^{W} in presence of a base e.g. potassium carbonate, sodium carbonat, cesium carbonate, sodium hydride etc in solvent like DMF, THE and DMSO at cooling to ambient temperature. Such a method has similarly been been described in literature like Tetrahedron Letters, 2014, vol. 55, #22, p. 3295-3298.

Compounds VII may be prepared via conversion of the cyano- to a ketone group by reacting compounds VI with the respective Grignard reagents. To this end, nitrile compounds (VI) may be dissolved in a suitable solvent such as THF, MTBE or toluene at 0° C., followed by the treatment with CH₃MgBr, CH₃MgCl and stirring at 20 to 25° C. as described in WO 2018095795, WO 2016012395 and Tetrahedron Letters 1981, vol. 22, 3815-3818.

Compounds VII can be further oxidised to SO₂ (sulfone), by oxidation reaction, involving reagents such as, m-chloroperoxybenzoic acid, hydrogen peroxide, oxone, sodium periodate, sodium hypochlorite or tert-butyl hypochlorite and the solvents used for the oxidations include aliphatic halogenated hydrocarbons such as dichloromethane and chloroform; alcohols such as methanol and ethanol; acetic acid; water. The amount of the oxidant to be used in the reaction is generally 1 to 3 moles, preferably 1 to 1.2 moles, relative to 1 mole of the sulfide compounds VII to produce the sulfoxide compounds, and preferably 2 to 2.2 moles of oxidant, relative to 1 mole of the sulfide compounds VII as descried in WO 2015/091945 A1, WO 2016107742 and WO 2018095795.

Compounds X, can be prepared by reacting compound VIII in which X is a leaving group such as, for example, chlorine, bromine or iodine (preferably bromine), or an aryl-, alkyl- or haloalkylsulfonate such as trifluoromethanesulfonate, with a reagent R⁷¹—B(OH)₂, R⁷¹—B(OR)₂ where R could be alkyl, aryl, cycloalkyl or pinacole type of cyclic groups of the formula IX.

The reaction may be catalyzed by a palladium based catalyst, involving for example bis(diphenylphosphino)ferrocene]dichloropalladium (II)(1:1) (Pd(dppf)Cl2). DCM complex, tetrakis(triphenylphosphine)palladium(0), bis(dibenzylideneacetone)palladium(O) (Pd(dba)2), tris(dibenzylideneacetone)dipalladium(0) (Pd2(dba)3; optionally in form of its chloroform adduct) or palladium(II) acetate, and a ligand, for example XantPhos ((5-diphenylphosphanyl-9,9-dimethyl-xanthen-4-yl)diphenylphosphane), RuPhos (2-dicyclohexylphosphino2′,6′-diisopropoxybiphenyl), JohnPhos ([1,1-biphenyl]-2-ylbis(1,1-dimethyl-ethyl)phosphine), BINAP (2,2′-bis(diphenylphosphino)-1,1′-binaphthalene), tol-BINAP ([2,2′-bis(di-p-tolyl-phosphino)-1,1′-binaphthyl]) or tri-(o-tolyl)phosphine, in presence of a base, like sodium, potassium or cesium carbonate, or sodium or potassium tert-butylate, in a solvent or a solvent mixture, like, for example dioxane, 1,2-dimethoxyethane or toluene, preferably under inert atmosphere. The reaction temperature can preferentially range from room temperature to the boiling point of the reaction mixture, or the reaction may be performed under microwave irradiation. Such reactions have been described, for example, in Molecules 2012, 17, 4508-4521, Advanced Synthesis & Catalysis (2015), 357(2-3), 361-365, WO2022025242 A1, Org. Lett., 2001, 3, 2757-2759 or Synlett, 2009, 1761-1764.

The reaction temperature can preferentially range from 20 to 25° C. to the boiling point of the reaction mixture, or the reaction may be performed under microwave irradiation. Such reactions have been described, for example, in Advanced Synthesis & Catalysis, 350(3), 391-394; 2008. Compounds Va can be prepared by dissolving compounds X in the possible solvents used are ethyl acetate, chloroform or DCM and the possible reagents used as copper(II) bromide CuBr₂, Br₂, HBr in acetic acid, Trimethyl phenyl ammonium tribromide at ambient temperature or on heating to 60° C. on monitoring to obtain bromo ketone IV. Such procedure can be found in WO 2016107742.

Compounds of formula IX, and XII, are either known compounds, commercially available or can be prepared by known methods, described in the literature, as for example in Green Chemistry (2009), 11(10), 1610-1617, Angewandte Chemie, International Edition (2010), 49(10), 1846-1849, Organic Letters (2011), 13(13), 3312-3315, WO2011149950 A2 etc.

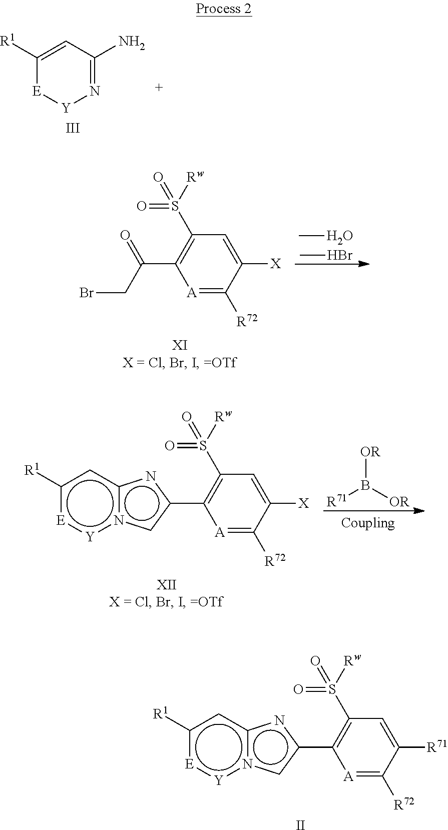

Alternatively, compounds of formula (II), falling under the definition of of formula (I), may be prepared by reaction of compounds of formula (III) with compounds of formula (XI) as displayed under Process 2 to generate formula (XIII) the condensation reaction can be performed as described above to generate compounds (II). Compound (XIII) can be further subjected for the Suzuki, Negishi, or Sonogashira type C—C coupling reaction with R⁷¹—B(OH)2, R⁷¹—B(OR)₂ as described for the synthesis of intermediate (X) above in process 1.

Compounds of formula (XVII), corresponding to compounds of formula (I), wherein Q is NR⁵, can be prepared as displayed under Process 3 below.

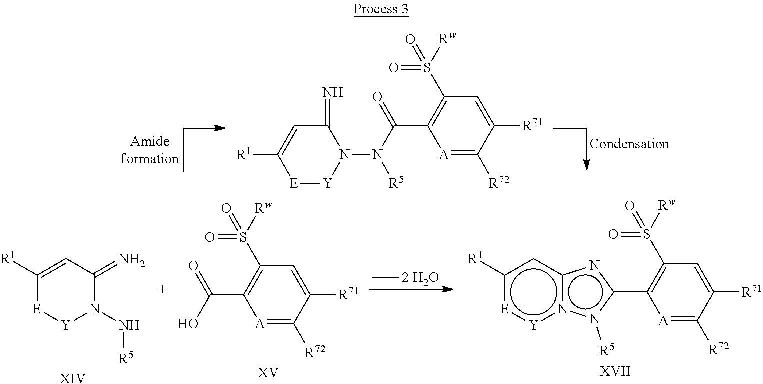

Reactions of this type have been described in EP3257853A1, WO2019/234160 and WO2016162318A1. The reaction is typically carried out at elevated temperatures, e.g. 60 to 160° C., in an inert solvent, optionally in the presence of an acid, or a coupling agent and a base. Suitable solvents are aliphatic hydrocarbons, such as pentane, hexane, cyclohexane, or petrol ether; aromatic hydrocarbons, such as benzene, toluene, o-, m-, and p-xylene; halogenated hydrocarbons, or halogenated aromatic C₆-C₁₀-hydrocarbons, such as CH₂Cl₂, CHCl₃, CCl₄, CH₂ClCH₂Cl, CCl₃CH₃, CHCl₂CH₂Cl, CCl₂CCl₂, or chlorobenzene; ethers, such as CH₃CH₂OCH₂CH₃, (CH₃)₂CHOCH(CH₃)₂, CH₃OC(CH₃)₃ (MTBE), CH₃OCH₃ (DME), CH₃OCH₂CH₂OCH₃, CH₃OC(CH₃)₂CH₂CH₃, dioxane, anisole, 2-methyltetrahydrofuran, tetrahydrofurane (THF), and diethylene glycol; nitriles, such as CH₃CN, and CH₃CH₂CN; alcohols, such as CH₃OH, CH₃CH₂OH, CH₃CH₂CH₂OH, CH₃CH(OH)CH₃, CH₃(CH₂)₃OH, and C(CH₃)₃OH, CH₂(OH)CH₂(OH), and CH₃CH(OH)CH₂OH. Mixtures of the above solvents are also possible.

Suitable acids are in general inorganic acids such as HF, HCl, HBr, H₂SO₄ and HClO₄; Lewis acids, such as BF₃, AlCl₃, FeCl₃, SnCl₄, TiCl₄ and ZnCl₂, moreover organic acids such as HCOOH, CH₃COOH, CH₃CH₂COOH, oxalic acid, toluene sulphonic acid, benzene sulphonic acid, camphor sulphonic acid, citric acid, and CF₃COOH.

Suitable coupling agents are selected from carbodiimides, such as DCC (dicyclohexyl-carbodiimide) and DIC (diisopropylcarbodiimide), benzotriazole derivatives, such as HATU (O-(7-azabenzotriazol-1-yl)-N,N,N′,N′-tetramethyluronium hexafluorophosphate), HBTU ((Obenzotriazol-1-yl)-N,N,N′,N′-tetramethyluronium hexafluorophosphate) and HCTU (1H-benzotriazolium-1-[bis(dimethylamino)methylene]-5-chloro tetrafluoroborate) and phosphonium-derived activators, such as BOP ((benzotriazol-1-yloxy)-tris(dimethylamino) phosphonium hexafluorophosphate), PyBOP ((benzotriazol-1-yloxy)-tripyrrolidinphosphonium hexafluorophosphate) and PyBrOP (bromotripyrrolidinphosphonium hexafluorophosphate).

Suitable solvents are aliphatic hydrocarbons, such as pentane, hexane, cyclohexane, or petrol ether; aromatic hydrocarbons, such as benzene, toluene, o-, m-, and p-xylene; halogenated hydrocarbons, or halogenated aromatic C₆-C₁₀-hydrocarbons, such as CH₂Cl₂, CHCl₃, CC₄, CH₂ClCH₂Cl, CCl₃CH₃, CHCl₂CH₂Cl, CCl₂CCl₂, or chlorobenzene; ethers, such as CH₃CH₂OCH₂CH₃, (CH₃)₂CHOCH(CH₃)₂, CH₃OC(CH₃)₃ (MTBE), CH₃OCH₃ (DME), CH₃OCH₂CH₂OCH₃, CH₃OC(CH₃)₂CH₂CH₃, dioxane, anisole, 2-methyltetrahydrofuran, tetrahydrofurane (THF), and diethylene glycol; nitriles, such as CH₃CN, and CH₃CH₂CN; alcohols, such as CH₃OH, CH₃CH₂OH, CH₃CH₂CH₂OH, CH₃CH(OH)CH₃, CH₃(CH₂)₃OH, and C(CH₃)₃OH, CH₂(OH)CH₂(OH), and CH₃CH(OH)CH₂OH. Mixtures of the above solvents are also possible in presence of suitable bases are those listed for Process 1 above to generate compound of formula XXII. This is followed by condensation described in process 1 to generate compound of formula XVI.

Alternatively, compounds of formula (XV) may be replaced by their corresponding carbonic acid halogenides, e.g. acid chlorides. In this case the reaction is typically carried out in the presence of a base. Suitable bases are those listed for Process 1 above.

Compounds of formula (XIV) and compounds of formula (XV) are typically reacted with one another in equimolar amounts. In terms of yield, it may be advantageous to employ an excess of compounds of formula (XV).

Compounds of formula (XV) may be prepared as described in WO 2019/234160 A1. Compounds of formula (XIV) can be prepared as described in WO2017/167832A1, Example C-4, or Bashandy et al. Journal of Enzyme Inhibition and Medicinal Chemistry, 29(5), 619-627, 2014

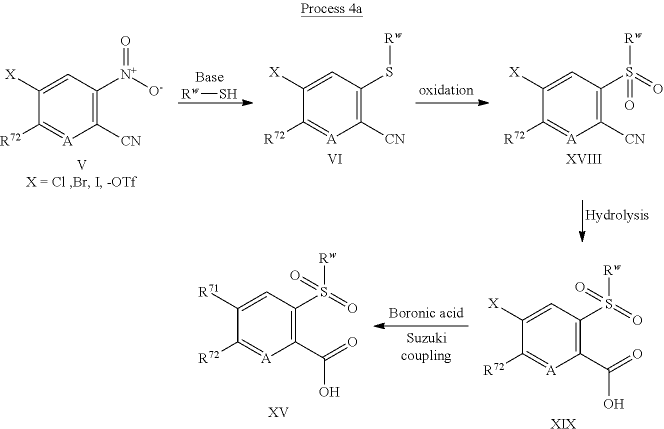

Accordingly, compounds of formula (XV), may for example be prepared as follows. Compounds VI can be oxidised to SO₂ (sulfone), by oxidation reaction, involving reagents such as, m-chloroperoxybenzoic acid, hydrogen peroxide, oxone, sodium periodate, sodium hypochlorite or tert-butyl hypochlorite and the solvents used for the oxidations include aliphatic halogenated hydrocarbons such as dichloromethane and chloroform; alcohols such as methanol and ethanol; acetic acid; water. The amount of the oxidant to be used in the reaction is generally 1 to 3 moles, preferably 1 to 1.2 moles, relative to 1 mole of the sulfide compounds VI to produce the sulfoxide compounds, and preferably 2 to 2.2 moles of oxidant, relative to 1 mole of the sulfide compounds VI as descried in WO 2015/091945 A1, WO 2016107742 and WO 2018095795. Hydrolysis of compounds of formula (XVIII), as defined in formula I, and in which X is a leaving group, in particular those compounds wherein X is a halogen (even more preferably chlorine, bromine or iodine), for example through heating (XVIII) in concentrated acid, such as concentrated hydrochloric acid HCl cone, or sulfuric acid H2SO4 cone., preferably in the presence of water, optionally in the presence of an inert solvent, such as acetic acid or ethers (for example tetrahydrofuran, ethylene glycol dimethyl ether or 1,4-dioxane) gave XIX. Such hydrolysis conditions, and variants thereof, are known to a skilled person. Suzuki coupling (as described for the synthesis of compound II or X) can be used to generate compound XV, which have a meaning as defined for formula (I), was reacted with compound XIV to generate (XVII) by direct condensation reaction or via intermediate XVI by amide formation reaction followed by condensation reaction.

Compounds of formula (XXI), may be prepared by reaction of compounds of formula (XIV) with compounds of formula (XIX) as displayed under Process 5.

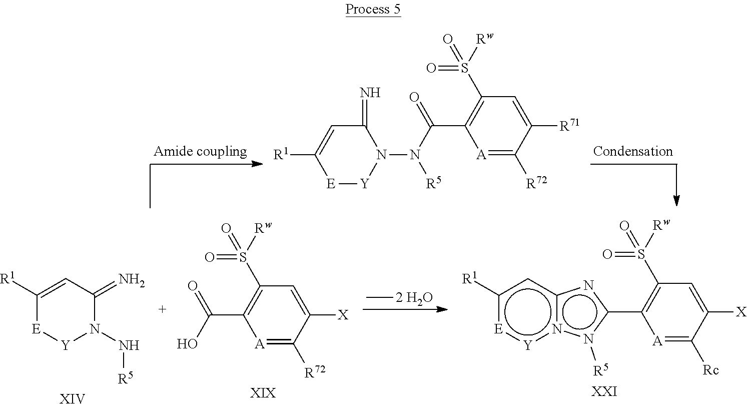

The amide formation reaction to generate compound of formula (XX) followed by condensation to generate compound of formula XXI and/or direct reaction of compound XIV with XIX to generate XXI can be performed as discussed under process 4.

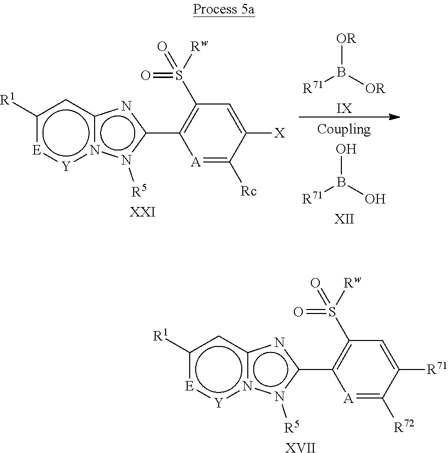

Compounds of formula (XXII), falling under the definition of formula (I), may be prepared by reaction of compounds of formula (XXI) with compounds of formula (IX or XII) under Suzuki type coupling reaction as described under process 1 for the generation compound XXI or II.

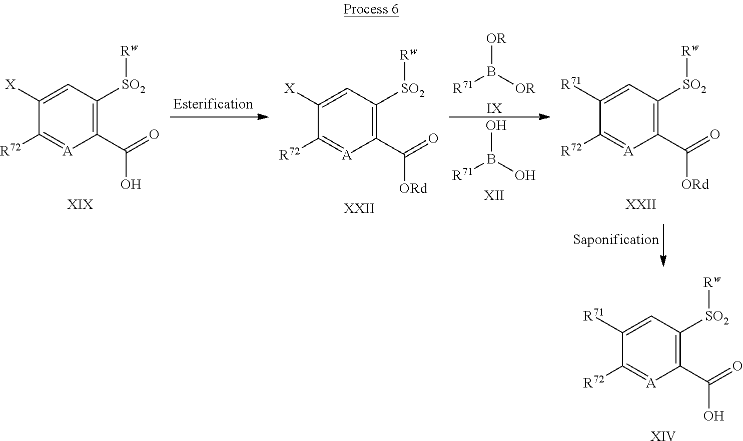

Compounds of formula (XV), may be prepared (process 6) by esterification of compounds of formula (XIX), to generate compounds XXII in which Rd is C₁-C₆-alkyl, under conditions known to a person skilled in the art for example under acidic condition by refluxing in ethanol, methanol or other alcoholic solvents, or under basic condition using potassium carbonate, caesium carbonate, sodium carbonate, sodium hydroxide, potassium hydroxide and by adding electrophiles containing the corresponding alkyl halide Rd-X. Compounds XXII are then converted to compounds XXIII by Suzuki type C—C coupling with boronic acid XII or boronic acid ester IX (as described in process 1) to generate compounds (XXIII). Subsequently, compounds XXIII are converted to compounds XIV by a saponification reaction of compounds XXIII under conditions known to a person skilled in the art (using for example conditions such as: aqueous sodium, potassium or lithium hydroxide in methanol, ethanol or dioxane at room temperature, or up to refluxing conditions) to provide compounds of formula (XV), as defined in formula I.

Compounds of formula (XXIV) may be converted to compounds XXV by rearrangement as displayed below, wherein #signifies the connection to residue G as defined in formula (I).

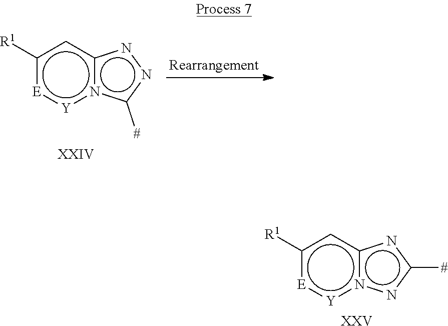

Reactions of this type have been described in Potts K. T., Surapaneni C. R., 1970, Journal of Heterocyclic Chemistry. or Nagamatsu T., Fujita T., 2002. Heterocycles. 57(4), 631-636. The reaction is typically carried out in the presence of a catalyst, usually an acid or a base, such as NaOH or formic acid, in an inert organic solvent or water at a temperature of from 0 to 80° C. If no catalyst is used, the reaction may be carried out at elevated temperatures, e.g. from 30 to 100° C.

Compounds of formula (XXIV) may be prepared by reaction of hydrazine compounds of formula (XXVI), as displayed under Process 8 below, wherein #signifies the connection to residue G as defined in formula (I).

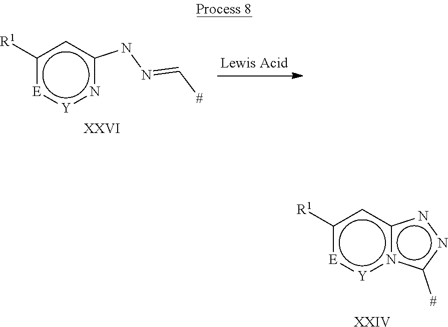

Reactions of this type have been described by Glushkov V. A. et al., 1998, Pharmaceutical Chemistry Journal, vol. 32(5), p. 29-32, or WO2012148808, p. 143. The reaction is typically performed in the presence of a Lewis acid, such as FeCl₃ or AlCl₃, at elevated temperatures of from 50 to 150° C. in an inert organic solvent. The reaction may be carried out in the presence of an oxidizing agent, e.g. H₂O₂ or CuCl₂.

Compounds of formula (XXVI) are accessible by reaction of hydrazine compounds of formula (XXVII) with aldehyde compounds of formula (XXVIII), as displayed under process 9 below.

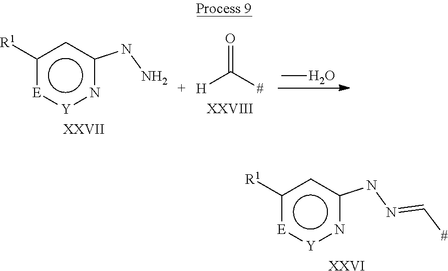

#signifies the connection to residue G as defined in formula (I).,

The reaction is typically carried out in the presence of an acid catalyst, such as toluene sulfonic acid, in an inert organic solvent. Suitable solvents are aliphatic hydrocarbons, such as pentane, hexane, cyclohexane, or petrol ether; or aromatic hydrocarbons, such as benzene, toluene, o-, m-, and p-xylene. Mixtures of the above solvents are also possible

Hydrazine derivatives of formula (XXVII) are commercially available or may be derived from commercially available compounds. Alternatively, compounds of formula (XXVII) may also be prepared by reaction of hydrazine with compound of formula (XXIX), as displayed under Process 10 below.

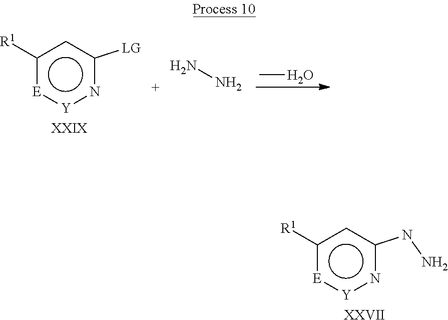

wherein LG is a leaving group, and wherein all other variables have a meaning as defined for formula (I). Typical leaving groups are triflate, iodide and chloride. Reactions of this type have been described in Mao, Y. et al, 2014, Journal of Heterocyclic Chemistry, 51(3), p. 594-597. The reaction is typically carried out in a polar solvent, such as CH₃CH₂OH under elevated temperatures, such as from 50 to 100° C. Compounds of formula (XXXV) are commercially available or can be prepared by standard methods of organic chemistry.

By analogy, compounds IIa, falling under the definition of formula III, can be prepared as displayed under Process 11 below.

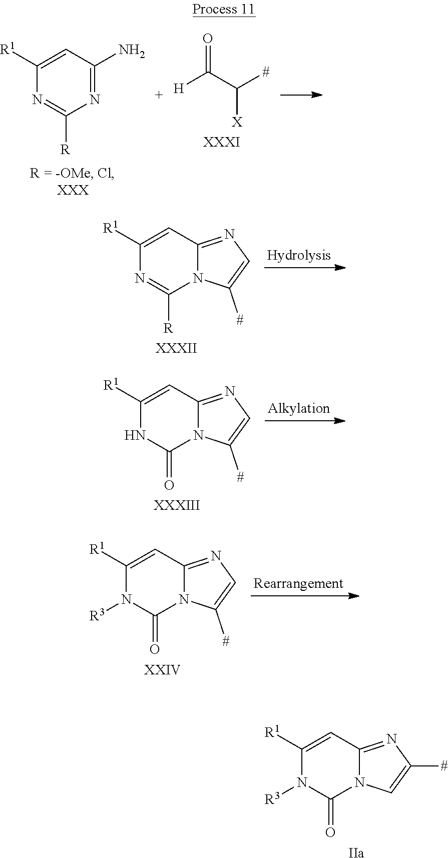

Compounds of formula (XXX) are commercially available or can be prepared by standard methods of organic chemistry. Condensation of compound XXX with aldehyde XXXI by an analogy to method described in Wade et. al. U.S. Pat. No. 4,503,050, 5 Mar. 1985 yield compounds XXXII. Hydrolysis of R (Cl, —OMe) present in compounds XXXII under the condition known in literature result in compounds XXVI. Compounds XXVI can be prepared from XXXIII by N-alkylation reaction under basic condition or copper catalyzed condition as described in Bioorganic & Medicinal Chemistry Letters, 25(6), 1310-1317; 2015. Rearrangement of XXIV as described under process 7 to give compound IIa.

Alternatively, compounds IIIa can be obtained as displayed under Scheme 2 below.

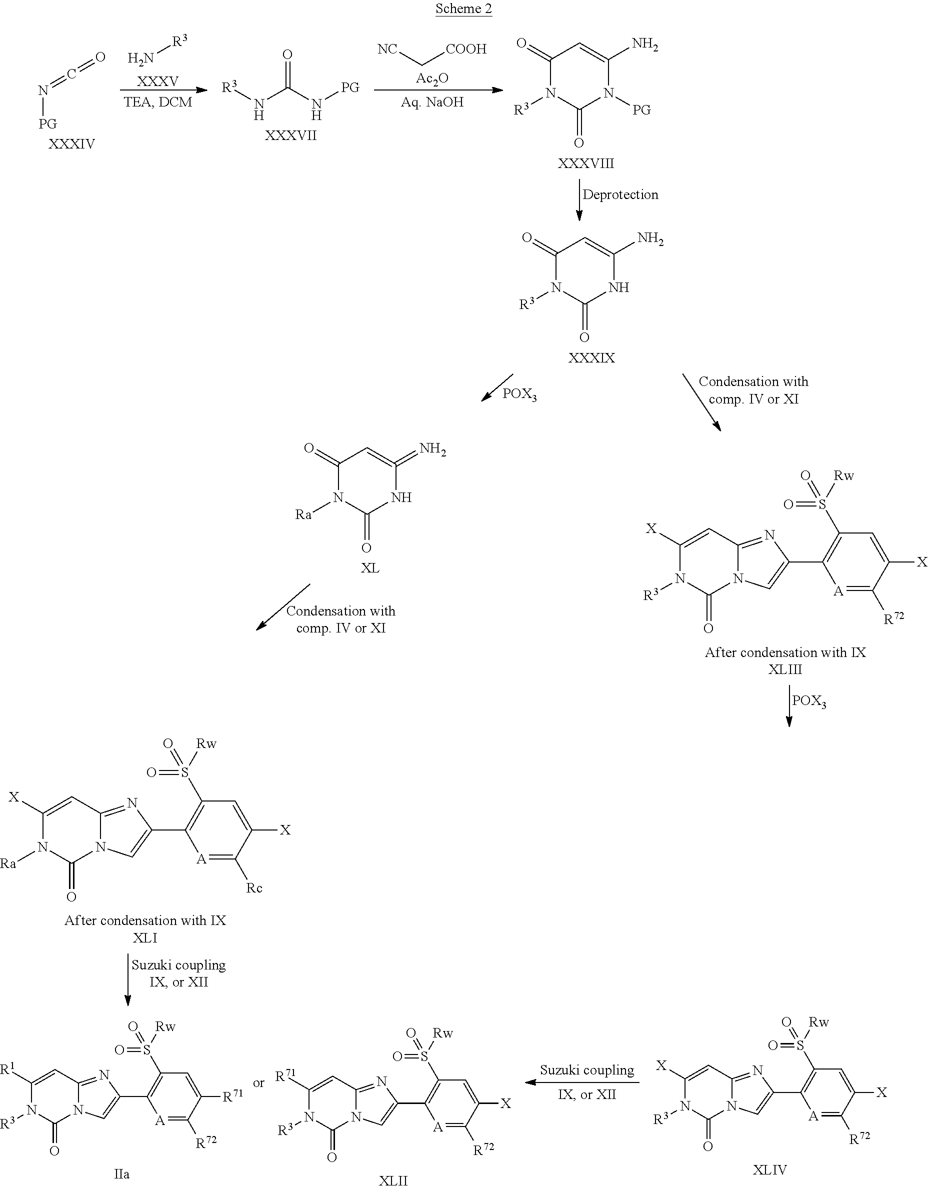

Compounds XXXVII, wherein PG signifies a protective group, can be obtained by reaction of commercially available compounds XXXIV with compounds XXV in the presence of an inert solvent, such as DCM, THF, DMF, or dioxane, and a base, such as triethylamine (TEA) at 20 to 100° C. as described in European Journal of Medicinal Chemistry (1990), 25(8), 653-8.

Compounds XXXVIII can be obtained by reacting compounds XXXVII with cyanoacetic acid in presence of acetic anhydride followed by reacting it under aqueous basic condition such as (aq. NaOH, aqueous KOH, aqueous Na₂CO₃, aqueous K₂CO₃ etc) as described in CN112125903 A and CN111170951.

Removal of the protecting group from the intermediate XXXVIII under basic condition (aq. NaOH, aqueous KOH, aqueous Na₂CO₃, aqueous K₂CO₃), acidic condition (aqueous HCl, aqueous H₂SO₄, AcOH) or under hydrogenation conditions in presence of H₂ and a metal catalyst such as Rh, Pd, Pt or by using transfer hydrogenation reaction using ammonium formate, potassium formate or sodium formate results in the formation of compounds XXXIX.

Compounds XL can be prepared from compounds XXXIX by reaction with POX₃ (e.g. POCl₃, POBr₃) in presence of polar aprotic solvent such as THF, dioxane, DEE etc as described in Tetrahedron Letters (2003), 44(13), 2717-2720. Further condensation of compounds XL with compounds IV or XI utilizing the processes as described in process 1 generates compounds XLI. Further Suzuki coupling reaction of XLI with IX or XII under standard suzuki type conditions described in process 1 result in compounds XLII and/or IIa.

Alternatively, compounds XXXIX can be condensed with intermediate IV or XI using the process describe under process I to get an intermediate XLIII. Compounds XLIV may then be prepared by converting the hydroxy group to R¹ wherein X is a leaving group such as Cl, Br, —OTf, —Onf, -tosyl, mesyl etc) using the processes known to a person skilled to the art. Further suzuki coupling of XLIV with IX or XII can generate compounds IIa and/or compounds XLII.

Compounds of formula (XLV), falling under the definition of formula (I), wherein R⁷¹ is a group —C(CN)(CH₃)₂, or 1-cyanocyclopropyl, may for example be prepared from compounds XLVI by condensing it with compounds XLVII (synthesized as described in WO2021204577) as per the procedure described under process 1 and as displayed under Process 13 below:

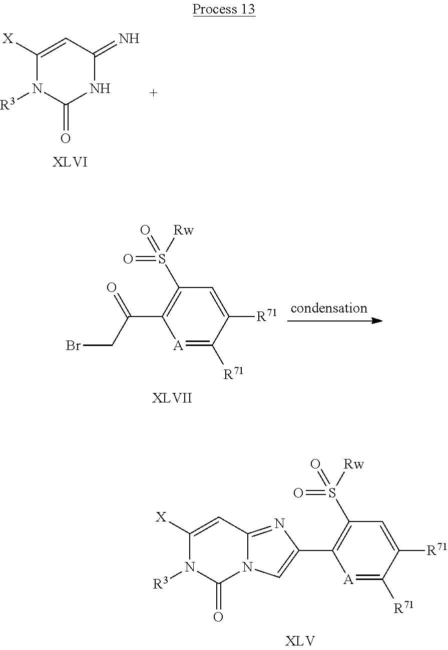

#signifies the connection to residue G as defined in formula (I).,

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

The N-oxides may be prepared from the inventive compounds according to conventional oxidation methods, e. g. by treating compounds I with an organic peracid such as metachloroperbenzoic acid (cf. WO 03/64572 or J. Med. Chem. 38(11), 1892-903, 1995); or with inorganic oxidizing agents such as hydrogen peroxide (cf. J. Heterocyc. Chem. 18(7), 1305-8, 1981) or oxone (cf. J. Am. Chem. Soc. 123(25), 5962-5973, 2001). The oxidation may lead to pure mono-N-oxides or to a mixture of different N-oxides, which can be separated by conventional methods such as chromatography.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

A skilled person will readily understand that the preferences for the variables defined herein, also in particular the ones given in the tables below for the respective substituents, given herein in connection with compounds I apply for the intermediates (compounds I to XLVII) accordingly. Thereby, the substituents in each case have independently of each other or more preferably in combination the meanings as defined herein.

The variables have, each on their own and in combination, the following preferred meanings. In one embodiment of compounds of formula (I), E is NR³ and Q is CH. In another embodiment of compounds of formula (I), E is NR³ and Q is N.

In one embodiment of the invention the compound of formula (I) is a compound of formula (I.A), or a compound (I. B).

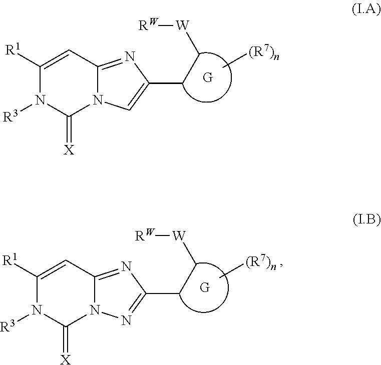

wherein all variables have a meaning as defined for formula (I). In one embodiment the compounds of formula (I) are compounds of formula (I.A). In another embodiment, the compounds of formula (I) are compounds of formula (I.B).

In one embodiment of the invention the compound of formula (I) is a compound of formula (I.A),
wherein
G is phenyl or pyridyl;
W is S, S(O), S(O)₂, S(O)(NRH);
R¹ is halogen;
R³ is C₁-C₆-alkyl, C₃-C₆-cycloalkyl, C₃-C₆-cycloalkyl-C₁-C₄-alkyl, which are unsubstituted or halogenated;
each R⁷ is independently H, halogen, OH, CN;
   C₁-C₆-alkyl, C₃-C₆-cycloalkyl, C₂-C₆-alkenyl, C₂-C₆-alkynyl, which groups are unsubstituted, or halogenated;
   a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more R^{H};
   phenyl, which is unsubstituted, or substituted with one or more R^{J};
   OR^{K};
   C(CN)(CH₃)₂, or 1-cyanocyclopropyl;
each R^{H} is independently halogen, CN; C₁-C₃-alkyl, C₁-C₃-haloalkyl;
each R^{J} is independently halogen, CN; C₁-C₆-alkyl, C₁-C₆-alkoxy, which groups are unsubstituted, or halogenated; or in case of two adjacent substituents R^{J} which are C₁-C₆-alkoxy, the substituents form an alkyl bridge between the two oxygens, which is unsubstituted or halogenated;
each R^{K} is C₁-C₆-alkyl which is unsubstituted or substituted with one or more halogen;
each R^{W} is independently C₁-C₆-alkyl;
the index n is 1 or 2;
and the N-oxides, stereoisomers, tautomers and agriculturally or veterinarily acceptable salts thereof.

In one embodiment of the invention the compound of formula (I) is a compound of formula (I.A), wherein
G is phenyl or pyridyl;
W is S(O)₂, S(O)(NRH);
R¹ is Cl or Br;
R³ is C₁-C₂-alkyl, cyclopropyl, or cyclopropylmethyl;
each R⁷ is independently H, chloro, bromo;
   C₁-C₂-alkyl, C₃-C₆-cycloalkyl, which groups are unsubstituted, or halogenated;
   pyridine which is unsubstituted, or substituted with one or more R^{H};
   phenyl, which is unsubstituted, or substituted with one or more R^{J};
   OR^{K};
   C(CN)(CH₃)₂, or 1-cyanocyclopropyl;
each R^{H} is independently halogen, CN; C₁-C₃-alkyl, C₁-C₃-haloalkyl;
each R^{J} is independently fluoro, chloro, CN; C₁-C₆-alkyl, C₁-C₆-alkoxy, which groups are unsubstituted, or halogenated; or in case of two adjacent substituents R^{J} which are C₁-C₆-alkoxy, the substituents form an alkyl bridge between the two oxygens, which is unsubstituted or halogenated;
each R^{K} is C₁-C₆-alkyl which is unsubstituted or substituted with one or more halogen;
R^{W} is ethyl or isopropyl;
the index n is 1 or 2;
and the N-oxides, stereoisomers, tautomers and agriculturally or veterinarily acceptable salts thereof.

In one embodiment of the invention the compound of formula (I) is a compound of formula (I.A), wherein
G is pyridyl;
W is S(O)₂;
R¹ is Cl;
R³ is methyl, cyclopropyl, or cyclopropylmethyl;
each R⁷ is independently H, chloro, bromo;
   C₁-C₂-alkyl, C₃-C₆-cycloalkyl, which groups are unsubstituted, or halogenated; 1,3-benzodioxol-5-yl, which is unsubstituted or substituted with halogen;
   pyridine which is unsubstituted, or substituted with one or more R^{H};
   phenyl, which is unsubstituted, or substituted with one or more R^{J};
   OR^{K};
   C(CN)(CH₃)₂, or 1-cyanocyclopropyl;
each R^{H} is independently halogen, CN; C₁-C₃-alkyl, C₁-C₃-haloalkyl;
each R^{J} is independently fluoro, chloro, CN; C₁-C₆-alkyl, C₁-C₆-alkoxy, which groups are unsubstituted, or halogenated; or in case of two adjacent substituents R^{J} which are C₁-C₆-alkoxy, the substituents form an alkyl bridge between the two oxygens, which is unsubstituted or halogenated;
each R^{K} is C₁-C₆-alkyl which is unsubstituted or substituted with one or more halogen;
R^{W} is ethyl or isopropyl;
the index n is 1 or 2;
and the N-oxides, stereoisomers, tautomers and agriculturally or veterinarily acceptable salts thereof.

In a preferred embodiment of the invention, the compound of formula (I) is a compound of formula (I.A.1)

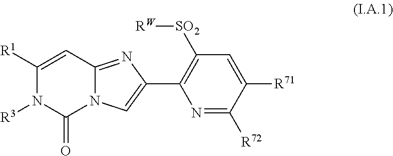

wherein the variables R¹, R³ and R^{W} have the meaning as defined for formula (I) or (I.A), and wherein the variables R⁷¹ and R⁷² have the meaning as defined for R⁷ in formula (I).

In another embodiment of the invention, the compound of formula (I) is a compound of formula (I.A.2)

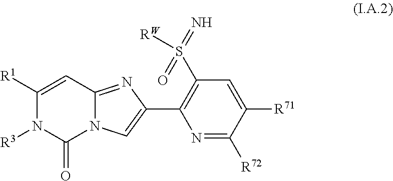

wherein the variables R¹, R³ and R^{W} have the meaning as defined for formula (I) or (I.A), and wherein the variables R⁷¹ and R⁷² have the meaning as defined for R⁷ in formula (I).

In another embodiment of the invention, the compound of formula (I) is a compound of formula (I.A.3)

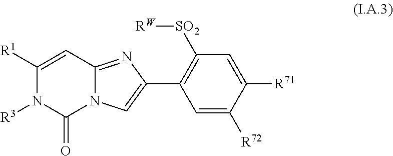

wherein the variables R¹, R³ and R^{W} have the meaning as defined for formula (I) or (I.A), and wherein the variables R⁷¹ and R⁷² have the meaning as defined for R⁷ in formula (I).

R¹ is halogen, i.e. F, Cl, Br, or I, preferably F, Cl, or Br, more preferably Cl or Br. In one embodiment, R¹ is Cl. In another embodiment, R¹ is Br.

R³, R⁵ are independently C₁-C₆-alkyl, C₁-C₆-alkoxy, C₂-C₆-alkenyl, C₂-C₆-alkynyl, C₃-C₆-cycloalkyl, C₃-C₆-cycloalkyl-C₁-C₄-alkyl, which are unsubstituted or halogenated. In one embodiment, R³, R⁵ are independently C₁-C₃-alkyl, or C₃-C₆ cycloalkyl, which groups are unsubstituted or halogenated. In another embodiment, R³, R⁵ are independently cyclopropyl. In one embodiment, R³ is C₁-C₂-alkyl, cyclopropyl, or cyclopropylmethyl. Preferably, R³ is cyclopropyl.

The cycle G is phenyl or pyridyl. In one embodiment, G is phenyl. In another embodiment, G is pyridyl, more preferably 2-pyridyl.

Preferred rings G are depicted below as formulae A1 to A3, wherein “&” stands for the connection to the trycyclic scaffold of compounds of formula (I). For the avoidance of doubt, the formulae A1 to A3 are preferred embodiments on their own and in combination for the following group

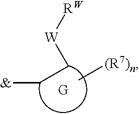

wherein “&” stands for the connection to the tricyclic scaffold in formula (I). In other words, the substituents R^{W}—W and (R⁷)ₙ in formulae A1 to A3 are mere illustrations but are not part of the rings G.

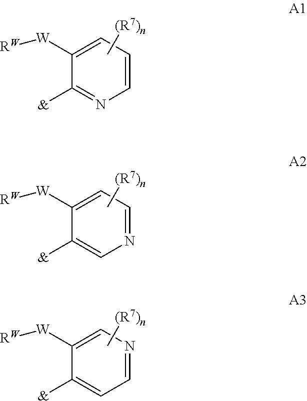

In one embodiment, G is A1. In another embodiment, G is A2. In another embodiment, G is A3. The variable W is S, S(O), S(O)₂ or S(O)(NR^{W}), preferably S(O)₂. In one embodiment, the variable W is S(O)(NH). In one embodiment, the variable W is not S(O)NH, whereas the other variables have the meaning as described in the embodiments herein.

The index n is 1, 2, 3, or 4, if G is phenyl, or 1, 2, or 3 if G is a pyridyl. Typically, n is 1 or 2. In another embodiment, n is 1. In another embodiment, n is 2. In another embodiment, n is 3.

In one embodiment, R⁷ is independently H, halogen, OH, CN; C₁-C₆-alkyl, C₃-C₆-cycloalkyl, C₂-C₆-alkenyl, C₂-C₆-alkynyl, which groups are unsubstituted, or halogenated; a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted, or substituted with one or more R^{H}; phenyl, which is unsubstituted, or substituted with one or more R^{J}; OR^{K}; —C(CN)(CH₃)₂, or 1-cyanocyclopropyl.

In another embodiment, R⁷ is halogen; C₁-C₃-alkyl, cyclopropyl; a 6- to 9-membered saturated, partially unsaturated, or fully unsaturated heterocyclic ring or ring system, wherein said heterocyclic ring or ring system comprises one or more, same or different heteroatoms O, or N, and is unsubstituted, or substituted with one or more substituents selected from halogen, CN, C₁-C₃-alkyl and C₁-C₃-haloalkyl; phenyl, which is unsubstituted, or substituted with one or more R^{J}; or OR^{K}, wherein R^{K} is C₁-C₃-alkyl, which is unsubstituted or substituted with CN.

In another embodiment, R⁷ is selected from —C(CN)(CH₃)₂, and 1-cyanocyclopropyl.

In another embodiment, R⁷ is halogen; CH₃, cyclopropyl; pyridyl, which is substituted with CN; 1,3-benzodioxol-5-yl, which is substituted with halogen; phenyl, which is substituted with halogen, or C₁-C₃-alkoxy, which is substituted with CN. In another embodiment, R⁷ is selected from —C(CN)(CH₃)₂, and 1-cyanocyclopropyl.

In another embodiment, R⁷ is halogen; CH₃, cyclopropyl; pyridyl, which is substituted with CN; 1,3-benzodioxol-5-yl, which is substituted with halogen; phenyl, which is substituted with halogen, C₁-C₃-alkoxy, which is substituted with CN, —C(CN)(CH₃)₂, or 1-cyanocyclopropyl.

In another embodiment, R⁷ is halogen; CH₃, cyclopropyl; pyridyl, which is substituted with CN; 1,3-benzodioxol-5-yl, which is substituted with halogen; phenyl, which is substituted with halogen, CN, C₁-C₆-alkyl, C₁-C₆-alkoxy, which groups are unsubstituted, or halogenated; C(CN)(CH₃)₂, or 1-cyanocyclopropyl.

In a preferred embodiment, R⁷ is chloro, bromo, 4-fluorophenyl, 2,4-difluorophenyl, 3,5-difluorophenyl, 2,6-dichlorophenyl, 4-chlorophenyl, methyl, cyclopropyl or phenyl substituted with C₁-C₂-alkyl, C₁-C₂-alkoxy, which groups are unsubstituted, or halogenated.

Each R^{H} is independently halogen, CN; C₁-C₃-alkyl, or C₁-C₃-haloalkyl. Preferably, each R^{H} is independently CN, or C₁-C₃-haloalkyl, more preferably CN or CF₃.

Each R^{J} is independently halogen, CN; C₁-C₃-alkyl, or C₁-C₃-alkoxy, which groups are unsubstituted or halogenated. Preferably, each R^{J} is independently halogen, CN, or C₁-C₃-haloalkoxy, more preferably halogen, CN, CF₃O, or CHF₂O, especially preferably halogen, CF₃O, or CHF₂O.

In one embodiment, there are two adjacent substituents R^{J} which are C₁-C₆-alkoxy, so that the substituents form an alkyl bridge between the two oxygens, which is unsubstituted or halogenated.

Examples for adjacent substituents R^{J} forming an alkylene ether bridge are as follows:

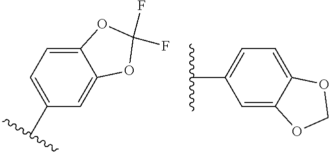

Each R^{K} is C₁-C₆-alkyl, C₂-C₆-alkenyl, C₂-C₆-alkynyl, C₃-C₆-cycloalkyl, which groups are unsubstituted or substituted with one or more, same or different substituents selected from halogen, and CN. Preferably, each R^{K} is C₁-C₃-alkyl, which is substituted with CN, more preferably isopropyl, which is substituted with CN, in particular 1-cyanoisopropyl.

R^{W} is C₁-C₄-alkyl, or cyclopropyl, preferably ethyl or isopropyl.

Formula I.A.1 is a preferred embodiment falling under the definition of formula IA, wherein n is 2, and wherein residues R⁷¹ and R⁷² have a definition according to R⁷.

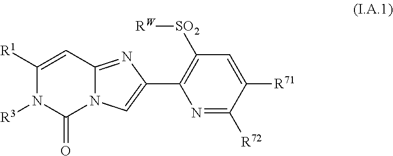

The following Table A represents specific compounds fall in combinations of definitions for substituents R¹ and R⁷¹ in lines L.1 to L.75. Each combination individually and all combinations collectively represent preferred embodiments. Each of the groups mentioned for a substituent in the tables is furthermore per se, independently of the combination in which it is mentioned, a particularly preferred aspect of the substituent in question.

Table A
Preferred combinations I.1 to 1.75 of R¹ and R⁷¹

| Line | R¹ | R⁷¹ | Line | R¹ | R⁷¹ |
|---|---|---|---|---|---|
| L. 1 | Cl | 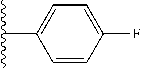 [SMILES: *c1ccc(F)cc1] | L. 13 | Br | 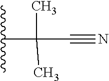 [SMILES: *C(C)(C)C#N] |
| L.2 | Cl | 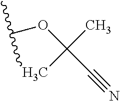 [SMILES: *OC(C)(C)C#N] | L. 14 | Br | 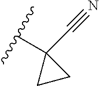 [SMILES: *C1(C#N)CC1] |
| L.3 | Cl | 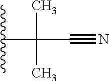 [SMILES: *C(C)(C)C#N] | L. 15 | Br | 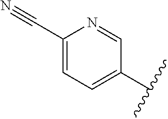 [SMILES: *c1ccc(C#N)nc1] |
| L.4 | Cl | 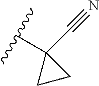 [SMILES: *C1(C#N)CC1] | L. 16 | Br | 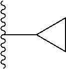 [SMILES: *C1CC1] |
| L.5 | Cl | 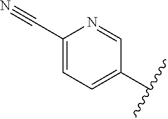 [SMILES: *c1ccc(C#N)nc1] | L. 17 | Br | 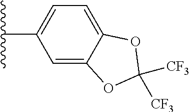 [SMILES: *c1ccc2c(c1)OC(C(F)(F)F)(C(F)(F)F)O2] |
| L.6 | Cl | 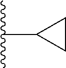 [SMILES: *C1CC1] | L. 18 | Br | 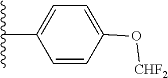 [SMILES: *c1ccc(OC(F)F)cc1] |
| L.7 | Cl | 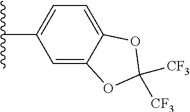 [SMILES: *c1ccc2c(c1)OC(C(F)(F)F)(C(F)(F)F)O2] | L. 19 | Br | Br |
| L.8 | Cl | 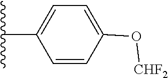 [SMILES: *c1ccc(OC(F)F)cc1] | L.20 | Br | 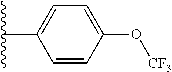 [SMILES: *c1ccc(OC(F)(F)F)cc1] |
| L.9 | Cl | Br | L.21 | Cl | 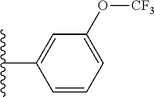 [SMILES: *c1cccc(OC(F)(F)F)c1] |
| L. 10 | Cl | 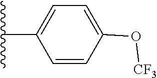 [SMILES: *c1ccc(OC(F)(F)F)cc1] | L.22 | Cl | 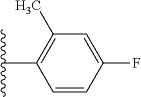 [SMILES: *c1ccc(F)cc1C] |
| L. 11 | Br | 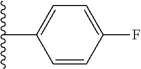 [SMILES: *c1ccc(F)cc1] | L.23 | Cl | 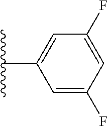 [SMILES: *c1cc(F)cc(F)c1] |
| L. 12 | Br | 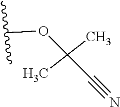 [SMILES: *OC(C)(C)C#N] | | | |
| L.24 | Br | 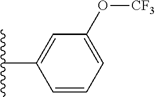 [SMILES: *c1cccc(OC(F)(F)F)c1] | L.35 | Br | 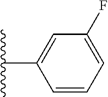 [SMILES: *c1cccc(F)c1] |
| L.25 | Br | 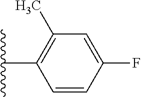 [SMILES: *c1ccc(F)cc1C] | L.36 | Cl | 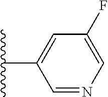 [SMILES: *c1cncc(F)c1] |
| L.26 | Cl | 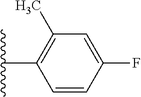 [SMILES: *c1ccc(F)cc1C] | L.37 | Br | 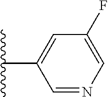 [SMILES: *c1cncc(F)c1] |
| L.27 | Cl | 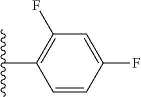 [SMILES: *c1ccc(F)cc1F] | L.38 | Cl | 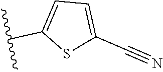 [SMILES: *c1ccc(C#N)s1] |
| L.28 | Br | 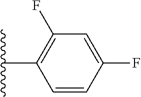 [SMILES: *c1ccc(F)cc1F] | L.39 | Br | 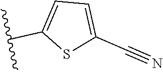 [SMILES: *c1ccc(C#N)s1] |
| L.29 | Cl | 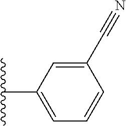 [SMILES: *c1cccc(C#N)c1] | L.40 | Cl | 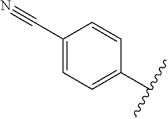 [SMILES: *c1ccc(C#N)cc1] |
| L.29 | Br | 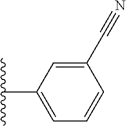 [SMILES: *c1cccc(C#N)c1] | L.41 | Br | 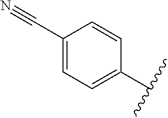 [SMILES: *c1ccc(C#N)cc1] |
| L.30 | Cl | 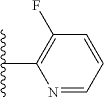 [SMILES: *c1ncccc1F] | L.42 | Cl | 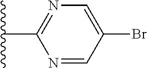 [SMILES: *c1ncc(Br)cn1] |
| L.31 | Br | 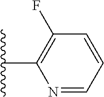 [SMILES: *c1ncccc1F] | L.43 | Br | 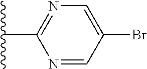 [SMILES: *c1ncc(Br)cn1] |
| L.32 | Cl | 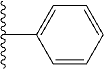 [SMILES: *c1ccccc1] | L.44 | Cl | 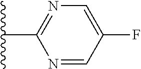 [SMILES: *c1ncc(F)cn1] |
| L.33 | Br | 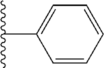 [SMILES: *c1ccccc1] | L.45 | Br | 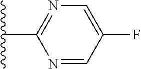 [SMILES: *c1ncc(F)cn1] |
| L.34 | Cl | 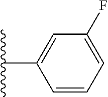 [SMILES: *c1cccc(F)c1] | L.46 | Cl | 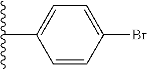 [SMILES: *c1ccc(Br)cc1] |
| | | | L. 47 | Br | 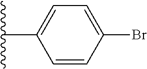 [SMILES: *c1ccc(Br)cc1] |
| L.48 | Cl | 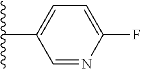 [SMILES: *c1ccc(F)nc1] | L.59 | Br | 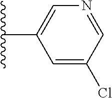 [SMILES: *c1cncc(Cl)c1] |
| L.49 | Br | 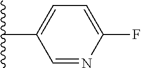 [SMILES: *c1ccc(F)nc1] | L.60 | Cl | 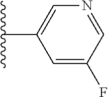 [SMILES: *c1cncc(F)c1] |
| L.50 | Cl | 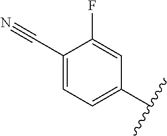 [SMILES: *c1ccc(C#N)c(F)c1] | L.61 | Br | 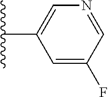 [SMILES: *c1cncc(F)c1] |
| L.51 | Br | 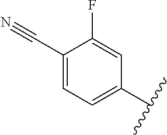 [SMILES: *c1ccc(C#N)c(F)c1] | L.62 | Cl | 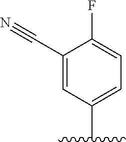 [SMILES: *c1ccc(F)c(C#N)c1] |
| L.52 | Cl | 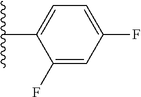 [SMILES: *c1ccc(F)cc1F] | L.63 | Br | 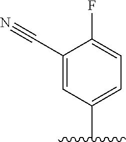 [SMILES: *c1ccc(F)c(C#N)c1] |
| L.53 | Br | 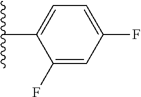 [SMILES: *c1ccc(F)cc1F] | L.64 | Cl | 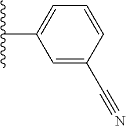 [SMILES: *c1cccc(C#N)c1] |
| L.54 | Cl | 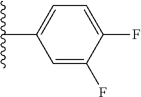 [SMILES: *c1ccc(F)c(F)c1] | L.65 | Br | 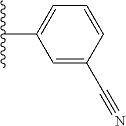 [SMILES: *c1cccc(C#N)c1] |
| L.55 | Br | 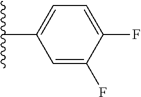 [SMILES: *c1ccc(F)c(F)c1] | L.66 | Cl | 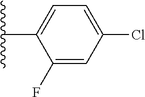 [SMILES: *c1ccc(Cl)cc1F] |
| L.56 | Cl | 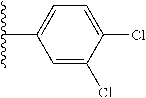 [SMILES: *c1ccc(Cl)c(Cl)c1] | L.67 | Br | 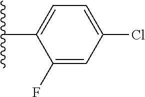 [SMILES: *c1ccc(Cl)cc1F] |
| L.57 | Br | 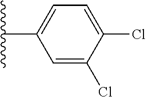 [SMILES: *c1ccc(Cl)c(Cl)c1] | | | |
| L.58 | Cl | 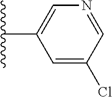 [SMILES: *c1cncc(Cl)c1] | | | |
| L.68 | Cl | 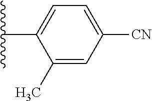 [SMILES: *c1ccc(C#N)cc1C] | L.72 | Cl | 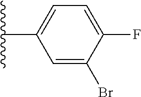 [SMILES: *c1ccc(F)c(Br)c1] |
| L.69 | Br | 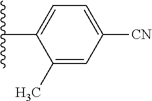 [SMILES: *c1ccc(C#N)cc1C] | L.73 | Br | 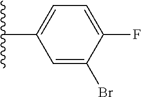 [SMILES: *c1ccc(F)c(Br)c1] |
| L.70 | Cl | 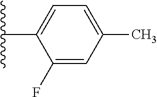 [SMILES: *c1ccc(C)cc1F] | L.74 | C | 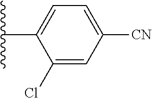 [SMILES: *c1ccc(C#N)cc1Cl] |
| L.71 | Br | 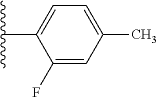 [SMILES: *c1ccc(C)cc1F] | L.75 | Br | 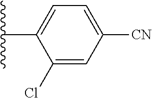 [SMILES: *c1ccc(C#N)cc1Cl] |

Especially preferred embodiments of the present invention are listed in Tables 1 to 4, corresponding to specific compounds falling under formula I.A.

Table 1: a compound of formula I.A.1, wherein R³ is cyclopropyl, R^{W} is ethyl, R⁷² is H, and the combination of R¹ and R⁷¹ is as defined in a line of Table A.

Table 2: a compound of formula I.A.1, wherein R³ is cyclopropyl, R^{W} is ethyl, R⁷² is CH₃, and the combination of R¹ and R⁷¹ is as defined in a line of Table A.

Table 3: a compound of formula I.A.1, wherein R³ is cyclopropyl, R^{W} is ethyl, R⁷² is CH₂CH₃, and the combination of R¹ and R⁷¹ is as defined in a line of Table A.

Table 4: a compound of formula I.A.1, wherein R³ is cyclopropyl, R^{W} is ethyl, R⁷² is Cl, and the combination of R¹ and R⁷¹ is as defined in a line of Table A.

Table 5: a compound of formula I.A.1, wherein R³ is cyclopropyl, R^{W} is ethyl, R⁷² is Br, and the combination of R¹ and R⁷¹ is as defined in a line of Table A.

Table 6: a compound of formula I.A.1, wherein R³ is cyclopropyl, R^{W} is ethyl, R⁷² is cyclopropyl, and the combination of R¹ and R⁷¹ is as defined in a line of Table A.

Table 7: a compound of formula I.A.1, wherein R³ is cyclopropyl, R^{W} is ethyl, R⁷² is isopropyl, and the combination of R¹ and R⁷¹ is as defined in a line of Table A.

Table 8: a compound of formula I.A.1, wherein R³ is cyclopropyl, R^{W} is ethyl, R⁷² is N-imidazolyl, and the combination of R¹ and R⁷¹ is as defined in a line of Table A.

Table 9: a compound of formula I.A.1, wherein R³ is cyclopropyl, R^{W} is ethyl, R⁷² is N-triazolyl, and the combination of R¹ and R⁷¹ is as defined in a line of Table A.

Table 10: a compound of formula I.A.1, wherein R³ is cyclopropyl, R^{W} is isopropyl, R⁷² is H, and the combination of R¹ and R⁷¹ is as defined in a line of Table A.

Table 11: a compound of formula I.A.1, wherein R³ is cyclopropyl, R^{W} is isopropyl, R⁷² is CH₃, and the combination of R¹ and R⁷¹ is as defined in a line of Table A.

Table 12: a compound of formula I.A.1, wherein R³ is cyclopropyl, R^{W} is isopropyl, R⁷² is CH₂CH₃, and the combination of R¹ and R⁷¹ is as defined in a line of Table A.

Table 13: a compound of formula I.A.1, wherein R³ is cyclopropyl, R^{W} is isopropyl, R⁷² is Cl, and the combination of R¹ and R⁷¹ is as defined in a line of Table A.

Table 14: a compound of formula I.A.1, wherein R³ is cyclopropyl, R^{W} is isopropyl, R⁷² is Br, and the combination of R¹ and R⁷¹ is as defined in a line of Table A.

Table 15: a compound of formula I.A.1, wherein R³ is cyclopropyl, R^{W} is isopropyl, R⁷² is cyclopropyl, and the combination of R¹ and R⁷¹ is as defined in a line of Table A.

Table 16: a compound of formula I.A.1, wherein R³ is cyclopropyl, R^{W} is isopropyl, R⁷² is isopropyl, and the combination of R¹ and R⁷¹ is as defined in a line of Table A.

Table 17: a compound of formula I.A.1, wherein R³ is cyclopropyl, R^{W} is isopropyl, R⁷² is N-imidazolyl, and the combination of R¹ and R⁷¹ is as defined in a line of Table A.

Table 18: a compound of formula I.A.1, wherein R³ is cyclopropyl, R^{W} is isopropyl, R⁷² is N-triazolyl, and the combination of R¹ and R⁷¹ is as defined in a line of Table A.

In a preferred embodiment, the compound of formula (I) is a compound of formula (I.A) wherein
G is pyridyl,
R¹ is halogen;
R³ is cyclopropyl;
R^{W} is C₁-C₄-alkyl, cycloalkyl;
n is 1 or 2;
R⁷ is selected from
   halogen;
   C₁-C₃-alkyl, cyclopropyl;
   a 6- to 9-membered saturated, partially unsaturated, or fully unsaturated heterocyclic ring or ring system, wherein said heterocyclic ring or ring system comprises one or more, same or different heteroatoms O, or N, and is unsubstituted, or substituted with one or more substituents selected from halogen, CN, C₁-C₃-alkyl, and C₁-C₃-haloalkyl;
   phenyl, which is unsubstituted, or substituted with one or more R^{J}; or
   OR^{K}, wherein R^{K} is C₁-C₃-alkyl, which is unsubstituted or substituted with CN;
   —C(CN)(CH₃)₂, and 1-cyanocyclopropyl.

In another preferred embodiment, the compound of formula (I) is a compound of formula (I.A) wherein G is pyridyl,
R¹ is halogen;
R³ is cyclopropyl;
R^{W} is C₁-C₄-alkyl, cycloalkyl;
n is 1 or 2;
R⁷ is phenyl with two adjacent substituents R^{J} which are C₁-C₆-alkoxy, wherein the two R^{J} form an alkyl bridge between the two oxygens, which is unsubstituted or halogenated.

In another preferred embodiment, the compound of formula (I) is a compound of formula (I.A) wherein G is pyridyl,
R¹ is halogen;
R³ is cyclopropyl;
R^{W} is C₁-C₃-alkyl;
n is 1 or 2;
R⁷ is selected from
   halogen; CH₃, cyclopropyl; pyridyl, which is substituted with CN; 1,3-benzodioxol-5-yl, which is substituted with halogen; phenyl, which is substituted with halogen, C₁-C₃-alkoxy, which is substituted with CN, —C(CN)(CH₃)₂, or 1-cyanocyclopropyl.

The term “compound(s) of the invention” refers to compound(s) of formula I, or “compound(s) I”, and includes their salts, tautomers, stereoisomers, and N-oxides.

The invention also relates to agrochemical compositions comprising an auxiliary and at least one compound I.

An agrochemical composition comprises a pesticidally effective amount of a compound I.

The compounds I can be converted into customary types of agro-chemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials e.g. seeds (e.g. GF). These and further compositions types are defined in the “Catalogue of pesticide formulation types and international coding system”, Technical Monograph No. 2, 6th Ed. May 2008, CropLife International. The compositions are prepared in a known manner, e.g. described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents. Suitable solid carriers or fillers are mineral earths.

Suitable surfactants are surface-active compounds, e.g. anionic, cationic, nonionic, and amphoteric surfactants, block polymers, polyelectrolytes. Such surfactants can be used as emulsifier, dispersant, solubilizer, wetter, penetration enhancer, protective colloid, or adjuvant. Surfactants are listed in McCutcheon's, Vol. 1: Emulsifiers & Detergents, McCutcheon's Directories, Glen Rock, USA, 2008 (International or North American Ed.). Suitable anionic surfactants are alkali, alkaline earth, or ammonium salts of sulfonates, sulfates, phosphates, carboxylates. Suitable nonionic surfactants are alkoxylates, N-substituted fatty acid amides, amine oxides, esters, sugar-based surfactants, polymeric surfactants. Suitable cationic surfactants are qua-ternary surfactants.

The agrochemical compositions generally comprise between 0.01 and 95%, preferably between 0.1 and 90%, and most preferably between 0.5 and 75%, by weight of active substance. The active substances are employed in a purity of from 90% to 100%, preferably from 95% to 100%. Various types of oils, wetters, adjuvants, or fertilizer may be added to the active substances or the compositions comprising them as premix or, if appropriate not until immediately prior to use (tank mix). These agents can be admixed with the compositions according to the invention in a weight ratio of 1:100 to 100:1.

The user applies the composition according to the invention usually from a predosage device, a knapsack sprayer, a spray tank, a spray plane, or an irrigation system. Usually, the agro-chemical composition is made up with water, buffer, and/or further auxiliaries to the desired application concentration and the ready-to-use spray liquor or the agrochemical composition according to the invention is thus obtained. Usually, 20 to 2000 liters, of the ready-to-use spray liquor are applied per hectare of agricultural useful area.

The compounds I are suitable for use in protecting crops, plants, plant propagation materials, e.g. seeds, or soil or water, in which the plants are growing, from attack or infestation by animal pests. Therefore, the invention also relates to a plant protection method, which comprises contacting crops, plants, plant propagation materials, e.g. seeds, or soil or water, in which the plants are growing, to be protected from attack or infestation by animal pests, with a pesticidally effective amount of a compound I.

The compounds I are also suitable for use in combating or controlling animal pests. Therefore, the invention also relates to a method of combating or controlling animal pests, which comprises contacting the animal pests, their habitat, breeding ground, or food supply, or the crops, plants, plant propagation materials, e.g. seeds, or soil, or the area, material or environment in which the animal pests are growing or may grow, with a pesticidally effective amount of a compound I.

The compounds I are effective through both contact and ingestion to any and all developmental stages, such as egg, larva, pupa, and adult.

The compounds I can be applied as such or in form of compositions comprising them.

The application can be carried out both before and after the infestation of the crops, plants, plant propagation materials by the pests.

The term “contacting” includes both direct contact (applying the compounds/compositions directly on the animal pest or plant) and indirect contact (applying the compounds/compositions to the locus).

The term “animal pest” includes arthropods, gastropods, and nematodes. Preferred animal pests according to the invention are arthropods, preferably insects and arachnids, in particular insects.

The term “plant” includes cereals, e.g. durum and other wheat, rye, barley, triticale, oats, rice, or maize (fodder maize and sugar maize/sweet and field corn); beet, e.g. sugar beet, or fodder beet; fruits, e.g. pomes, stone fruits, or soft fruits, e.g. apples, pears, plums, peaches, nectarines, almonds, cherries, papayas, strawberries, raspberries, blackberries or gooseberries; leguminous plants, e.g. beans, lentils, peas, alfalfa, or soybeans; oil plants, e.g. rapeseed (oilseed rape), turnip rape, mustard, olives, sunflowers, coconut, cocoa beans, castor oil plants, oil palms, ground nuts, or soybeans; cucurbits, e.g. squashes, pumpkins, cucumber or melons; fiber plants, e.g. cotton, flax, hemp, or jute; citrus fruit, e.g. oranges, lemons, grape-fruits or mandarins; vegetables, e.g. eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, e.g. avocados, cinnamon, or camphor; energy and raw material plants, e.g. corn, soybean, rapeseed, sugar cane or oil palm; tobacco; nuts, e.g. walnuts; pistachios; coffee; tea; bananas; vines; hop; sweet leaf (Stevia); natural rubber plants or ornamental and forestry plants, shrubs, broad-leaved trees or evergreens, Eucalyptus; turf; lawn; grass. Preferred plants include potatoes sugar beets, tobacco, wheat, rye, barley, oats, rice, corn, cotton, soybeans, rapeseed, legumes, sunflowers, coffee, or sugar cane; fruits; vines; ornamentals; or vegetables, e.g. cucumbers, tomatoes, beans or squashes.

The term “seed” embraces seeds and plant propagules including true seeds, seed pieces, suckers, corms, bulbs, fruit, tubers, grains, cuttings, cut shoots, and means preferably true seeds.

“Pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions e.g. desired pesticidal effect and duration, weather, target species, locus, mode of application.

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare.

The compounds I are also suitable for use against non-crop insect pests. For use against said non-crop pests, compounds I can be used as bait composition, gel, general insect spray, aero-sol, as ultra-low volume application and bed net (impregnated or surface applied).

The term “non-crop insect pest” refers to pests, which are particularly relevant for non-crop targets, e.g. ants, termites, wasps, flies, ticks, mosquitoes, bed bugs, crickets, or cockroaches, such as: Aedes aegypti, Musca domestica, Tribolium spp.; termites such as Reticulitermes flavipes, Coptotermes formosanus; roaches such as Blatella germanica, Periplaneta Americana; ants such as Solenopsis invicta, Linepithema humile, and Camponotus pennsylvanicus.

The bait can be a liquid, a solid or a semisolid preparation (e.g. a gel). For use in bait compositions, the typical content of active ingredient is from 0.001 wt % to 15 wt %, desirably from 0.001 wt % to 5 wt % of active compound.

The compounds I and its compositions can be used for protecting wooden materials such as trees, board fences, sleepers, frames, artistic artifacts, etc. and buildings, but also construction materials, furniture, leathers, fibers, vinyl articles, electric wires and cables etc. from ants, termites and/or wood or textile destroying beetles, and for controlling ants and termites from doing harm to crops or human beings (e.g. when the pests invade into houses and public facilities or nest in yards, orchards or parks).

Customary application rates in the protection of materials are, e.g., from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 wt %, preferably from 0.1 to 45 wt %, and more preferably from 1 to 25 wt % of at least one repellent and/or insecticide.

### Pests

The compounds of the invention are especially suitable for efficiently combating animal pests e.g. arthropods, and nematodes including:
insects from the sub-order of Auchenorrhyncha, e.g. Amrasca biguttula, Empoasca spp., Nephotettix virescens, Sogatella furcifera, Mahanarva spp., Laodelphax striatellus, Nilaparvata lugens, Diaphorina citri;
Lepidoptera, e.g. Helicoverpa spp., Heliothis virescens, Lobesia botrana, Ostrinia nubilalis, Plutella xylostella, Pseudoplusia includens, Scirpophaga incertulas, Spodoptera spp., Trichoplusia ni, Tuta absoluta, Cnaphalocrocis medialis, Cydia pomonella, Chilo suppressalis, Anticarsia gemmatalis, Agrotis ipsilon, Chrysodeixis includens;
True bugs, e.g. Lygus spp., Stink bugs such as Euschistus spp., Halyomorpha halys, Nezara viridula, Piezodorus guildinii, Dichelops furcatus;
Thrips, e.g. Frankliniella spp., Thrips spp., Dichromothrips corbettii; Aphids, e.g. Acyrthosiphon pisum, Aphis spp., Myzus persicae, Rhopalosiphum spp., Schizaphis graminum, Megoura viciae;
Whiteflies, e.g. Trialeurodes vaporariorum, Bemisia spp.;
Coleoptera, e.g. Phyllotreta spp., Melanotus spp., Meligethes aeneus, Leptinotarsa decimlineata, Ceutorhynchus spp., Diabrotica spp., Anthonomus grandis, Atomaria linearia, Agriotes spp., Epilachna spp.;
Flies, e.g. Delia spp., Ceratitis capitate, Bactrocera spp., Liriomyza spp.;
Coccoidea, e.g. Aonidiella aurantia, Ferrisia virgate;
Anthropods of class Arachnida (Mites), e.g. Penthaleus major, Tetranychus spp.;
Nematodes, e.g. Heterodera glycines, Meloidogyne spp., Pratylenchus spp., Caenorhabditis elegans.

In one embodiment, the pests are selected from Phyllophaga spp, Limonius spp, Popillia japonica.

### Animal Health

The compounds I are suitable for use in treating or protecting animals against infestation or infection by parasites. Therefore, the invention also relates to the use of a compound of the invention for the manufacture of a medicament for the treatment or protection of animals against infestation or infection by parasites. Furthermore, the invention relates to a method of treating or protecting animals against infestation and infection by parasites, which comprises orally, topically or parenterally administering or applying to the animals a parasiticidally effective amount of a compound I.

The invention also relates to the non-therapeutic use of compounds of the invention for treating or protecting animals against infestation and infection by parasites. Moreover, the invention relates to a non-therapeutic method of treating or protecting animals against infestation and infection by parasites, which comprises applying to a locus a parasiticidally effective amount of a compound I.

The compounds of the invention are further suitable for use in combating or controlling parasites in and on animals. Furthermore, the invention relates to a method of combating or controlling parasites in and on animals, which comprises contacting the parasites with a parasitically effective amount of a compound I.

The invention also relates to the non-therapeutic use of compounds I for controlling or combating parasites. Moreover, the invention relates to a non-therapeutic method of combating or controlling parasites, which comprises applying to a locus a parasiticidally effective amount of a compound I.

The compounds I can be effective through both contact (via soil, glass, wall, bed net, carpet, blankets or animal parts) and ingestion (e.g. baits). Furthermore, the compounds I can be applied to any and all developmental stages.

The compounds I can be applied as such or in form of compositions comprising them.

The term “locus” means the habitat, food supply, breeding ground, area, material or environment in which a parasite is growing or may grow outside of the animal.

As used herein, the term “parasites” includes endo- and ectoparasites. In some embodiments of the invention, endoparasites can be preferred. In other embodiments, ectoparasites can be preferred. Infestations in warm-blooded animals and fish include lice, biting lice, ticks, nasal bots, keds, biting flies, muscoid flies, flies, myiasitic fly larvae, chiggers, gnats, mosquitoes and fleas.

The compounds of the invention are especially useful for combating the following parasites: Cimex lectularius, Rhipicephalus sanguineus, and Ctenocephalides felis.

As used herein, the term “animal” includes warm-blooded animals (including humans) and fish. Preferred are mammals, such as cattle, sheep, swine, camels, deer, horses, pigs, poultry, rabbits, goats, dogs and cats, water buffalo, donkeys, fallow deer and reindeer, and also in furbearing animals such as mink, chinchilla and raccoon, birds such as hens, geese, turkeys and ducks and fish such as fresh- and salt-water fish such as trout, carp and eels. Particularly preferred are domestic animals, such as dogs or cats.

The compounds I may be applied in total amounts of 0.5 mg/kg to 100 mg/kg per day, preferably 1 mg/kg to 50 mg/kg per day.

For oral administration to warm-blooded animals, the compounds I may be formulated as animal feeds, animal feed premixes, animal feed concentrates, pills, solutions, pastes, suspensions, drenches, gels, tablets, boluses and capsules. For oral administration, the dosage form chosen should provide the animal with 0.01 mg/kg to 100 mg/kg of animal body weight per day of the compounds I, preferably with 0.5 mg/kg to 100 mg/kg of animal body weight per day. Alternatively, the compounds I may be administered to animals parenterally, e.g., by intraruminal, intramuscular, intravenous or subcutaneous injection. The compounds I may be dispersed or dissolved in a physiologically acceptable carrier for subcutaneous injection. Alternatively, the compounds I may be formulated into an implant for subcutaneous administration. In addition the compounds I may be transdermally administered to animals. For parenteral administration, the dosage form chosen should provide the animal with 0.01 mg/kg to 100 mg/kg of animal body weight per day of the compounds I.

The compounds I may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds I. In addition, the compounds I may be formulated as ear tags for animals, particularly quadrupeds e.g. cattle and sheep.

Oral solutions are administered directly.

Solutions for use on the skin are trickled on, spread on, rubbed in, sprinkled on or sprayed on. Gels are applied to or spread on the skin or introduced into body cavities.

Pour-on formulations are poured or sprayed onto limited areas of the skin, the active compound penetrating the skin and acting systemically. Pour-on formulations are prepared by dissolving, suspending or emulsifying the active compound in suitable skin-compatible solvents or solvent mixtures.

Emulsions can be administered orally, dermally or as injections.

Suspensions can be administered orally or topically/dermally.

Semi-solid preparations can be administered orally or topically/dermally.

For the production of solid preparations, the active compound is mixed with suitable excipients, if appropriate with addition of auxiliaries, and brought into the desired form.

The compositions which can be used in the invention can comprise generally from about 0.001 to 95% of the compound I.

Ready-to-use preparations contain the compounds acting against parasites, preferably ectoparasites, in concentrations of 10 ppm to 80% by weight, preferably from 0.1 to 65% by weight, more preferably from 1 to 50% by weight, most preferably from 5 to 40% by weight. Preparations which are diluted before use contain the compounds acting against ectoparasites in concentrations of 0.5 to 90% by weight, preferably of 1 to 50% by weight.

Furthermore, the preparations comprise the compounds of formula I against endoparasites in concentrations of 10 ppm to 2% by weight, preferably of 0.05 to 0.9% by weight, very particularly preferably of 0.005 to 0.25% by weight.

Solid formulations which release compounds of the invention may be applied in total amounts of 10 mg/kg to 300 mg/kg, preferably 20 mg/kg to 200 mg/kg, most preferably 25 mg/kg to 160 mg/kg body weight of the treated animal in the course of three weeks.

The following examples illustrate the invention.

### A. PREPARATION OF COMPOUNDS

Materials: Unless otherwise noted, reagents and solvents were purchased at highest commercial quality and used without further purification.

All reactions were monitored by thin-layer chromatography (TLC) using Merck silica gel 60 F₂54 pre-coated plates (0.25 mm). Flash chromatography was carried out with Agela technologies silica gel (Agela techno, silica irregular 40-60 μm, 60A, Cat.-No. C-CS1400).

¹H NMR spectra were recorded on Bruker (500 MHz). Chemical shifts are expressed in ppm downfield from the internal solvent peaks for DMSO-d₆ (¹H; δ=2.50 ppm) (¹H; δ=2.50 ppm) and CD₃OD (¹H; δ=3.30 ppm), and J values are given in Hertz. The following abbreviations were used to explain the multiplicities: s=singlet, d=doublet, t=triplet, q=quartet, dd=double doublet, dt=double triplet, m=multiplet, br=broad. High-resolution mass spectra were measured on a JEOL JMS-T100LP.

Characterization: The compounds were characterized by coupled High Performance Liquid Chromatography with mass spectrometry (HPLC/MS).

Method A: UHPLC-MS on Shimadzu LCMS 2020 ESI. Analytical UHPLC column: C-18, 50 mm, 4.6 mm, 5 micron; mobile phase: 100 mM Ammonium Formate, B: Acetonitrile, Flow Rate: 1.2 mL/min, Injection Vol: 1 μL in 1.50 min; Gradient: 10% B to 100% B in 1.5 min, Hold 100% B for 1 min, 2.51 min 10% B Run time: 3 min at 400° C. MS-method: ESI positive; mass range (m/z) 100-800.

Method B: Waters (ACQUITY-H Class UPLC. Analytical UHPLC column), Mass detector 3100: BEH-C-18, 50 mm, 2.1 mm, 1.7 micron; mobile phase: A: 0.1% formic acid, 10% IPA in Water. B: 0.1% formic acid in Acetonitrile C: 20 mM Ammonium Formate in water. Flow Rate: 1.2 mL/min, Injection Vol: 1 μL in 1.50 minutes; Gradient: 10% A to 100% B in 1.5 min, Hold 100% B for 1 min, 2.51 min 10% B Run time: 3 min at 400° C. MS-method: ESI positive; mass range (m/z) 100-800.

Abbreviations used: min is minutes; ACN is acetonitrile; DCM is dichlormethane; DMF is dimethylformamide; THF is tetrahydrofuran; mL is milliliters; min is minutes; h is hour(s)

### Synthesis Example 1: Manufacture of 7-chloro-6-cyclopropyl-2-[3-ethylsulfonyl-5-(4-fluorophenyl)-2-pyridyl]imidazo[1,2-c]pyrimidin-5-one (compound I.1)

### Step-1: Manufacture of 5-bromo-3-ethylsulfanyl-pyridine-2-carbonitrile

To a stirred solution of 5-bromo-3-nitro-pyridine-2-carbonitrile (20 g, 0.087 mol) in DMF (200 mL) at −40° C., was added sodium ethane thiolate (11 g, 0.105 mol) portion wise over a period of 30 min under N₂— atmosphere at maintained temperature between −40 to −50° C. The resultant reaction mixture was stirred at the same temperature for 10 minutes then gradually allowed to reach a temperature of 20 to 25° C. with continued stirring for the next 1 h. After the completion of reaction, above reaction mixture was quenched, extracted, and the organic layer was washed. The combined organic layers were dried, concentrated under reduced pressure to get crude mass, which was purified by column chromatography to afford 5-bromo-3-ethylsulfanyl-pyridine-2-carbonitrile as a yellow solid (17 g, 82% yield). H¹ NMR (500 MHz, CDCl₃): δ 8.52 (s, 1H), 7.85 (s, 1H), 3.07 (q, 2H, J=10 Hz), 1.43 (t, 3H, J=7.3 Hz). LC-MS: mass calculated for [M+H]⁺ 243, found 244.

### Step-2: Manufacture of 1-(5-bromo-3-ethylsulfanyl-2-pyridyl)ethanone

To a stirred solution of 5-bromo-3-ethylsulfanyl-pyridine-2-carbonitrile (17 g, 0.069 mol) in THF (170 mL) at 0° C., was added methyl magnesium bromide (2 eq) dropwise over a period of 30 min at 0° C. to −5° C. under N₂-atmosphere. The resultant reaction mixture was allowed to stir at 0° C. for 2 h. After the completion of reaction, the above reaction mixture was quenched with 1N aqueous solution of HCl (250 mL) and extracted with ethyl acetate. The organic layers were washed, dried, and concentrated under reduced pressure to get crude mass, which was purified by crystallization. Obtained solid was filtered and dried under reduced pressure to afford 1-(5-bromo-3-ethylsulfanyl-2-pyridyl)ethanone as a yellow solid (14 g, 82% yield). H¹ NMR (500 MHz, DMSO): δ 8.57 (s, 1H), 8.05 (s, 1H), 3.01 (q, 2H, J=10 Hz), 2.59 (s, 3H), 1.26 (t, 3H, J=7.3 Hz). LC-MS: mass calculated for [M+H]⁺ 260, found 261.

### Step-3: Manufacture of 1-(5-bromo-3-ethylsulfonyl-2-pyridyl)ethanone

To a stirred solution of 1-(5-bromo-3-ethylsulfanyl-2-pyridyl)ethanone (3.9 g, 0.018 mol) in DCM (40 mL) at 0° C., was added m-chloroperoxy benzoic acid (9.7 g, 0.039 mol). The resultant reaction mixture was stirred at 20 to 25° C. for 3-4 h. After the completion of reaction, the above reaction mixture was quenched, and extracted. The organic layer was washed, dried and concentrated under reduced pressure to get 1-(5-bromo-3-ethylsulfonyl-2-pyridyl)ethanone (3.2 g, 82% yield) as an off white solid. H¹ NMR (500 MHz, CDCl₃): δ 9.12 (s, 1H), 8.55 (s, 1H), 3.55 (q, 2H J=12 Hz), 2.5 (s, 3H), 1.20 (t, 3H, J=7 Hz). LC-MS: mass calculated for [M+H]⁺ 291, found 292.

### Step-4: Manufacture of 1-[3-ethylsulfonyl-5-(4-fluorophenyl)-2-pyridyl]ethanone

To a stirred solution of 1-(5-bromo-3-ethylsulfonyl-2-pyridyl)ethenone (10 gm, 0.342 mol) in dioxane (95 ml) was added 4-fluoro phenyl boronic acid (4.7 gm, 0.342 mol) and stirred for few minutes until a clear solution is obtained. To the reaction mass 5 ml H₂O was added followed by K₂CO₃ (14.919 gm, 1.026 mol) and was allowed to stir for 5 min under continuous N₂-purging via a gas injection. The reaction mass was finally added Pd(PPh₃)₄ (1.977 gm, 0.0171 mol). The reaction was then allowed to stir for a period of 2 hours at 100° C. After completion of the reaction, the above reaction mass was cooled, quenched, and extracted. The organic layer was then washed, dried and concentrated under reduced pressure to get a crude mass, which was purified by column to afford 1-[3-ethylsulfonyl-5-(4-fluorophenyl)-2-pyridyl]ethanone as an off white solid (8 g, 82% yield). ¹H NMR (300 MHz, DMSO-d₆) δ 9.25 (d, J=2.1 Hz, 1H), 8.52 (d, J=2.1 Hz, 1H), 8.02-7.89 (m, 2H), 7.49-7.35 (m, 2H), 3.60 (q, J=7.4 Hz, 2H), 2.54 (s, 3H), 1.25 (t, J=7.4 Hz, 3H). LC-MS: mass calculated for C₁₅H₁₄FNO₃S [M]⁺ 307, found 308.

### Step-5: Manufacture of 2-bromo-1-[3-ethylsulfonyl-5-(4-fluorophenyl)-2-pyridyl]ethanone

To a stirred solution of 1-[3-ethylsulfonyl-5-(4-fluorophenyl)-2-pyridyl]ethanone (3 g, 0.010 mol) in chloroform (30 mL) at 0° C., was added CH₃COOH (30 mL) and HBr in acetic acid (30 mL) and stirred for few minutes. Then followed by the addition of bromine (2 g 0.012 mol) in CHCl₃. The resultant reaction mixture was heated to 60° C. for 1 h. After the completion of the reaction, the above reaction mixture was quenched, extracted, and the organic layer was washed. The combined organic layers were dried and concentrated under reduced pressure to get crude mass, which was purified by column chromatography to afford 2-bromo-1-[3-ethylsulfonyl-5-(4-fluorophenyl)-2-pyridyl]ethanone as an off white solid (2.2 g, 82% yield). ¹H NMR (300 MHz, DMSO-d₆) δ 9.23 (d, J=2.1 Hz, 1H), 8.61 (m, 1H), 7.92 (ddd, J=8.7, 5.3, 2.7 Hz, 2H), 7.43-7.29 (m, 2H), 4.90 (s, 2H), 3.57 (q, J=7.4 Hz, 2H), 1.18 (t, J=7.4 Hz, 3H). LC-MS: mass calculated for C₁₅H₁₃BrFNO₃S [M]⁺ 386, found 386.1.

### Step-6: Manufacture of 1-benzyl-3-cyclopropyl-urea

To a solution of cyclopropylamine (16 g, 0.281 mol, 1.5 eq) and triethyl amine (51 mL, 0.375 mol, 2 eq) in DCM (dichloromethane) (1 L) was added a solution of benzyl isocyanate (25 g, 0.187 mol, 1 eq) in DCM (dichloromethane) (100 mL) at 0° C. The reaction mixture was gradually warmed to 20 to 25° C. and was stirred for 1 hour. After completion of reaction, it was diluted with DCM until a clear solution was observed. The organic layer was treated with aqueous 1M HCl (500 ml) followed by saturated aqueous NaHCO₃ (500 mL). The combined organic extract was washed with water and brine. It was then dried, and concentrated to afford 1-benzyl-3-cyclopropyl-urea as a white solid (35 g, 97%).

¹H NMR (300 MHz, DMSO-d₆) δ 7.37-7.16 (m, 5H), 6.39 (t, J=6.1 Hz, 1H), 6.21 (d, J=2.6 Hz, 1H), 4.22 (d, J=6.1 Hz, 2H), 2.43 (tq, J=6.8, 3.4 Hz, 1H), 0.67-0.49 (m, 2H), 0.40-0.29 (m, 2H). LC-MS: mass calculated for C₁₁H₁₄N₂O [M]⁺ 190, found 191.

### Step-7: Manufacture of 6-amino-1-benzyl-3-cyclopropyl-pyrimidine-2,4-dione

To a stirred solution 1-benzyl-3-cyclopropyl-urea (65 g, 0.3684 mol 1 eq) and cyanoacetic acid (37.57 g, 0.4421 mol, 1.2 eq) was added acetic anhydride slowly at 0° C. Resultant reaction mixture was stirred at 100° C. for 3-4 h. After completion of the reaction, the above reaction mixture was quenched and extracted. The organic layer was washed with water, dried and concentrated under reduced pressure to afford an intermediate, which was purified by column chromatography to afford the desired intermediate (45 gm, 51%). The intermediate was then taken in 10 volume of water and subsequently treated with an aqueous solution of 2 N NaOH (4 Volume) at 20 to 25° C. The resulting reaction mixture was stirred at 20 to 25° C. for 30 min, precipitated white solid were separated, filtered through Buckner funnel, obtained solid cake was washed with water and was dried under reduced pressure to afford 6-amino-1-benzyl-3-cyclopropyl-pyrimidine-2,4-dione as white solid (42 g, 47%).

¹H NMR (500 MHz, Chloroform-d) δ 7.45-7.31 (m, 4H), 7.31-7.25 (m, 1H), 5.19 (s, 2H), 5.01 (s, 1H), 2.74 (tt, J=7.1, 4.0 Hz, 1H), 1.22-1.10 (m, 2H), 0.94-0.83 (m, 2H). LC-MS: mass calculated for C₁₄H₁₅N₃O₂ [M]⁺ 257, found 258.

### Step-8: Manufacture of 6-amino-3-cyclopropyl-1H-pyrimidine-2,4-dione

To a stirred solution of 6-amino-1-benzyl-3-cyclopropyl-pyrimidine-2,4-dione (7 g, 0.0272 mol, 1 eq.) in CH₃OH (350 mL) at 25° C. potassium formate (22.87 g, 0.2723 mol, 10 eq) was added to observe a clear solution followed by 10% Pd/C (1.44 gm, 0.05 eq) at 20 to 25° C. maintaining an inert atmosphere. Resultant reaction mixture was heated to 70° C. for 1.5 h. After the consumption of starting material was observed by TLC, the reaction mass was allowed to elevate to 50° C. after which reaction the mixture was filtered through celite bed at 50° C. The celite bed was subsequently washed with hot CH₃OH (50° C.), the filtrate containing the reaction mass was concentrated to dryness, to which concentrate was added 10 vol. water. Subsequently, the pH was adjusted in the range of 5 to 6 with 6 N HCl at at a temperature of 0° C. The obtained solid was filtered and washed with water to afford 6-amino-3-cyclopropyl-1H-pyrimidine-2,4-dione as white solid (80 to 90% yield). ¹H NMR (500 MHz, DMSO-d₆) δ 10.10 (s, 1H), 6.03 (s, 1H), 4.42 (d, J=1.7 Hz, 1H), 2.33 (td, J=7.1, 3.6 Hz, 1H), 0.79 (t, J=6.9 Hz, 2H), 0.55 (p, J=5.2 Hz, 2H). LC-MS: mass calculated for C₇H₉N₃O₂ [M]⁺ 167, found 168.

### Step-9: Manufacture of 6-amino-4-chloro-3-cyclopropyl-1,6-dihydropyrimidin-2-one

To 6-amino-3-cyclopropyl-1H-pyrimidine-2,4-dione (0.5 gm) was added POCl₃ (1 mL) at 20 to 25° C. slowly, the rection mixture was then gradually heated to 130° C. and stirred for 12 h.

Reaction was concentrated under reduced pressure to remove POCl₃, upon which the reaction mixture was slowly added into 4 volume ice water, adjusted (pH=10) by 6 N aqueous solution of NaOH to precipitate the solid. The solid was separated, filtered through buckner funnel, washed with water, evaporated to dryness to afford 6-amino-4-chloro-3-cyclopropyl-1,6-dihydropyrimidin-2-one as yellow solid (60% yield). ¹H NMR (500 MHz, DMSO-d₆) δ 10.45 (s, 1H), 7.13 (d, J=2.1 Hz, 1H), 3.57 (d, J=2.1 Hz, 1H), 2.92 (s, 1H), 0.84 (s, 2H), 0.64 (s, 2H). LC-MS: mass calculated for C₇H₈ClN₃O [M]⁺ 185, found 186.

### Step-10: Manufacture of 7-chloro-6-cyclopropyl-2-[3-ethylsulfonyl-5-(4-fluorophenyl)-2-pyridyl]imidazo[1,2-c]pyrimidin-5-one (compound I.1)

To a stirred solution of 6-amino-4-chloro-3-cyclopropyl-1,6-dihydropyrimidin-2-one (1 g, 5.34 mmol) in tert-butanol (10 mL) was added 2-bromo-1-[2-ethylsulfonyl-4-(4-fluorophenyl)phenyl]ethanone (2.04 g, 5.34 mmol). Molecular sieves were added to the above reaction mixture (1 g) and the resultant reaction mixture was heated to 95° C. for 16 h. After the completion of the reaction, the above reaction mixture was filtered through celite bed, the filtrate was collected and concentrated under reduced pressure to get a crude mass. The crude mass was purified by column chromatography to afford 7-chloro-6-cyclopropyl-2-[2-ethylsulfonyl-4-(4-fluorophenyl)phenyl]imidazo[1,2-c]pyrimidin-5-one as a yellow solid (0.75 g, 30.08% yield).

¹H NMR (300 MHz, DMSO-d₆)¹H NMR (300 MHz, DMSO-d₆) b 9.25 (d, J=2.2 Hz, 1H), 8.54 (d, J=2.2 Hz, 1H), 8.15 (s, 1H), 7.99-7.89 (m, 2H), 7.42 (t, J=8.8 Hz, 2H), 7.12 (s, 1H), 4.08 (q, J=7.4 Hz, 2H), 3.09 (s, 1H), 1.25 (m, 5H), 1.06 (m, 2H). LC-MS: mass calculated for C₂₂H₁₈ClFN₄O₃S [M]⁺ 472, found 473.

### Synthesis example 2: Manufacture of 7-bromo-6-cyclopropyl-2-[3-ethylsulfonyl-5-(4-fluorophenyl)-2-pyridyl]imidazo[1,2-c]pyrimidin-5-one (compound I.4)

To a stirred solution of 7-chloro-6-cyclopropyl-2-[3-ethylsulfonyl-5-(4-fluorophenyl)-2-pyridyl]imidazo[1,2-c]pyrimidin-5-one (compound I.1, 0.6 g, 1.2 mmol) in CH₃COOH (6 mL) was added HBr in CH₃COOH (0.6 mL, 1.9 mmol), and the resultant reaction mixture was heated to 120° C. for 16 h. After the completion of starting material the reaction mixture was poured into water (100 mL) and extracted with ethyl acetate. The organic layer was separated, dried, and concentrated to get a crude mass. The crude mass was then purified by column chromatography to afford 7-bromo-6-cyclopropyl-2-[3-ethylsulfonyl-5-(4-fluorophenyl)-2-pyridyl]imidazo[1,2-c]pyrimidin-5-one as a beige solid (0.5 g 75% yield). 1H NMR (500 MHz, DMSO-d₆) δ 9.26-9.22 (m, 1H), 8.54 (t, J=1.9 Hz, 1H), 8.15 (s, 1H), 7.97-7.91 (m, 2H), 7.42 (td, J=8.0, 7.3, 1.8 Hz, 2H), 7.26 (s, 1H), 4.08 (q, J=7.4 Hz, 2H), 3.25 (s, 1H), 1.25 (m, 3H), 1.21 (m, 2H), 1.06 (m, 2H). LC-MS: mass calculated for C₂₂H₁₈BrFN₄O₃S [M]⁺ 517.3, found 519.2 [M+2].

### Synthesis example 3: Manufacture of 7-chloro-6-cyclopropyl-2-[3-ethylsulfonyl-5-(4-fluorophenyl)-6-methyl-2-pyridyl]imidazo[1,2-c]pyrimidin-5-one (compound I.5)

### Step-1: Manufacture of 5-fluoro-2-methyl-pyridin-3-amine

To a stirred solution of 2-bromo-5-fluoro-pyridin-3-amine (15 g, 78.53 mmol), trimethylboroxine 50% in THF (39.4 mL, 157.1 mmol) and K₂CO₃ (27.13 g, 196.734 mmol) in 1,4-dioxane (120 mL) and H₂O (20 mL) were degassed under N₂-atmosphere for 15 min and followed by the addition of [1,1′-bis(diphenylphosphino)ferrocene]dichloropalladium(II), complex with CH₂Cl₂ (3.02 g, 3.92 mmol). The resultant reaction mixture was heated to 120° C. for 6-7 h. After the completion of the reaction, the reaction mixture was poured into water and extracted with ethyl acetate. The organic layer was separated, dried, and concentrated to get a crude mass. The crude mass was purified by column chromatography to afford 5-fluoro-2-methyl-pyridin-3-amine (7 g 60% yield). ¹H NMR (500 MHz, DMSO-d₆) δ: 8.15 (dd, J=8.1, 1.9 Hz, 1H), 8.11 (dd, J=8.1, 1.9 Hz, 1H), 3.61 (q, J=7.3 Hz, 2H), 2.37 (s, 3H). LC-MS: mass calculated for C₆H₇FN₂ [M]⁺ 126.1, found 127.5 [M+1].

### Step-2: Manufacture of 6-bromo-5-fluoro-2-methyl-pyridin-3-amine

To a stirred solution of 5-fluoro-2-methyl-pyridin-3-amine (9 g, 71.35 mmol), in CH₂Cl₂ (120 mL) at 0° C. was added N-bromosuccinimide (12.7 g, 71.35 mmol). The resultant reaction mixture was stirred at the same temperature for 10 min. After the completion of the reaction, the reaction mixture was poured into water and extracted with CH₂Cl₂. The organic layer was washed with an aqueous saturated solution of sodium thiosulphate, the separated organic layer was dried, and concentrated to get a crude mass. The crude mass was purified by column chromatography to afford 6-bromo-5-fluoro-2-methyl-pyridin-3-amine (9 g, 60% yield). ¹H NMR (500 MHz, DMSO-d₆) δ: 8.11 (dd, J=8.1, 1.9 Hz, 1H), 3.61 (q, J=7.3 Hz, 2H), 2.37 (s, 3H). LC-MS: mass calculated for C₆H₆BrFN₂ [M]⁺ 205, found 207 [M+2].

### Step-3: Manufacture of 1-(5-amino-3-fluoro-6-methyl-2-pyridyl)ethanone

To a stirred solution of 6-bromo-5-fluoro-2-methyl-pyridin-3-amine (9 g, 2.03 mmol) and tributyl(1-ethoxyvinyl)tin (23.78 mL, 2.03 mmol) in toluene (10 V, 100 mL) was degassed under N₂-atmosphere for 10 min and followed with addition of tetrakis-triphenylphosphin-palladium (2.53 g, 32.19 mmol). The resultant reaction mixture was heated to 120° C. for 6-7 h. After the completion of the reaction mixture, the reaction mixture was quenched with water and extracted with ethyl acetate. The organic layer was separated, dried, and concentrated to get a crude mass. The crude mass was purified by column chromatography to afford 1-(5-amino-3-fluoro-6-methyl-2-pyridyl)ethanone (5 g 50% yield). ¹H NMR (500 MHz, DMSO-d₆) δ: 8.11 (dd, J=8.1, 1.9 Hz, 1H), 3.61 (q, J=7.3 Hz, 2H), 2.37 (s, 3H), 1.15-1.08 (m, 3H). LC-MS: mass calculated for C₃H₉FN₂O [M]⁺ 168, found 168.1.

### Step-4: Manufacture of 1-(5-bromo-3-fluoro-6-methyl-2-pyridyl)ethanone

To a stirred solution of 1-(5-amino-3-fluoro-6-methyl-2-pyridyl)ethanone (4.2 g, 24.97 mmol), in ACN (50 mL) at 0° C. was added Cu(I)Br (3.58 g, 24.97 mmol) and isopentyl nitrite (4.38 g, 37.46 mmol). The resultant reaction mixture was stirred at the same temperature for 10 minutes, then stirred at 20 to 25° C. for 1 h. After the completion of the reaction, the reaction mixture was filtered through celite pad, the filtrate was poured into H₂O and extracted. The organic layer was separated, dried, and concentrated to get a crude mass. The crude mass was purified by column chromatography to afford 1-(5-bromo-3-fluoro-6-methyl-2-pyridyl)ethanone (2.5 g 40% yield). ¹H NMR (500 MHz, DMSO-d₆) δ: 8.11 (dd, J=8.1, 1.9 Hz, 1H), 2.37 (s, 3H), 1.15-1.08 (m, 3H). LC-MS: mass calculated for C₃H₇BrFNO [M]⁺ 232, found 234 [M+2].

Step 5: Manufacture of 1-[3-fluoro-5-(4-fluorophenyl)-6-methyl-2-pyridyl]ethanone A stirred solution of 1-(5-bromo-3-fluoro-6-methyl-2-pyridyl)ethanone (1.5 g, 6.46 mmol), (4-fluorophenyl)boronic acid (1.35 g, 9.69 mmol) and K₂CO₃ (1.78 g, 12.92 mmol) in 1,4-dioxane (10 mL) and water (2 mL) was degassed under N₂-atmosphere for 15 min, followed by the addition of [1,1′-bis(diphenylphosphino)ferrocene]dichloropalladium(II), complex with DCM (0.373 g, 0.323 mmol). The resultant reaction mixture was heated to 120° C. for 6 h. After the completion of the reaction, the reaction mixture was poured into water and extracted. The organic layer was separated, dried, and concentrated to get a crude mass. The crude mass was purified by column chromatography to afford 1-[3-fluoro-5-(4-fluorophenyl)-6-methyl-2-pyridyl]ethanone (0.9 g 66% yield). ¹H NMR (300 MHz, Chloroform-d) δ 8.15 (s, 1H), 7.46-7.05 (m, 4H), 2.63 (s, 3H), 1.53 (s, 3H). LC-MS: mass calculated for C₁₄H₁₁F₂NO [M]⁺ 247.2, found 248.1 [M+1].

### Step-6: Manufacture of 1-[3-ethylsulfonyl-5-(4-fluorophenyl)-6-methyl-2-pyridyl]ethanone

To a stirred solution of 1-[3-fluoro-5-(4-fluorophenyl)-6-methyl-2-pyridyl]ethanone (0.8 g, 3.39 mmol) and sodium ethanesulfonate (0.898 g, 6.79 mmol) in dimethylacetamide (10 mL). The resultant reaction mixture was heated to 120° C. for 2 h. After the completion of the reaction, the reaction mixture was diluted with water and extracted. The organic layer was separated, dried, and concentrated to get a crude mass. The crude mass was purified by column chromatography to afford 1-[3-ethylsulfonyl-5-(4-fluorophenyl)-6-methyl-2-pyridyl]ethanone as a pale yellow solid (0.88 g 95% yield). ¹H NMR (300 MHz, Chloroform-d) δ 8.15 (s, 1H), 7.46-7.05 (m, 4H), 3.59 (q, J=7.5 Hz, 2H), 2.63 (s, 3H), 1.53 (s, 3H), 1.36 (t, J=7.5 Hz, 3H). LC-MS: mass calculated for C₁₆H₁₆FNO₃S [M]⁺ 321.3, found 322.1 [M+1].

### Step-7: Manufacture of 2-bromo-1-[3-ethylsulfonyl-5-(4-fluorophenyl)-6-methyl-2-pyridyl]ethanone

To a stirred solution of 1-[3-ethylsulfonyl-5-(4-fluorophenyl)-6-methyl-2-pyridyl]ethanone (0.9 g, 3.39 mmol) and pyridinumbromide-perbromide (0.94 g, 6.79 mmol) in dioxane (10 mL). The resultant reaction mixture was heated to 90° C. for 1.5 h. After the completion of the reaction, the reaction mixture was diluted with ice cold water (100 mL) and extracted. The organic layer was separated, dried and concentrated to get a crude mass. The crude mass was purified by column chromatography to afford 2-bromo-1-[3-ethylsulfonyl-5-(4-fluorophenyl)-6-methyl-2-pyridyl]ethanone as a pale yellow solid (0.74 g 60% yield). ¹H NMR (300 MHz, Chloroform-d) δ 8.15 (s, 1H), 7.46-7.05 (m, 4H), 4.96 (s, 2H), 3.59 (q, J=7.5 Hz, 2H), 2.63 (s, 3H), 1.36 (t, J=7.5 Hz, 3H). LC-MS: mass calculated for C₁₆H₁₅BrFNO₃S [M]⁺ 400.2, found 402.0 [M+2].

### Step-8: Manufacture of 7-chloro-6-cyclopropyl-2-[3-ethylsulfonyl-5-(4-fluorophenyl)-6-methyl-2-pyridyl]imidazo[1,2-c]pyrimidin-5-one

To a stirred solution of 4-amino-6-chloro-1-cyclopropyl-pyrimidin-2-one (0.4 g, 2.155 mmol) in tert-butanol (2 mL) was added 2-bromo-1-[3-ethylsulfonyl-5-(4-fluorophenyl)-6-methyl-2-pyridyl]ethanone (1.03 g, 2.58 mmol). Molecular sieves were added to the above reaction mixture (0.1 g) and the resultant reaction mixture was heated to 100° C. for 24 h. After the completion of the reaction, the above reaction mixture was filtered through celite bed, the filtrate was collected and concentrated under reduced pressure to get crude mass, which was purified by column chromatography to afford 7-chloro-6-cyclopropyl-2-[3-ethylsulfonyl-5-(4-fluorophenyl)-6-methyl-2-pyridyl]imidazo[1,2-c]pyrimidin-5-one as a yellow solid (0.15 g, 30% yield). ¹H NMR (300 MHz, DMSO-d₆) δ 8.11 (d, J=8.9 Hz, 2H), 7.61 (dd, J=8.5, 5.5 Hz, 2H), 7.39 (t, J=8.8 Hz, 2H), 7.12 (s, 1H), 4.01 (q, J=7.4 Hz, 2H), 2.58 (s, 3H), 1.22 (m, 6H), 1.06 (s, 2H). LC-MS: mass calculated for C₂₃H₂ClFN₄O₃S [M]⁺ 486.9, found 487.1.

With appropriate modification of the starting materials or intermediates thereof, the procedures as described in the preparation example above were used to obtain further compounds of formulae I.A.1 listed in Table B with their physical data.

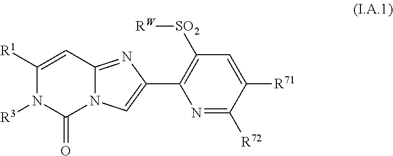

TABLE B

| No. | R¹ | R³ | R⁷¹ | R⁷² | R^{W} | ¹H-NMR data (δ); LCMS [RT (min), M⁺] |
|---|---|---|---|---|---|---|
| 1.1 | Cl | cyclo-propyl | 4-fluorophenyl | H | CH₃CH₂ | (300 MHz, DMSO-d₆) δ 9.25 (d, J = 2.2 Hz, 1H), 8.54 (d, |
| | | | | | | J = 2.2 Hz, 1H), 8.15 (s, 1H), 7.99-7.89 (m, 2H), 7.42 (t, |
| | | | | | | J = 8.8 Hz, 2H), 7.12 (s, 1H), 4.08 (q, J = 7.4 Hz, 2H), 3.09 |
| | | | | | | (s, 1H), 1.25 (m, 5H), 1.06 (m, 2H). LC-MS: mass |
| | | | | | | calculated for C₂₂H₁₈ClFN₄O₃S [M]⁺ 472, found 473. |
| 1.2 | Cl | cyclopropyl | 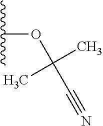 [SMILES: *OC(C)(C)C#N] | H | CH₃CH₂ | 1H NMR (300 MHz, DMSO-d6) δ 8.81 (d, J = 2.7 Hz, 1H), 8.20 (d, J = 2.7 Hz, 1H), 8.10 (s, 1H), 7.11 (s, 1H), 4.05 (q, J = 7.4 Hz, 2H), 3.07 (dq, J = 7.0, 3.5 Hz, 1H), 1.82 (s, 6H), 1.22 (m, 5H), 1.16-0.99 (m, 2H). LC-MS: mass calculated for C₂₀H₂₀ClN₅O₄S [M]⁺ 461, found 462 [M + 1]. |
| 1.3 | Cl | cyclopropyl | 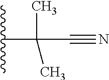 [SMILES: *C(C)(C)C#N] | H | CH₃CH₂ | 1H NMR (300 MHz, DMSO-d6) δ 9.12 (d, J = 2.3 Hz, 1H), 8.45 (d, J = 2.3 Hz, 1H), 8.14 (s, 1H), 7.12 (d, J = 0.8 Hz, 1H), 4.06 (q, J = 7.3 Hz, 2H), 3.15-3.03 (m, 1H), 1.84 (s, 6H), 1.24 (m, 5H), 1.07 (d, J = 7.8 Hz, 2H). LC-MS: mass calculated for C₂₀H₂₀ClN₅O₃S [M]⁺ 445.9, found 446 [M+]. |
| 1.4 | Br | cyclopropyl | 4-fluorophenyl | H | CH₃CH₂ | (500 MHz, DMSO-d₆) δ 9.26-9.22 (m, 1H), 8.54 (t, J = |
| | | | | | | 1.9 Hz, 1H), 8.15 (s, 1H), 7.97-7.91 (m, 2H), 7.42 (td, J = |
| | | | | | | 8.0, 7.3, 1.8 Hz, 2H), 7.26 (s, 1H), 4.08 (q, J = 7.4 Hz, 2H), |
| | | | | | | 3.25 (s, 1H), 1.25 (m, 3H), 1.21 (m, 2H), 1.06 (m, 2H). LC- |
| | | | | | | MS: mass calculated for C₂₂H₁₈BrFN₄O₃S [M]⁺ 517.3, |
| | | | | | | found 519.2 [M + 2]. |
| 1.5 | Cl | cyclopropyl | 4-fluorophenyl | CH3 | CH₃CH₂ | (300 MHz, DMSO-d₆) δ 8.11 (d, J = 8.9 Hz, 2H), 7.61 (dd, |
| | | | | | | J = 8.5, 5.5 Hz, 2H), 7.39 (t, J = 8.8 Hz, 2H), 7.12 (s, 1H), |
| | | | | | | 4.01 (q, J = 7.4 Hz, 2H), 2.58 (s, 3H), 1.22 (m, 6H), 1.06 |
| | | | | | | (s, 2H). LC-MS: mass calculated for C₂₃H₂₀ClFN₄O₃S |
| | | | | | | [M]⁺ 486.9, found 487.1. |
| 1.6 | Cl | cyclopropyl | 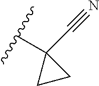 [SMILES: *C1(C#N)CC1] | H | CH₃CH₂ | (300 MHz, DMSO-d6) δ 8.82 (d, J = 2.3 Hz, 1H), 8.33 (d, J = 2.3 Hz, 1H), 8.11 (s, 1H), 7.11 (s, 1H), 4.05 (q, J = 7.3 Hz, 2H), 3.20-2.96 (m, 1H), 2.04-1.88 (m, 2H), 1.88- 1.73 (m, 2H), 1.23 (m, 5H), 1.05 (d, J = 4.3 Hz, 2H). LC- MS: mass calculated for C₂₀H₁₈ClN₅O₃S [M]⁺ 443.9, found 444.3. |
| 1.7 | Cl | cyclopropyl | 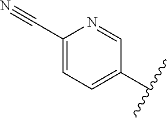 [SMILES: *c1ccc(C#N)nc1] | H | CH₃CH₂ | (300 MHz, DMSO-d6) ? 9.38 (d, J = 2.2 Hz, 1H), 9.29 (d, J = 2.3 Hz, 1H), 8.72 (d, J = 2.2 Hz, 1H), 8.59 (dd, J = 8.2, 2.4 Hz, 1H), 8.35-8.12 (m, 2H), 7.13 (s, 1H), 4.10 (q, J = 7.4 Hz, 2H), 3.20-2.97 (m, 1H), 1.26 (m, 5H), 1.07 (m, 2H). LC-MS: mass calculated for C₂₂H₁₇ClN₆O₃S [M]⁺ 480.9, found 481.2. |
| 1.8 | Cl | cyclopropyl | 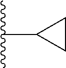 [SMILES: *C1CC1] | H | CH₃CH₂ | (300 MHz, DMSO-d₆) δ 8.69 (d, J = 2.1 Hz, 1H), 8.07- 7.98 (m, 2H), 7.08 (d, J = 0.7 Hz, 1H), 3.96 (q, J = 7.4 Hz, 2H), 3.07 (dq, J = 7.3, 3.7 Hz, 1H), 2.18 (td, J = 8.5, 4.3 Hz, 1H), 1.27-1.10 (m, 6H), 1.15-1.07 (m, 1H), 1.04 (s, |
| | | | | | | 2H), 1.01-0.84 (m, 2H). LC-MS: mass calculated for |
| | | | | | | C₁₉H₁₉ClFN₄O₃S [M]⁺ 418.9, found 419.2. |
| 1.9 | Cl | cyclopropyl | 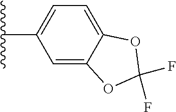 [SMILES: *c1ccc2c(c1)OC(F)(F)O2] | CH₃ | CH₃CH₂ | (300 MHz, DMSO-d₆) δ 8.12 (d, J = 5.1 Hz, 2H), 7.71 (d, J = 1.7 Hz, 1H), 7.58 (d, J = 8.3 Hz, 1H), 7.38 (dd, J = 8.1, 1.7 Hz, 1H), 7.12 (s, 1H), 4.01 (q, J = 7.4 Hz, 2H), 3.14- 3.05 (m, 1H), 2.59 (s, 3H), 1.22 (m, 5H), 1.06 (s, 2H). LC- MS: mass calculated for C₂₄H₁₉ClF₂N₄O₄S [M]⁺ 548.9, found 549.3. |
| 1.10 | Cl | cyclopropyl | 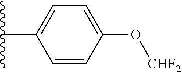 [SMILES: *c1ccc(OC(F)F)cc1] | CH₃ | CH₃CH₂ | (300 MHz, DMSO-d₆) δ 8.12 (d, J = 8.3 Hz, 2H), 7.62 (d, J = 8.4 Hz, 2H), 7.35 (d, J = 8.7 Hz, 3H), 7.12 (s, 1H), 4.01 (q, J = 7.4 Hz, 2H), 3.14-3.05 (m, 1H), 2.59 (s, 3H), 1.22 (t, J = 7.0 Hz, 5H), 1.07 (d, J = 8.1 Hz, 2H). LC-MS: mass calculated for C₂₄H₂₁ClF₂N₄O₄S [M]⁺ 534.9, found 535.2. |
| 1.11 | Cl | cyclopropyl | 4-fluorophenyl | H | isopropyl | (300 MHz, DMSO-d6) δ 9.25 (d, J = 2.2 Hz, 1H), 8.51 (d, |
| | | | | | | J = 2.2 Hz, 1H), 8.15 (s, 1H), 7.93 (dd, J = 8.6, 5.3 Hz, |
| | | | | | | 2H), 7.42 (t, J = 8.8 Hz, 2H), 7.11 (s, 1H), 4.93 (p, J = 6.8 |
| | | | | | | Hz, 1H), 3.10 (d, J = 3.7 Hz, 1H), 1.26 (dd, J = 21.9, 7.0 |
| | | | | | | Hz, 8H), 1.07 (d, J = 3.7 Hz, 2H), LC-MS: mass calculated |
| | | | | | | for C₂₃H₂₀ClFN₄O₃S [M]⁺ 486.9, found 487.2. |
| 1.12 | Cl | cyclopropyl | Br | H | CH₃CH₂ | (500 MHz) δ 9.08 (d, J = 2.3 Hz, 1H), 8.51 (d, J = 2.2 Hz, |
| | | | | | | 1H), 8.21-8.03 (m, 1H), 7.17 (dd, J = 70.7, 0.7 Hz, 1H), |
| | | | | | | 4.07 (q, J = 7.4 Hz, 2H), 3.08 (tq, J = 7.2, 4.1 Hz, 1H), |
| | | | | | | 1.32-1.12 (m, 5H), 1.12-0.95 (m, 2H). LC-MS: mass |
| | | | | | | calculated for C₁₆H₁₄BrClFN₄O₃S [M]⁺ 457.7, found 459. |
| 1.13 | Cl | cyclopropyl | 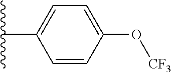 [SMILES: *c1ccc(OC(F)(F)F)cc1] | H | CH₃CH₂ | (500 MHz, DMSO-d6) o 9.27 (d, J = 2.3 Hz, 1H), 8.58 (d, J = 2.3 Hz, 1H), 8.17 (d, J = 0.6 Hz, 1H), 8.11-7.90 (m, 2H), 7.58 (d, J = 8.3 Hz, 2H), 7.33-7.06 (m, 1H), 4.08 (q, J = 7.4 Hz, 2H), 3.09 (tt, J = 7.3, 3.9 Hz, 1H), 1.24 (m, 6H), 1.12-0.97 (m, 1H). LC-MS: mass calculated for |
| | | | | | | C₂₃H₁₈ClF₃N₄O₄S [M]⁺ 538.9, found 539. |
| 1.14 | Cl | cyclopropyl | 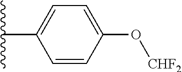 [SMILES: *c1ccc(OC(F)F)cc1] | H | CH₃CH₂ | (500 MHz, DMSO-d6) δ 9.25 (d, J = 2.3 Hz, 1H), 8.55 (d, J = 2.2 Hz, 1H), 8.15 (d, J = 0.7 Hz, 1H), 7.98-7.92 (m, 2H), 7.41-7.35 (m, 3H), 7.12 (d, J = 0.7 Hz, 1H), 4.08 (q, J = 7.4 Hz, 2H), 3.09 (td, J = 7.1, 3.6 Hz, 1H), 1.24 (dt, J = 17.0, 7.2 Hz, 6H), 1.10-1.03 (m, 2H). LC-MS: mass |
| | | | | | | calculated for C₂₃H₁₉ClF₂N₄O₄S [M]⁺ 520.9, found 521. |
| 1.15 | Cl | cyclopropyl | 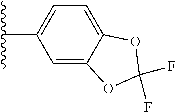 [SMILES: *c1ccc2c(c1)OC(F)(F)O2] | H | CH₃CH₂ | δ 9.24 (d, J = 2.3 Hz, 1H), 8.56 (d, J = 2.3 Hz, 1H), 8.16 (d, J = 2.5 Hz, 1H), 8.03 (d, J = 1.9 Hz, 1H), 7.73 (dd, J = 8.4, 1.8 Hz, 1H), 7.62 (d, J = 8.4 Hz, 1H), 7.12 (s, 1H), 4.05 (dq, J = 18.4, 7.3 Hz, 2H), 3.09 (dt, J = 6.9, 3.3 Hz, 1H), 1.27 (t, J = 7.4 Hz, 3H), 1.26-1.14 (m, 2H), 1.06 (s, 2H). LC-MS: mass calculated for C₂₃H₁₇ClF₂N₄O₅S [M]⁺ 534.9, found 535. |
| 1.16 | Br | cyclopropyl | 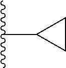 [SMILES: *C1CC1] | H | CH₃CH₂ | δ 8.69 (d, J = 2.2 Hz, 1H), 8.06-7.98 (m, 2H), 7.22 (s, 1H), 3.97 (q, J = 7.4 Hz, 2H), 3.09 (dq, J = 7.1, 3.8 Hz, 1H), 2.26-2.11 (m, 1H), 1.29-1.09 (m, 7H), 1.13-0.98 (m, 2H), 0.96-0.85 (m, 2H). LC-MS: mass calculated for C₁₉H₁₉ClFN₄O₃S [M]⁺ 418.9, found 419.2. |
| 1.17 | Cl | cyclopropyl | 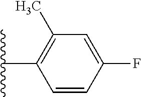 [SMILES: *c1ccc(F)cc1C] | H | CH₃CH₂ | 1H NMR (300 MHz, DMSO-d6) δ 8.95 (d, J = 2.1 Hz, 1H), 8.29 (d, J = 2.1 Hz, 1H), 8.16 (d, J = 5.7 Hz, 1H), 7.47 (dd, J = 8.5, 6.0 Hz, 1H), 7.40-7.17 (m, 2H), 7.12 (d, J = 4.0 Hz, 1H), 4.07 (p, J = 6.5, 5.8 Hz, 2H), 3.09 (m, 1H), 1.48- 1.11 (m, 6H), 1.07 (m, 2H), 0.92-0.67 (m, 2H). LC-MS: mass calculated for C₂₃H₂₀ClFN₄O₃S [M]⁺ 486.9, found 487.1. |
| 1.18 | Cl | cyclopropyl | 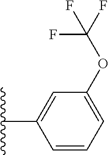 [SMILES: *c1cccc(OC(F)(F)F)c1] | H | CH₃CH₂ | 1H NMR (300 MHz, DMSO-d6) δ 9.30 (d, J = 2.3 Hz, 1H), 8.56 (dd, J = 24.3, 2.2 Hz, 1H), 8.16 (d, J = 7.6 Hz, 1H), 7.93 (d, J = 5.8 Hz, 2H), 7.72 (t, J = 8.2 Hz, 1H), 7.53 (d, J = 8.3 Hz, 1H), 7.12 (d, J = 4.7 Hz, 1H), 4.09 (q, J = 7.3 Hz, 2H), 3.20-2.99 (m, 1H), 1.39-1.11 (m, 5H), 1.06 (m, 2H). LC-MS: mass calculated for C₂₃H₁₈ClF₃N₄O₄S [M]⁺ 538.9, found 539.1. |
| 1.19 | Cl | cyclopropyl | 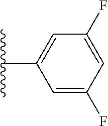 [SMILES: *c1cc(F)cc(F)c1] | H | CH₃CH₂ | 1H NMR (300 MHz, DMSO-d6) o 9.30 (d, J = 2.2 Hz, 1H), 8.61 (d, J = 2.3 Hz, 1H), 8.41 (s, 1H), 8.17 (s, 1H), 7.72 (d, J = 7.6 Hz, 1H), 7.40 (d, J = 8.4 Hz, 1H), 7.19 (d, J = 39.0 Hz, 1H), 4.08 (q, J = 7.3 Hz, 2H), 3.10 (m, 1H), 1.24 (m, 7H). LC-MS: mass calculated for C₂₂H₁₂ClF₂N₄O₃S [M]⁺ 490.9, found 491.1. |
| 1.20 | Cl | cyclopropyl | 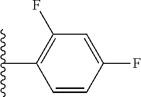 [SMILES: *c1ccc(F)cc1F] | H | CH₃CH₂ | 1H NMR (500 MHz, DMSO-d6) δ 9.21-9.00 (m, 1H), 8.60- 8.41 (m, 1H), 8.18 (s, 1H), 7.87 (td, J = 8.9, 6.4 Hz, 1H), 7.52 (ddd, J = 11.6, 9.3, 2.6 Hz, 1H), 7.33 (td, J = 8.4, 2.5 Hz, 1H), 7.12 (s, 1H), 4.20-3.94 (m, 2H), 3.09 (tt, J = 7.1, 4.0 Hz, 1H), 1.34-1.12 (m, 5H), 1.13-0.97 (m, 2H). LC- MS: mass calculated for C₂₂H₁₇ClF₂N₄O₃S [M]⁺ 490.9, found 491.1. |
| 1.21 | Cl | cyclopropyl | 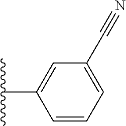 [SMILES: *c1cccc(C#N)c1] | H | CH₃CH₂ | 1H NMR (300 MHz, DMSO-d6) o 9.32 (d, J = 2.2 Hz, 1H), 8.65 (d, J = 2.2 Hz, 1H), 8.45 (s, 1H), 8.31-8.10 (m, 2H), 7.98 (d, J = 7.7Hz, 1H), 7.78 (t, J = 7.8 Hz, 1H), 7.13 (s, 1H), 4.07 (p, J = 8.3, 7.8 Hz, 2H), 3.09 (q, J = 3.2 Hz, 1H), 1.24 (dt, J = 18.8, 6.7 Hz, 5H), 1.07 (d, J = 4.2 Hz, 2H). LC-MS: mass calculated for C₂₃H₁₈ClN₅O₃S [M]⁺ 479.9, found 480. |
| 1-22 | Cl | cyclopropyl | 4-fluorophenyl | Cl | CH₃CH₂ | 1H NMR (300 MHz, DMSO-d6) 0 8.29 (s, 1H), 8.20 (s, |
| | | | | | | 1H), 7.70 (t, J = 6.9 Hz, 2H), 7.41 (t, J = 8.8 Hz, 2H), 7.14 |
| | | | | | | (s, 1H), 4.05 (q, J = 7.5 Hz, 2H), 3.08 (m, 1H), 1.25 (m, |
| | | | | | | 5H), 1.06 (m, 2H). LC-MS: mass calculated for |
| | | | | | | C₂₂H₁₂Cl₂FN₄O₃S [M]⁺ 506, found 507 [M + 1]. |
| 1-23 | Cl | cyclopropyl | 4-fluorophenyl | Cl | isopropyl | 1H NMR (500 MHz, DMSO-d6) δ 8.24 (s, 1H), 8.19 (s, |
| | | | | | | 1H), 8.15 (s, 1H), 7.72-7.63 (m, 2H), 7.44-7.36 (m, 2H), |
| | | | | | | 4.90-4.82 (m, 1H), 3.09 (tt, J = 7.2, 3.9 Hz, 1H), 1.28 (m, |
| | | | | | | 6H), 1.22 (dt, J = 7.7, 5.8 Hz, 2H), 1.09-1.03 (m, 2H). LC- |
| | | | | | | MS: mass calculated for C₂₃H₁₉Cl₂FN₄O₃S [M]⁺ 520, found |
| | | | | | | 521 [M + 1]. |
| 1-24 | Cl | cyclopropyl | 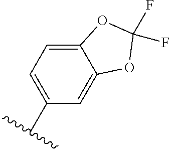 [SMILES: *c1ccc2c(c1)OC(F)(F)O2] | Cl | isopropyl | 1H NMR (500 MHz, DMSO-d6) δ 8.22-8.16 (m, 1H), 7.75 (dd, J = 12.6, 1.8 Hz, 1H), 7.60 (dd, J = 8.3, 2.7 Hz, 1H), 7.45 (ddd, J = 15.8, 8.3, 1.8 Hz, 1H), 7.12 (d, J = 0.7 Hz, 1H), 5.76 (s, 1H), 4.86 (dp, J = 9.0, 6.8 Hz, 1H), 3.09 (tt, J = 7.2, 3.9 Hz, 1H), 1.28 (dd, J = 6.9, 3.2 Hz, 6H), 1.26- 1.17 (m, 2H), 1.13-1.02 (m, 2H). LC-MS: mass calculated for C₂₄H₁₈Cl₂F₂N₄O₅S [M]⁺ 582, found 583 [M + 1]. |
| 1-25 | Cl | cyclopropyl | 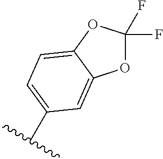 [SMILES: *c1ccc2c(c1)OC(F)(F)O2] | Me | isopropyl | 1H NMR (300 MHz, DMSO-d6) δ 8.09 (d, J = 13.8 Hz, 2H), 7.70 (d, J = 1.7 Hz, 1H), 7.57 (d, J = 8.3 Hz, 1H), 7.37 (dd, J = 8.3, 1.7 Hz, 1H), 7.10 (s, 1H), 4.84 (p, J = 6.8 Hz, 1H), 3.08 (td, J = 7.0, 3.7 Hz, 1H), 2.58 (s, 3H), 1.33-1.11 (m, 8H), 1.06 (m, 2H). LC-MS: mass calculated for C₂₅H₂₁ClF₂NO₅S [M]⁺ 562 found 563 [M + 1]. |
| 1-26 | Cl | cyclopropyl | 4-fluorophenyl | Me | isopropyl | 1H NMR (300 MHz, DMSO-d6) δ 8.12 (s, 1H), 8.06 (s, |
| | | | | | | 1H), 7.65-7.54 (m, 2H), 7.38 (td, J = 9.1, 2.6 Hz, 2H), |
| | | | | | | 7.10 (s, 1H), 4.85 (p, J = 6.9 Hz, 1H), 3.08 (td, J = 6.9, 3.5 |
| | | | | | | Hz, 1H), 2.57 (s, 3H), 1.23 (dd, J = 11.0, 6.7 Hz, 8H), 1.06 |
| | | | | | | (m, 2H). LC-MS: mass calculated for C₂₄H₂₂ClFN₄O₃S |
| | | | | | | [M]⁺ 500, found 501 [M + 1]. |
| 1-27 | Cl | cyclopropyl | 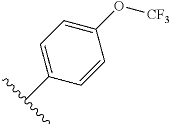 [SMILES: *c1ccc(OC(F)(F)F)cc1] | Me | CH₃CH₂ | 1H NMR (300 MHz, DMSO-d6) δ 8.13 (d, J = 3.2 Hz, 2H), 7.70 (d, J = 8.6 Hz, 2H), 7.55 (d, J = 8.2 Hz, 2H), 7.12 (s, 1H), 4.02 (q, J = 7.4 Hz, 2H), 3.09 (t, J = 5.2 Hz, 1H), 2.59 (s, 3H), 1.23 (t, J = 7.3 Hz, 5H), 1.14-1.00 (m, 2H). LC- MS: mass calculated for C₂₄H₂₀ClF₃N₄O₄S [M]⁺ 552.9, found 553.2. |
| 1-28 | Cl | cyclopropyl | phenyl | H | CH₃CH₂ | 1H NMR (300 MHz, DMSO-d6) δ 8.61-8.31 (m, 2H), 8.06- |
| | | | | | | 7.73 (m, 2H), 7.40-7.17 (m, 2H), 7.02 (d, J = 6.7 Hz, |
| | | | | | | 3H), 6.93-6.74 (m, 1H), 3.02 (q, J = 7.3 Hz, 2H), 1.26 (t, |
| | | | | | | J = 7.3 Hz, 5H), 0.93-0.59 (m, 2H). LC-MS: mass |
| | | | | | | calculated for C₂₂H₁₉ClN₄O₃S [M]⁺ 454.9, found 455. |
| 1-29 | Cl | cyclopropyl | 4-chlorophenyl | H | CH₃CH₂ | 1H NMR (500 MHz, DMSO-d6) δ 9.26 (d, J = 2.2 Hz, 1H), |
| | | | | | | 8.56 (d, J = 2.3 Hz, 1H), 8.16 (s, 1H), 7.99-7.80 (m, 2H), |
| | | | | | | 7.70-7.57 (m, 2H), 7.12 (s, 1H), 5.76 (s, 1H), 4.08 (q, J = |
| | | | | | | 7.4 Hz, 2H), 3.08 (tt, J = 7.1, 3.9 Hz, 1H), 1.25 (dt, J = |
| | | | | | | 10.8, 7.0 Hz, 3H), 1.22-1.13 (m, 2H), 1.09-0.98 (m, |
| | | | | | | 2H). LC-MS: mass calculated for C₂₂H₁₈Cₗ₂N₄O₃S [M]⁺ |
| | | | | | | 489.3, found 489.1. |
| 1-30 | Cl | cyclopropyl | 2,6- | H | CH₃CH₂ | 1H NMR (300 MHz, DMSO-d6) δ 8.95 (d, J = 2.1 Hz, 1H), |
| | | | dichlorophenyl | | | 8.34 (d, J = 2.1 Hz, 1H), 8.23 (s, 1H), 7.71 (d, J = 7.9 Hz, |
| | | | | | | 2H), 7.57 (dd, J = 8.9, 7.3 Hz, 1H), 7.13 (s, 1H), 4.09 (q, |
| | | | | | | J = 7.3 Hz, 2H), 3.17-3.00 (m, 1H), 1.21 (t, J = 6.8 Hz, |
| | | | | | | 5H), 1.07 (d, J = 4.3 Hz, 2H). LC-MS: mass calculated for |
| | | | | | | C₂₂H₁₇ClN₄O₃S [M]⁺ 523.8, found 523.2. |
| 1-31 | Cl | cyclopropyl | 2,6-difluorophenyl | H | CH₃CH₂ | 1H NMR (300 MHz, DMSO-d6) δ 9.06 (d, J = 1.9 Hz, 1H), |
| | | | | | | 8.50 (d, J = 1.8 Hz, 1H), 8.21 (s, 1H), 7.63 (p, J = 7.0 Hz, |
| | | | | | | 1H), 7.36 (t, J = 8.2 Hz, 2H), 7.13 (s, 1H), 4.09 (q, J = 7.4 |
| | | | | | | Hz, 2H), 3.08 (dd, J = 7.3, 3.7 Hz, 1H), 1.23 (t, J = 7.5 Hz, |
| | | | | | | 5H), 1.10-0.99 (m, 2H). LC-MS: mass calculated for |
| | | | | | | C₂₂H₁₇ClF₂N₄O₃S [M]⁺ 490.9, found 491.2. |
| 1-32 | Cl | cyclopropyl | 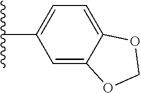 [SMILES: *c1ccc2c(c1)OCO2] | H | CH₃CH₂ | 1H NMR (300 MHz, DMSO-d6) δ 9.19 (d, J = 2.2 Hz, 1H), 8.48 (d, J = 2.2 Hz, 1H), 8.12 (s, 1H), 7.51 (d, J = 1.9 Hz, 1H), 7.36 (d, J = 7.0 Hz, 1H), 7.11 (t, J = 4.1 Hz, 1H), 6.13 (s, 2H), 4.06 (q, J = 7.3 Hz, 2H), 3.12-3.04 (m, 1H), 1.24 (q, J = 7.8 Hz, 5H), 1.05 (s, 2H). LC-MS: mass calculated for C₂₃H₁₉ClN₄O₅S [M]⁺ 498.9, found 499. |
| 1-33 | Cl | Me | 4-fluorophenyl | H | CH₃CH₂ | 1H NMR (300 MHz, DMSO-d6) δ 9.25 (d, J = 2.2 Hz, 1H), |
| | | | | | | 8.55 (d, J = 2.2 Hz, 1H), 8.18 (s, 1H), 7.94 (dd, J = 8.6, 5.4 |
| | | | | | | Hz, 2H), 7.42 (t, J = 8.8 Hz, 2H), 7.18 (s, 1H), 4.07 (q, J = |
| | | | | | | 7.4 Hz, 2H), 3.20 (s, 3H), 1.34-1.18 (m, 3H). LC-MS: |
| | | | | | | mass calculated for C₂₂H₁₇ClF₂N₄O₃S [M]⁺ 446.8, found |
| | | | | | | 447. |
| 1-34 | Cl | cyclopropyl | 4-fluorophenyl | 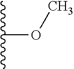 [SMILES: *OC] | CH₃CH₂ | 1H NMR (300 MHz, DMSO): δ 8.26 (s, 1H), 8.17 (s, 1H), 7.76-7.65 (m, 2H), 7.34 (t, J = 8.9 Hz, 2H), 7.11 (s, 1H), 4.03 (q, J = 6.9 Hz, 2H), 3.34 (s, 3H), 3.07 (m, 1H), 1.24 (t, J = 5.2 Hz, 5H), 1.15-0.98 (m, 2H). LC-MS: mass calculated for C₂₃H₂₀ClFN₄O₄S [M]⁺ 502, found 503 [M + 1]⁺. |
| 1-35 | Cl | cyclopropyl | 4-fluorophenyl | 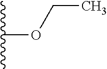 [SMILES: *OCC] | CH₃CH₂ | 1H NMR (300 MHz, CDCl3: δ 8.25 (s, 1H), 8.15 (s, 1H), 7.72-7.46 (m, 2H), 7.08 (t, J = 8.7 Hz, 2H), 6.75 (s, 1H), 4.50 (q, J = 7.0 Hz, 2H), 3.89 (q, J = 7.4 Hz, 2H), 2.98 (m, 1H), 1.32 (m, 8H), 1.00 (m, 2H). LC-MS: mass calculated for C₂₄H₂₂ClFN₄O₄S [M]⁺ 516, found 517 [M + 1]⁺. |
| 1-36 | Cl | cyclopropyl | 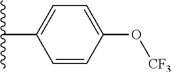 [SMILES: *c1ccc(OC(F)(F)F)cc1] | H | Isopropyl | 1H NMR (300 MHz, DMSO-d6) δ 9.27 (d, J = 2.2 Hz, 1H), 8.54 (d, J = 2.2 Hz, 1H), 8.16 (s, 1H), 8.12-7.96 (m, 2H), 7.57 (d, J = 8.3 Hz, 2H), 7.11 (s, 1H), 4.94 (p, J = 6.9 Hz, 1H), 3.13-3.02 (m, 1H), 1.33-1.22 (m, 6H), 1.19 (m, 2H), 1.05 (m, 2H). LC-MS: mass calculated for |
| | | | | | | C₂₄H₂₀ClF₃N₄O₄S [M]⁺ 552, found 553 [M + 1]⁺. |
| 1-37 | Cl | cyclopropyl | 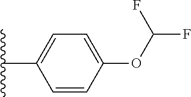 [SMILES: *c1ccc(OC(F)F)cc1] | H | Isopropyl | 1H NMR (300 MHz, DMSO-d6) δ 9.25 (d, J = 2.2 Hz, 1H), 8.51 (d, J = 2.2 Hz, 1H), 8.15 (s, 1H), 8.00-7.88 (m, 2H), 7.61 (s, 1H) 7.42-7.33 (m, 2H), 7.11 (d, J = 4.3 Hz, 1H), 4.93 (p, J = 6.8 Hz, 1H), 3.08 (td, J = 7.2, 3.8 Hz, 1H), 1.33-1.20 (m, 6H), 1.20-1.12 (m, 2H), 1.07 (m, 2H). LC-MS: mass calculated for C₂₄H₂₁ClF₂N₄O₄S [M]⁺ 534, found 535 [M + 1]⁺. |
| 1-38 | Cl | cyclopropyl | 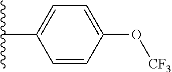 [SMILES: *c1ccc(OC(F)(F)F)cc1] | Me | Isopropyl | 1H NMR (500 MHz, DMSO-d6) δ 8.13 (s, 1H), 8.10 (s, 1H), 7.73-7.66 (m, 2H), 7.60-7.52 (m, 2H), 7.11 (d, J = 0.6 Hz, 1H), 4.86 (p, J = 6.9 Hz, 1H), 3.09 (tt, J = 7.1, 3.9 Hz, 1H), 2.59 (s, 3H), 1.32-1.18 (m, 8H), 1.13-1.03 (m, 2H). LC-MS: mass calculated for C₂₅H₂₂ClF₃N₄O₄S [M]⁺ 566, found 567 [M + 1]⁺. |
| 1-39 | Cl | cyclopropyl | 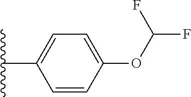 [SMILES: *c1ccc(OC(F)F)cc1] | Me | Isopropyl | 1H NMR (300 MHz, DMSO-d6) δ 8.09 (d, J = 17.1 Hz, 2H), 7.66-7.57 (m, 2H), 7.35 (d, J = 9.1 Hz, 3H), 7.11 (d, J = 2.8 Hz, 1H), 4.85 (p, J = 6.8 Hz, 1H), 3.08 (dq, J = 7.1, 4.0, 3.5 Hz, 1H), 2.59 (s, 3H), 1.24 (t, J = 8.6 Hz, 8H), 1.06 (d, J = 4.3 Hz, 2H). LC-MS: mass calculated for C₂₅H₂₃ClF₂N₄O₄S [M]⁺ 548, found 549 [M + 1]⁺. |
| 1-40 | Cl | 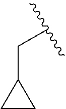 [SMILES: *CC1CC1] | 4-fluorophenyl | H | CH₃CH₂ | 1H NMR (300 MHz, CDCl3) δ 9.07 (d, J = 2.2 Hz, 1H), 8.67 (d, J = 2.2 Hz, 1H), 8.37 (s, 1H), 7.69 (dd, J = 8.6, 5.2 Hz, 2H), 7.24 (d, J = 8.5 Hz, 2H), 6.83 (s, 1H), 4.22 (d, J = 7.2 Hz, 2H), 3.94 (q, J = 7.4 Hz, 2H), 1.38 (t, J = 7.4 Hz, 4H), 0.72-0.44 (m, 4H). LC-MS: mass calculated for C₂₃H₂₀ClFN₄O₃S [M]⁺ 486, found 487 [M + 1]⁺. |
| 1-41 | Cl | cyclopropyl | 4-chlorophenyl | Me | Isopropyl | 1H NMR (300 MHz, DMSO-d6) δ 8.10 (d, J = 16.6 Hz, |
| | | | | | | 2H), 7.68-7.53 (m, 4H), 7.11 (s, 1H), 4.85 (p, J = 6.8 Hz, |
| | | | | | | 1H), 3.14-3.05 (m, 1H), 2.58 (s, 3H), 1.33-1.18 (m, 8H), |
| | | | | | | 1.18-1.00 (m, 2H). LC-MS: mass calculated for |
| | | | | | | C₂₄H₂₂Cl₂N₄O₃S [M]₊ 516, found 517 [M + 1]⁺. |
| 1-42 | Cl | 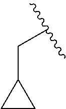 [SMILES: *CC1CC1] | 4-fluorophenyl | Me | CH₃CH₂ | 1H NMR (500 MHz, CDCl3) δ 8.22 (d, J = 4.5 Hz, 2H), 7.36-7.27 (m, 2H), 7.17-7.07 (m, 2H), 6.72 (s, 1H), 4.13 (d, J = 7.3 Hz, 2H), 3.81 (q, J = 7.4 Hz, 2H), 2.56 (s, 3H), 1.27 (t, J = 7.4 Hz, 4H), 0.68-0.40 (m, 4H). LC-MS: mass calculated for C₂₄H₂₂ClFN₄O₃S [M]⁺ 516, found 517 [M + 1]⁺. |
| I-43 | Cl | cyclopropyl | 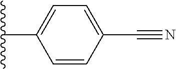 [SMILES: *c1ccc(C#N)cc1] | H | CH₃CH₂ | 1H NMR (300 MHz, DMSO-d6) δ 9.25 (d, J = 2.2 Hz, 1H), 8.56 (d, J = 2.2 Hz, 1H), 8.11 (s, 1H), 8.01 (q, J = 8.4 Hz, 4H), 7.06 (s, 1H), 4.03 (q, J = 7.4 Hz, 2H), 3.01 (td, J = 6.9, 3.5 Hz, 1H), 1.30-1.14 (m, 5H), 1.14-0.92 (m, 2H). LC-MS: mass calculated for C₂₃H₁₈ClN₅O₃S [M]⁺ 478, found 479 [M + 1]⁺. |
| 1-44 | Cl | cyclopropyl | 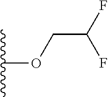 [SMILES: *OCC(F)F] | H | CH₃CH₂ | 1H NMR (300 MHz, Chloroform-d) δ 8.62 (d, J = 2.9 Hz, 1H), 8.21 (s, 1H), 8.02 (d, J = 2.9 Hz, 1H), 6.77 (s, 1H), 6.18 (tt, J = 3.9 Hz, 1H), 4.39 (td, J = 12.8, 3.9 Hz, 2H), 3.86 (q, J = 7.4 Hz, 2H), 3.06 (tt, J = 7.2, 3.8 Hz, 1H), 1.35 (h, J = 8.7, 8.1 Hz, 5H), 1.15-1.03 (m, 2H). LC-MS: mass calculated for C₁₈H₁₇ClF₂N₄O₃S [M]⁺ 458, found 459 [M + 1]⁺. |

The wavy line denotes the bond to the remainder of the molecule

Analogously, with appropriate modification of the starting materials or intermediates thereof, the procedures as described in the preparation example above were used to obtain further compounds of formulae I.A.2 listed in Table C with their physical data.

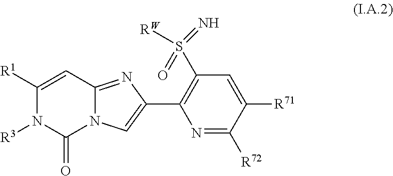

TABLE C

| No | R¹ | R³ | R⁷¹ | R⁷² | R^{W} | ¹H-NMR data (δ); LCMS [RT (min), M⁺] |
|---|---|---|---|---|---|---|
| I-45 | Cl | cyclopropyl | —Br | H | CH₃CH₂ | 1H NMR (300 MHz, DMSO-d6) δ 8.99 (d, J = 2.2 Hz, 1H), |
| | | | | | | 8.54 (d, J = 2.2 Hz, 1H), 8.11 (s, 1H), 7.10 (s, 1H), 4.56 (s, |
| | | | | | | 1H), 3.75 (qd, J = 7.2, 6.5, 3.3 Hz, 2H), 3.21-3.00 (m, |
| | | | | | | 1H), 1.17 (m, 5H), 1.04 (m, 2H). LC-MS: mass calculated |
| | | | | | | for C₁₆H₁₅BrClN₅O₂S [M]⁺ 455, found 456 [M + 1]⁺ |
| I-46 | Cl | cyclopropyl | 4-fluorophenyl | H | CH₃CH₂ | 1H NMR (300 MHz, DMSO-d6) δ 9.14 (d, J = 2.1 Hz, 1H), |
| | | | | | | 8.59 (d, J = 2.2 Hz, 1H), 8.12 (s, 1H), 7.98-7.87 (m, 2H), |
| | | | | | | 7.42 (t, J = 8.8 Hz, 2H), 7.12 (s, 1H), 4.53 (s, 1H), 3.83 (dd, |
| | | | | | | J = 13.9, 7.4 Hz, 1H), 3.73 (dd, J = 13.9, 7.2 Hz, 1H), 3.08 |
| | | | | | | (tt, J = 7.2, 4.0 Hz, 1H), 1.19 (m, 5H), 1.05 (p, J = 5.6, 5.0 |
| | | | | | | Hz, 2H). LC-MS: mass calculated for C₂₂H₁₉ClFN₅O₂S [M]⁺ |
| | | | | | | 471, found 472 [M + 1]⁺. |

Analogously, with appropriate modification of the starting materials or intermediates thereof, the procedures as described in the preparation example above were used to obtain further compounds of formulae I.A.3 listed in Table D with their physical data.

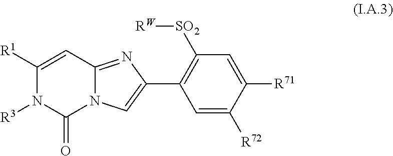

TABLE D

| No. | R¹ | R³ | R⁷¹ | R⁷² | R^{W} | ¹H-NMR data (δ); LCMS [RT (min), M⁺] |
|---|---|---|---|---|---|---|
| 1-47 | Cl | cyclopropyl | 4-fluorophenyl | H | CH₃CH₂ | 1H NMR (300 MHz, DMSO-d6) δ 8.24 (d, J = 2.0 Hz, 1H), |
| | | | | | | 8.16 (s, 1H), 8.09 (dd, J = 8.1, 2.0 Hz, 1H), 7.94-7.78 (m, |
| | | | | | | 3H), 7.45-7.31 (m, 2H), 7.09 (s, 1H), 3.66 (q, J = 7.3 Hz, |
| | | | | | | 2H), 3.07 (tt, J = 7.2, 4.0 Hz, 1H), 1.28-0.98 (m, 7H). LC- |
| | | | | | | MS: mass calculated for C₂₃H₁₉ClFN₃O₃S [M]⁺ 471, found |
| | | | | | | 472 [M + 1]⁺. |
| 1-48 | Cl | cyclopropyl | 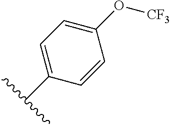 [SMILES: *c1ccc(OC(F)(F)F)cc1] | H | CH₃CH₂ | 1H NMR (300 MHz, DMSO-d6) δ 8.27 (d, J = 2.0 Hz, 1H), 8.21-8.08 (m, 2H), 7.91 (m, 3H), 7.54 (d, J = 8.3 Hz, 2H), 7.10 (s, 1H), 3.68 (q, 7.4 Hz, 2H), 3.07 (dq, J = 7.1, 3.9 Hz, 1H), 1.28-0.99 (m, 7H). LC-MS: mass calculated for C₂₄H₁₉ClF₃N₃O₄S [M]⁺ 537, found 538 [M + 1]⁺. |
| 1-49 | Cl | cyclopropyl | 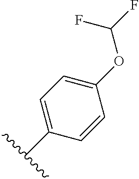 [SMILES: *c1ccc(OC(F)F)cc1] | H | CH₃CH₂ | 1H NMR (300 MHz, DMSO-d6) δ 8.25 (d, J = 2.0 Hz, 1H), 8.21-8.02 (m, 2H), 7.96-7.60 (m, 3H), 7.49-7.21 (m, 3H), 7.10 (d, J = 1.8 Hz, 1H), 3.66 (q, J = 7.3 Hz, 2H), 3.08 (tt, J = 7.0, 3.9 Hz, 1H), 1.41-1.00 (m, 7H). LC-MS: mass calculated for C₂₄H₂₀ClF₂N₃O₄S [M]⁺ 519, found 520 [M + 1]⁺. |
| 1-50 | Cl | cyclopropyl | 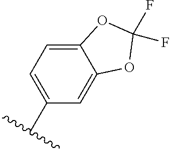 [SMILES: *c1ccc2c(c1)OC(F)(F)O2] | H | CH₃CH₂ | 1H NMR (300 MHz, DMSO-d6) δ 8.23 (d, J = 2.0 Hz, 1H), 8.15-8.03 (m, 2H), 7.92-7.80 (m, 2H), 7.61 (dd, J = 8.6, 1.8 Hz, 1H), 7.58-7.49 (m, 1H), 7.04 (d, J = 1.9 Hz, 1H), 3.61 (q, J = 7.1 Hz, 2H), 3.09 (dt, J = 6.9, 3.3 Hz, 1H), 1.24 (t, J = 6.9 Hz, 2H), 1.14 (t, J = 7.3 Hz, 3H), 1.04 (m, 2H). LC-MS: mass calculated for C₂₄H₁₈ClF₂N₂O₅S [M]⁺ 533, found 534 [M+1]⁺. |

### B. BIOLOGICAL EXAMPLES

The activity of the compounds of formula (I) of the invention could be demonstrated and evaluated in biological tests described in the following. If not otherwise specified, the test solutions are prepared as follows: The active compound is dissolved at the desired concentration in a mixture of 1:1 (vol:vol) distilled water:acteone. The test solution is prepared at the day of use. Test solutions are prepared in general at concentrations of 2500 ppm, 800 ppm, and 300 ppm (wt/vol).

B.1 Boll Weevil (Anthonomus grandis)

For evaluating control of boll weevil (Anthonomus grandis) the test unit consisted of 96-well-microtiter plates containing an insect diet and 5-10 A. grandis eggs. The compounds were formulated using a solution containing 75% v/v water and 25% v/v DMSO. Different concentrations of formulated compounds were sprayed onto the insect diet at 5 μl, using a custom-built micro atomizer, at two replications. After application, microtiter plates were incubated at about 25±1° C. and about 75±5% relative humidity for 5 days. Egg and larval mortality was then visually assessed. In this test, compounds I.1, I.2, I.3, I.4, I.5, I.6, I.8, I.9, I.10, I.11, I.12, I.13, I.14, I.15, I.16, I.17, I.18, I.19, I.20, I.21, I.22, I.25, I.26, I.27, I.28, I.29, I.32, I.33, I.46, I.47, I.48, I.49, I.50 resp., at 800 ppm showed over 75% mortality in comparison with untreated controls.

B.2 Tobacco Budworm (Heliothis virescens)

For evaluating control of tobacco budworm (Heliothis virescens) the test unit consisted of 96-well-microtiter plates containing an insect diet and 15-25 H. virescens eggs. The compounds were formulated using a solution containing 75% v/v water and 25% v/v DMSO. Different concentrations of formulated compounds were sprayed onto the insect diet at 10 μl, using a custom-built micro atomizer, at two replications. After application, microtiter plates were incubated at about 28±1° C. and about 80±5% relative humidity for 5 days. Egg and larval mortality was then visually assessed. In this test, compounds I.1, I.4, I.5, I.6, I.8, I.9, I.10, I.11, I.12, I.13, I.14, I.15, I.16, I.17, I.18, I.19, I.20, I.21, I.22, I.23, I.25, I.26, I.27, I.28, I.29, I.30, I.32, I.36, I.37, I.47, I.48, I.49, I.50 resp., at 800 ppm showed over 75% mortality in comparison with untreated controls.

B.3 Green Peach Aphid (Myzus persicae)

For evaluating control of green peach aphid (Myzus persicae) through systemic means the test unit consisted of 96-well-microtiter plates containing liquid artificial diet under an artificial membrane. The compounds were formulated using a solution containing 75% v/v water and 25% v/v DMSO. Different concentrations of formulated compounds were pipetted into the aphid diet, using a custom built pipetter, at two replications. After application, 5-8 adult aphids were placed on the artificial membrane inside the microtiter plate wells. The aphids were then allowed to suck on the treated aphid diet and incubated at about 23±1° C. and about 50±5% relative humidity for 3 days. Aphid mortality and fecundity was then visually assessed. In this test, compounds I.1, I.2, I.3, I.4, I.5, I.6, I.7, I.8, I.10, I.11, I.12, I.13, I.14, I.16, I.17, I.18, I.19, I.20, I.21, I.25, I.26, I.27, I.28, I.29, I.31, I.32, I.33, I.36, I.37, I.46, I.47, I.48, I.49, resp., at 800 ppm showed over 75% mortality in comparison with untreated controls.

B.4 Diamond Back Moth (Plutella xylostella)

The active compound is dissolved at the desired concentration in a mixture of 1:1 (vol:vol) distilled water: acetone. Surfactant (Kinetic) is added at a rate of 0.01% (vol/vol). The test solution is prepared at the day of use. Cabbage leaf discs (60 mm in diameter) are dipped in test solution and air-dried. Treated leaves are placed in petri dishes lined with moistened filter paper and inoculated with ten 3^{rd} instar larvae. Mortality is recorded 72 hours after treatment. Feeding damage is also recorded using a scale of 0-100%. In this test, compounds I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23, I.25, I.26, I.27, I.28, I.29, I.30, I.31, I.32, I.33, I.36, I.37, I.45, I.46, I.47, I.48, I.49, I.50 respectively, at 800 ppm showed over 75% mortality in comparison with untreated controls.

B.5 Southern Armyworm (Spodoptera eridania), 2^{nd} Instar Larvae

The active compounds were formulated by a Tecan liquid handler in 100% cyclohexanone as a 10,000 ppm solution supplied in tubes. The 10,000 ppm solution was serially diluted in 100% cyclohexanone to make interim solutions. These served as stock solutions for which final dilutions were made by the Tecan in 50% acetone:50% water (v/v) into 10 or 20 ml glass vials. A nonionic surfactant (Kinetic®) was included in the solution at a volume of 0.01% (v/v). The vials were then inserted into an automated electrostatic sprayer equipped with an atomizing nozzle for application to plants/insects. Lima bean plants (variety Sieva) were grown 2 plants to a pot and selected for treatment at the 1^{st} true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. Ten to 11 armyworm larvae were placed into the bag and the bags zipped closed. Test plants were maintained in a growth room at about 25° C. and about 20-40% relative humidity for 4 days, avoiding direct exposure to fluorescent light (14:10 light:dark photoperiod) to prevent trapping of heat inside the bags. Mortality and reduced feeding were assessed 4 days after treatment, compared to untreated control plants. In this test, compounds I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.25, I.26, I.27, I.28, I.29, I.30, I.31, I.32, I.34, I.35, I.36, I.37, I.38, I.39, I.40, I.41, I-44, I.45, I.46, I.47, I.48, I.49, I.50 respectively, at 300 ppm showed over 75% mortality in comparison with untreated controls.

### Comparison Data:

| | Test according to B.4 |
|---|---|
| | (Plutella xylostella)) |
| Compound | @ 10 ppm |
| Compound 1.3 of present application | 100% |
| 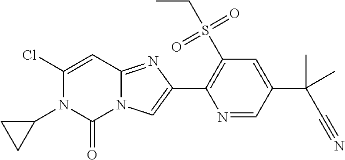 [SMILES: CCS(=O)(=O)c1cc(C(C)(C)C#N)cnc1-c1cn2c(=O)n(C3CC3)c(Cl)cc2n1] | |
| Compound C.4 of WO2021204577 | 25% |
| 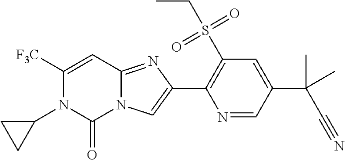 [SMILES: CCS(=O)(=O)c1cc(C(C)(C)C#N)cnc1-c1cn2c(=O)n(C3CC3)c(C(F)(F)F)cc2n1] | |

The comparison data show that the compound of the present applications have a significantly better activity, despite only small structural differences (Cl/CF₃ in this case).

1. A compound of formula (I) 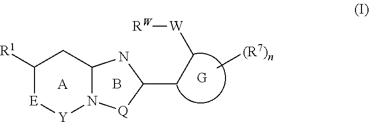
wherein
rings A and B are fully unsaturated;
Y is C═X, wherein X is O;
E is N(R³);
Q is N, N(R⁵), or CH;
G is phenyl or pyridyl;
W is S, S(O), S(O)₂, S(O)(NR^{W}), or S(O)(NH);
R¹ is halogen;
R³, R⁵ are independently C₁-C₆-alkyl, C₁-C₆-alkoxy, C₂-C₆-alkenyl, C₂-C₆-alkynyl, C₃-C₆-cycloalkyl, C₃-C₆-cycloalkyl-C₁-C₄-alkyl, which are unsubstituted or halogenated;
each R⁷ is independently H, halogen, OH, CN;
C₁-C₆-alkyl, C₃-C₆-cycloalkyl, C₂-C₆-alkenyl, C₂-C₆-alkynyl, which groups are unsubstituted or halogenated;
a 3- to 12-membered saturated, partially unsaturated, or fully unsaturated heterocyclic ring or ring system, wherein said heterocyclic ring or ring system comprises one or more, same or different heteroatoms O, N, or S, and is unsubstituted or substituted with one or more R^{H};
phenyl, which is unsubstituted or substituted with one or more R^{J};
OR^{K};
—C(CN)(CH₃)₂, or 1-cyanocyclopropyl.
each R^{H} is independently halogen, CN; C₁-C₃-alkyl, C₁-C₃-haloalkyl;
each R^{J} is independently halogen, CN; C₁-C₆-alkyl, C₁-C₆-alkoxy, which groups are unsubstituted or halogenated; or in case of two adjacent substituents R^{J} which are C₁-C₆-alkoxy, the substituents form an alkyl bridge between the two oxygens, which is unsubstituted or halogenated;
each R^{K} is C₁-C₆-alkyl, C₂-C₆-alkenyl, C₂-C₆-alkynyl, C₃-C₆-cycloalkyl, which groups are unsubstituted or substituted with one or more, same or different substituents selected from halogen and CN;
each R^{W} is independently C₁-Cᵣ-alkyl, or cyclopropyl;
index n is 1, 2, or 3, or 4, if G is phenyl, or 1, 2, or 3 if G is pyridyl;
and an N-oxide, stereoisomer, tautomer, and an agriculturally or veterinarily acceptable salt thereof.

2. The compound of formula (I) according to claim 1, wherein the compound of formula (I) is a compound of formula (I.A); 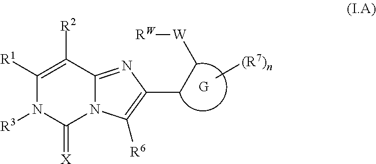
wherein all variables have a meaning as defined for formula (I).

3. The compound of formula (I.A) according to claim 2, wherein
R³ is C₁-C₃-alkyl or C₃-C₆cycloalkyl, which groups are unsubstituted or halogenated.

4. The compound of formula (I) according to claim 1, wherein G is 2-pyridyl.

5. The compound of formula (I) according to claim 1, wherein
R^{W} is C₁-C₃-alkyl.

6. The compound of formula (I) according to claim 1, wherein the index n is 1 or 2.

7. The compound of formula (I) according to claim 1, wherein R⁷ is
halogen;
C₁-C₃-alkyl, cyclopropyl;
a 6- to 9-membered saturated, partially unsaturated, or fully unsaturated heterocyclic ring or ring system, wherein said heterocyclic ring or ring system comprises one or more, same or different heteroatoms O, or N, and is unsubstituted or substituted with one or more substituents selected from halogen, CN, C₁-C₃-alkyl, and C₁-C₃-haloalkyl;
phenyl, which is unsubstituted, or substituted with one or more R^{J}; or
OR^{K}, wherein R^{K} is C₁-C₃-alkyl, which is unsubstituted or substituted with CN.

8. The compound of formula (I) according to claim 1, wherein R⁷ is selected from
—C(CN)(CH₃)₂, and 1-cyanocyclopropyl.

9. A pesticidal composition comprising the compound of formula (I), as defined in claim 1, an N-oxide, or an agriculturally acceptable salt thereof and a further pesticidal ingredient.

10. (canceled)

11. A method for combating or controlling an invertebrate pest comprising contacting said pest or its food supply, habitat, or breeding grounds with a pesticidally effective amount of at least one compound of the formula (I) according to claim 1.

12. A method for protecting growing plants from attack or infestation by an invertebrate pest comprising contacting a plant, or soil or water in which the plant is growing, with a pesticidally effective amount of at least one compound of the formula (I), according to claim 1.

13. A seed comprising a compound of the formula (I), as defined in claim 1, in an amount of from 0.1 g to 10 kg per 100 kg of seed.

14. (canceled)

15. A method for treating or protecting an animal from infestation or infection by an invertebrate pest comprising bringing the animal in contact with a pesticidally effective amount of a compound of the formula (I) as defined in claim 1.
